SEQUENCE LISTING (8 sequences)

SEQ ID NO: 1 - DNA, 555 bp, Thermoanaerobacter tengcongensis
atgaaggggacaatcgtcgggacatggataaagaccctgagggacctttacgggaatgatgtggttgatgaatctttaaaaagtgtgggttgggaaccagatagggtaattacacctctggaggatattgatgacgatgaggttaggagaatttttgctaaggtgagtgaaaaaactggtaaaaatgtcaacgaaatatggagagaggtaggaaggcagaacataaaaactttcagcgaatggtttccctcctattttgcagggagaaggctagtgaattttttaatgatgatggatgaggtacacctacagcttaccaagatgataaaaggagccactcctccaaggcttattgcaaagcctgttgcaaaagatgccattgaaatggagtacgtttctaaaagaaagatgtacgattactttttagggcttatagagggtagttctaaatttttcaaggaagaaatttcagtggaagaggtcgaaagaggcgaaaaagatggcttttcaaggctaaaagtcaggataaaatttaaaaaccccgtttttgagtga

SEQ ID NO: 2 - protein, 184 aa, Thermoanaerobacter tengcongensis
MKGTIVGTWIKTLRDLYGNDVVDESLKSVGWEPDRVITPLEDIDDDEVRRIFAKVSEKTGKNVNEIWREVGRQNIKTFSEWFPSYFAGRRLVNFLMMMDEVHLQLTKMIKGATPPRLIAKPVAKDAIEMEYVSKRKMYDYFLGLIEGSSKFFKEEISVEEVERGEKDGFSRLKVRIKFKNPVFE

SEQ ID NO: 3 - DNA, 81 bp, Thermoanaerobacter tengcongensis
ggttatattcctgaagctccaagagatgggcaagcttacgttcgtaaagatggcgaatgggtattactttctaccttttta

SEQ ID NO: 4 - protein, 28 aa, Thermoanaerobacter tengcongensis
GYIPEAPRDGQAYVRKDGEWVLLSTFPL

SEQ ID NO: 5 - DNA, 555 bp, Thermoanaerobacter tengcongensis
atgaaggggacaatcgtcgggacatggataaagaccctgagggacctttacgggaatgatgtggttgatgaatctttaaaaagtgtgggttgggaaccagatagggtaattacacctctggaggatattgatgacgatgaggttaggagaatttttgctaaggtgagtgaaaaaactggtaaaaatgtcaacgaaatatggagagaggtaggaaggcagaacataaaaactttcagcgaatggtttccctcctattttgcagggagaaggctagtgaattttttaatgatgatggatgaggtacacctacagcttaccaagatgataaaaggagccactcctccaaggcttattgcaaagcctgttgcaaaagatgccattgaaatggagtacgtttctaaaagaaagatgtacgattactttttagggtttatagagggtagttctaaatttttcaaggaagaaatttcagtggaagaggtcgaaagaggcgaaaaagatggcttttcaaggctaaaagtcaggataaaatttaaaaaccccgtttttgagtga

SEQ ID NO: 6 - protein, 184 aa, Thermoanaerobacter tengcongensis
MKGTIVGTWIKTLRDLYGNDVVDESLKSVGWEPDRVITPLEDIDDDEVRRIFAKVSEKTGKNVNEIWREVGRQNIKTFSEWFPSYFAGRRLVNFLMMMDEVHLQLTKMIKGATPPRLIAKPVAKDAIEMEYVSKRKMYDYFLGFIEGSSKFFKEEISVEEVERGEKDGFSRLKVRIKFKNPVFE

SEQ ID NO: 7 - DNA, 663 bp, Thermoanaerobacter tengcongensis
atgaaggggacaatcgtcgggacatggataaagaccctgagggacctttacgggaatgatgtggttgatgaatctttaaaaagtgtgggttgggaaccagatagggtaattacacctctggaggatattgatgacgatgaggttaggagaatttttgctaaggtgagtgaaaaaactggtaaaaatgtcaacgaaatatggagagaggtaggaaggcagaacataaaaactttcagcgaatggtttccctcctattttgcagggagaaggctagtgaattttttaatgatgatggatgaggtacacctacagcttaccaagatgataaaaggagccactcctccaaggcttattgcaaagcctgttgcaaaagatgccattgaaatggagtacgtttctaaaagaaagatgtacgattactttttagggtttatagagggtagttctaaatttttcaaggaagaaatttcagtggaagaggtcgaaagaggcgaaaaagatggcttttcaaggctaaaagtcaggataaaatttaaaaaccccgtttttgagtataagaaaaatctcgagggcagcggcggttatattcctgaagctccaagagatgggcaggcttacgttcgtaaagatggcgaatgggtattactttctacctttttatga

SEQ ID NO: 8 - protein, 221 aa, Thermoanaerobacter tengcongensis
MKGTIVGTWIKTLRDLYGNDVVDESLKSVGWEPDRVITPLEDIDDDEVRRIFAKVSEKTGKNVNEIWREVGRQNIKTFSEWFPSYFAGRRLVNFLMMMDEVHLQLTKMIKGATPPRLIAKPVAKDAIEMEYVSKRKMYDYFLGFIEGSSKFFKEEISVEEVERGEKDGFSRLKVRIKFKNPVFEYKKNLEGSGGYIPEAPRDGQAYVRKDGEWVLLSTFPL

(19) United States
(12) Patent Application Publication
(10) Pub. No.: US 2021/0260155 A1
(43) Pub. Date: Aug. 26, 2021

(54) H-NOX Proteins for Treating Cardiovascular and Pulmonary Conditions

(71) Applicant: OMNIOX.INC., San Carlos, CA (US)

(72) Inventors: Stephen P.L. Cary, Sam Mateo, CA (US); Natacha Le Moan, San Francisco, CA (US); Ana Krtolica, Los Gatos, CA (US); Jonathan A. Winger, San Francisco, CA (US)

(21) Appl. No.: 17/175,336

(22) Filed: Feb. 12, 2021

Related U.S. Application Data
(63) Continuation of application No. PCT/US2019/046519, filed on Aug. 14, 2019.
(60) Provisional application No. 62/764,797, filed on Aug. 15, 2018.
(60) Provisional application No. 62/747,547, filed on Oct. 18, 2018.

(51) Int. Cl. A61K 38/16; A61K 47/60

(52) U.S. Cl. CPC A61K 38/164; A61K 31/137; A61K 47/60

(57) ABSTRACT

Described herein are methods for treating cardiovascular and pulmonary conditions, e.g., those associated with hypoxia, or treating a subject undergoing cardiac or respiratory arrest or cardiopulmonary resuscitation, using an H-NOX protein (or a mixture of H-NOX proteins), or using a combination of an H-NOX protein (or a mixture of H-NOX proteins) and a catecholamine (such as epinephrine or norepinephrine). Also described are compositions comprising an H-NOX protein (or a mixture of H-NOX proteins) and a catecholamine (such as epinephrine or norepinephrine).

### 1. CROSS-REFERENCE TO RELATED APPLICATIONS

This application claims the benefit of U.S. Provisional Patent Application No. 62/764,797, filed Aug. 15, 2018, and U.S. Provisional Patent Application No. 62/747,547, filed Oct. 18, 2018, each of which is incorporated herein by reference in its entirety.

### 2. SEQUENCE LISTING

This application incorporates by reference a Sequence Listing submitted with this application as an ASCII text file, entitled “14521-029-228_SEQ_LISTING.txt created on Aug. 9, 2019 and having size of 8,456 bytes.

### 3. FIELD

The invention relates to treatment of cardiovascular disease and pulmonary diseases and disorders (such as those associated with hypoxia), or treatment of a subject undergoing cardiac or respiratory arrest or cardiopulmonary resuscitation, by administration of H-NOX protein (or a mixture of H-NOX proteins), preferably by administration of both an H-NOX protein (or a mixture of H-NOX proteins) and a catecholamine (such as epinephrine or norepinephrine). The invention further relates to compositions comprising an H-NOX protein or proteins (or a mixture of H-NOX proteins) and a catecholamine (such as epinephrine or norepinephrine).

### 4. BACKGROUND

H-NOX proteins (named for Heme-Nitric oxide and Oxygen binding domain) are members of a highly-conserved, well-characterized family of hemoproteins (Iyer, L. M. et al. (2003) BMC Genomics 4(1):5; Karow, D. S. et al. (2004) Biochemistry 43(31):10203-10211; Boon, E. M. et al. (2005) Nature Chem. Biol. 1:53-59; Boon, E. M. et al. (2005) Curr. Opin. Chem. Biol. 9(5):441-446; Boon, E. M. et al. (2005) J. Inorg. Biochem. 99(4):892-902, Cary, S. P. et al. (2005) Proc. Natl. Act. Sci. USA 102(37):13064-9; Karow D. S. et al. (2005) Biochemistry 44(49):16266-74; Cary, S. P. et al. (2006) Trends Biochem. Sci. 31(4):231-9; Boon, E. M. et al. (2006) J. Biol. Chem. 281(31):21892-902; Winger, J. A. et al. (2007) J. Bio. Chem. 282(2):897-907). H-NOX proteins are nitric-oxide-neutral, unlike previous hemoglobin-based oxygen carriers, H-NOX do not scavenge circulating nitric oxide, and thus are not associated with hypertensive or renal side effects. The intrinsic low NO reactivity (and high NO stability) makes wild-type and mutant H-NOX proteins desirable blood substitutes because of the lower probability of inactivation of H-NOX proteins by endogenous NO and the lower probability of scavenging of endogenous NO by H-NOX proteins. Importantly, the presence of a distal pocket tyrosine in some H-NOX proteins (Pellicena, P. et al. (2004) Proc. Natl. Acad Sci. USA 101(35):12854-12859) is suggestive of undesirable, high NO reactivity, contraindicating use as a blood substitute. For example, by analogy, a Mycobacterium tuberculosis hemoglobin protein, with a structurally analogous distal pocket tyrosine, reacts extremely rapidly with NO, and is used by the Mycobacterium to effectively scavenge and avoid defensive NO produced by an infected host (Ouellet, H. et al. (2002) Proc. Natl. Acad. Sci. USA 99(9):5902-5907). However, it was surprisingly discovered that H-NOX proteins actually have a much lower NO reactivity than that of hemoglobin making their use as blood substitutes possible.

It was discovered that H-NOX proteins that bind NO but not O₂ can be converted to H-NOX proteins that bind both NO and O₂ by the introduction of a single amino acid mutation (see International Application Publications No. WO 2007/139791 and WO 2007/139767). Thus, the affinity of H-NOX proteins for O₂ and NO and the ability of H-NOX proteins to discriminate between O₂ and NO ligands can be altered by the introduction of one or more amino acid mutations, allowing H-NOX proteins to be tailored to bind O₂ or NO with desired affinities. Additional mutations can be introduced to further alter the affinity for O₂ and/or NO. The H-NOX protein family can therefore be manipulated to exhibit improved or optimal kinetic and thermodynamic properties for O₂ delivery. For example, mutant H-NOX proteins have been generated with altered dissociation constants and/or off rates for O₂ binding that improve the usefulness of H-NOX proteins for a variety of clinical and industrial applications. H-NOX oxygen binding proteins with different “tuned” oxygen affinities has enabled the construction of a panel of H-NOX oxygen carriers with properties that are acceptable to a wide range of specific hypoxic/ischemic conditions. The ability to tune H-NOX proteins to bind and deliver O₂ is a therapeutic avenue that addresses and overcomes the central shortcomings of current O₂ carriers. Polymeric H-NOX proteins and methods to use polymeric H-NOX proteins are provided by International Application Publications WO 2014/107171 and US 20150273024.

Inadequate oxygen (O₂) delivery relative to metabolic demand leads to progressive bioenergetics collapse and cellular dysfunction. When systemic, this defines the clinical entity of shock, a major cause of morbidity and mortality in both adults and children (Kutko M. C. et al., (2003) Pediatr. Crit. Care Med. 4:333-337; Martin G. S. (2012) Expert Rev. Anti. Infect. Ther. 10:701-706; Heckbert S. R. et al (1998) J. Trauma 45:545-549; Reynolds H. R. and Hochman J. S., (2008) Circulation 117.686-697). Rather than a specific disease state, shock is a shared pathologic end point arising from disorders such as respiratory failure, hemorrhage, or sepsis that ultimately impair cardiovascular function. For this reason, maintaining a balance between myocardial O₂ supply and demand underlies a central therapeutic framework of critical care medicine.

Of all organs, the heart is metabolically unique both in regard to its energetic demands as well as its O₂ utilization and extraction characteristics. The heart exhibits the highest basal oxygen (O₂) consumption per tissue mass of any organ in the body and is uniquely dependent on aerobic metabolism to sustain contractile function. During acute hypoxic states, the body responds with a compensatory increase in cardiac output that further increases myocardial O₂ demand, predisposing the heart to ischemic stress and myocardial dysfunction. Given its primary physiologic function as a continuous generator of mechanical force, the heart requires an extraordinary supply of biochemical energy and exhibits a far greater rate of ATP turnover than any other organ (Taegtmeyer H. (1994) Curr. Probl. Cardiol. 19:59-113). Furthermore, the heart is exquisitely dependent on aerobic metabolism to meet these high bioenergetic needs, without the ability to derive any meaningful contribution from anaerobic pathways such as glycolysis (Neely J. R. et al. (1972) Prog Cardiomsc. Dis. 15:289-329) This is reflected in the large myocardial volume devoted to mitochondria and the heart's status as the highest O₂ consumer per gram tissue mass of any organ (Taegtmeyer H. (1994) Curr. Prob. Cardiol. 19:59-113; Neely J. R. et al. (1972) Prog. Cardiovasc. Dis. 15:289-329). Importantly, its high O₂ extraction ratio results in lower venous O₂ contents than other tissues, with a significant fraction of cardiomyocytes being exposed to physiologically hypoxic environments at baseline (von Restorff W. et al. (1977) Pflugers Arch. 372:181-185; Walley K. R. et al. (1997) Am. J. Respir. Crit. Care Med. 155: 222-228). When myocardial O₂ supply becomes limited in the face of increased demand, dramatic increases in coronary blood flow as well as cardiomyocyte O₂ extraction attempt to compensate (von Restorff W et al. (1977) Pflugers Arch. 372-181-185; Walley K R. et al. (1997) Am. J. Respir. Crit. Care Med. 155: 222-228; Cain S. M. (1977) J. Appl. Physiol. Respir. Environ. Exerc. Physiol. 42: 228-234). When inadequate, biochemical signs of a switch to anaerobic metabolism are accompanied by an immediate impairment of contractile function (Walley K. R. et al. (1988) Circ. Res. 63:849-859). O₂ consumption is thus vital to provide the biochemical energy required to maintain cardiac mechanical function.

### 5. SUMMARY OF THE INVENTION

In one aspect, provided herein are methods for treating a cardiovascular disorder or pulmonary disorder in a subject in need thereof, said method comprising administering to the subject (a) an H-NOX protein (such as any H-NOX protein or a mixture of H-NOX proteins described herein); and optionally (b) a catecholamine, preferably epinephrine or norepinephrine. In certain embodiments, the cardiovascular disorder or pulmonary disorder is associated with hypoxia.

In one aspect, provided herein are methods for treating a subject undergoing cardiac or respiratory arrest, said method comprising administering to the subject (a) an H-NOX protein (such as any H-NOX protein or a mixture of H-NOX proteins described herein); and optionally (b) a catecholamine, preferably epinephrine or norepinephrine. In one aspect, provided herein are methods for treating a subject undergoing cardiopulmonary resuscitation, said method comprising administering to the subject (a) an H-NOX protein (such as any H-NOX protein or a mixture of H-NOX proteins described herein); and optionally (b) a catecholamine, preferably epinephrine or norepinephrine. In certain embodiments, the subject being treated is hypoxic, has myocardial ischemia, has hemorrhage, or has a trauma. In one embodiment, the subject is hypoxic. In one embodiment, the subject has myocardial ischemia. In one embodiment, the subject has hemorrhage. In one embodiment, the subject has a trauma.

In one aspect, provided herein are methods for treating depressed ventilator function in a subject in need thereof, said method comprising administering to the subject (a) an H-NOX protein (such as any H-NOX protein or a mixture of H-NOX proteins described herein), and optionally (b) a catecholamine, preferably epinephrine or norepinephrine. In one aspect, provided herein are methods for treating anaphylaxis or hemorrhagic shock in a subject in need thereof, said method comprising administering to the subject (a) an H-NOX protein (such as any H-NOX protein or a mixture of H-NOX proteins described herein); and optionally (b) a catecholamine, preferably epinephrine or norepinephrine.

In certain embodiments, provided herein are methods for treating a cardiovascular disorder in a subject in need thereof, said method comprising administering to the subject (a) an H-NOX protein (such as any H-NOX protein or a mixture of H-NOX proteins described herein), and optionally (b) a catecholamine, preferably epinephrine or norepinephrine. In certain embodiments, the cardiovascular disorder is a heart attack, cardiac arrest, catecholamine-induced hypoxemia, impaired cardiovascular function, decreased myocardial function, myocardial hypoxia, or congestive heart failure.

In certain embodiments, provided herein are methods for treating a pulmonary disorder in a subject in need thereof, said method comprising administering to the subject (a) an H-NOX protein (such as any H-NOX protein or a mixture of H-NOX proteins described herein); and optionally (b) a catecholamine, preferably epinephrine or norepinephrine. In certain embodiments, the pulmonary disorder is acute respiratory failure.

In one aspect, provided herein are methods for treating a disorder or condition amenable to treatment with epinephrine or norepinephrine in a subject in need thereof, said method comprising administering to the subject (a) an H-NOX protein (such as any H-NOX protein or a mixture of H-NOX proteins described herein); and optionally (b) a catecholamine, preferably epinephrine or norepinephrine.

In one aspect, provided herein are methods for treating or preventing catecholamine-induced hypoxemia in a subject in need thereof, said method comprising administering to the subject (a) an H-NOX protein (such as any H-NOX protein or a mixture of H-NOX proteins described herein); and optionally (b) a catecholamine, preferably epinephrine or norepinephrine.

In one aspect, provided herein is a pharmaceutical composition comprising (i) an H-NOX protein or a mixture of H-NOX proteins (such as any H-NOX protein or a mixture of H-NOX proteins described herein), and (ii) a catecholamine, preferably epinephrine or norepinephrine. In a specific embodiment, provided herein is an infusion bag comprising a composition comprising (i) an H-NOX protein or a mixture of H-NOX proteins (such as any H-NOX protein or a mixture of H-NOX proteins described herein), and (ii) a catecholamine, preferably epinephrine or norepinephrine.

In certain embodiments, the H-NOX protein used in the compositions and methods described herein is a polymeric H-NOX protein comprising (i) an H-NOX domain of T. tengcongensis H-NOX with an L144F amino acid substitution, and (ii) a polymerization domain.

In certain embodiments, the H-NOX protein used in the compositions and methods described herein is an H-NOX protein that is covalently bound to polyethylene glycol (PEG).

In certain embodiments, the H-NOX protein used in the compositions and methods described herein is a mixture comprising (i) an H-NOX protein covalently bound to polyethylene glycol (PEG), and (ii) an H-NOX protein not bound to PEG. In certain embodiments, administering the H-NOX protein comprises administering a mixture comprising (i) an H-NOX protein covalently bound to polyethylene glycol (PEG), and (ii) an H-NOX protein not bound to PEG. In certain embodiments, the mixture has a weight ratio of the H-NOX protein covalently bound to PEG to the H-NOX protein not bound to PEG of about 9.1, about 8:2, about 7.3, about 6:4, about 1.1, about 4:6, about 3:7, about 2:8, or about 1:9. In one embodiment, the weight ratio of the H-NOX protein covalently bound to PEG to the H-NOX protein not bound to PEG is about 1:1. In certain embodiments, the H-NOX protein covalently bound to PEG and/or the H-NOX protein not bound to PEG is a polymeric H-NOX protein comprising (i) an H-NOX domain of T. tengcongensis H-NOX with an L144F amino acid substitution (e.g., relative to the amino acid sequence of SEQ ID NO:2 set forth herein and (ii) a polymerization domain.

In specific embodiments, the polymeric H-NOX protein used in the compositions and methods described herein comprises a plurality of monomers, wherein each monomer is identical and is a fusion protein comprising the H-NOX domain fused via a peptide linker to the polymerization domain, for example, a trimeric H-NOX wherein the three monomers are each a fusion protein comprising the H-NOX domain fused via a peptide linker to the trimerization domain.

In specific embodiments, the polymeric H-NOX protein used in the compositions and methods described herein is a trimeric H-NOX protein comprising three monomers, wherein each of the monomers comprises the H-NOX domain and a trimerization domain. In one embodiment, the trimerization domain is a foldon domain of bacteriophage T4 fibritin. In one embodiment, the foldon domain has the amino acid sequence of SEQ ID NO:4 herein. In one embodiment, each monomer has the amino acid sequence of SEQ ID NO:8 described herein. In one embodiment, the trimeric H-NOX comprises three PEG molecules per monomer. In one embodiment, the PEG molecule has a molecular weight of 5 kDa. In one embodiment, the PEG molecule is a methoxy PEG.

In one embodiment, the H-NOX protein used in the compositions and methods described herein is OMX-CV. In one embodiment, administering the H-NOX protein comprises administering OMX-CV.

“OMX-CV” as used herein refers to a 1:1 mixture (by weight) of an H-NOX protein covalently bound to polyethylene glycol (PEG) and an H-NOX protein not bound to PEG, wherein the H-NOX protein (both the protein bound to PEG and the protein not bound to PEG) is a trimeric H-NOX protein comprising three monomers, wherein each of the three monomers comprises a T. tengcongensis H-NOX domain covalently linked to a trimerization domain, wherein the trimerization domain is a foldon domain of bacteriophage T4 fibritin (having the amino acid sequence of SEQ ID NO:4 set forth herein), wherein the T. tengcongensis H-NOX domain has an L144F amino acid substitution relative to the amino acid sequence of SEQ ID NO:2 set forth herein, and wherein the trimeric H-NOX protein comprises three PEG molecules per monomer, wherein each of the three PEG molecules is a linear methoxy PEG (m-PEG) having a molecular weight of about 5 kDa, and wherein each of the three monomers has the amino acid sequence of SEQ ID NO:8 set forth herein. As will be understood by a person skilled in the art, the three PEG molecules per monomer is an average number of PEG molecules per monomer.

In certain embodiments, the H-NOX protein is administered before, concurrently with, or after the administration of a catecholamine, preferably epinephrine or norepinephrine. In a specific embodiment, the H-NOX protein is administered within 1 hour, 30 minutes, 15 minutes, 10 minutes, or 5 minutes, of the administration of epinephrine or norepinephrine.

In certain embodiments, the subject being treated in accordance with the methods described herein is a mammal. In one embodiment, the subject is human.

### 6. BRIEF DESCRIPTION OF FIGURES

FIGS. 1A and 1B illustrate an H-NOX trimer and its oxygen-binding characteristics. (A) Ribbon diagrams depicting an H-NOX protein monomer, H-NOX protein trimer, and PEGylated H-NOX protein trimer. The heme cofactor and the bound oxygen are depicted in FIGS. 1A and 1B Models were made using a Tt H-NOX structure (PDB ID 1U4H) and PyMOL (The PyMOL Molecular Graphics System, Version 1.5 Schrödinger, LLC.). (B) Illustration depicting the relative oxygen affinities of hemoglobin, Tt H-NOX (wild type), and OMX-CV overlaid on an oxygen gradient from normoxia to hypoxia. The oxygen affinity of hemoglobin facilitates release of oxygen in peripheral tissues (PO₂ of about 40 mmHg), while the oxygen affinity of OMX-CV facilitates release of oxygen into hypoxic tissues (PO₂ of about 10 mmHg). K_{D}, dissociation constant; mmHg, millimeters mercury; PEG, polyethylene glycol; PO₂, partial pressure of oxygen; Tt, Thermoanaerobacter tengcongensis.

FIGS. 2A-2G show physiologic responses of the cardiovascular system to acute alveolar hypoxia. (A) Schematic of experimental protocol. Physiologic measurements were continuously recorded and logged every second for the duration of the study. At each designated time point, physiologic data were averaged over a 60-second period in 5-second intervals. (B) Average measured PaO₂ in mmHg of all animals (n=13) at baseline (Bsl) compared with 15 minutes following institution of hypoxic ventilation. (C) Average heart rate of all animals at Bsl compared with 15 minutes following institution of hypoxic ventilation. (D) Average mean pulmonary arterial pressure (in mmHg) of all animals at Bsl compared with 15 minutes following institution of hypoxic ventilation. (E) Average mean systemic arterial pressure (in mmHg) of all animals at Bsl compared with 15 minutes following institution of hypoxic ventilation. (F) Average indexed pulmonary vascular resistance (PVR) of all animals at baseline (Bsl) compared with 15 minutes following institution of hypoxic ventilation. PVR of the left lung was calculated as the difference of mean pulmonary arterial pressure and left atrial pressure divided by the indexed LPA blood flow. (G) Average indexed left pulmonary arterial blood flow of all animals at Bsl compared with 15 minutes following institution of hypoxic ventilation. Flow was indexed to body size by dividing by the animal's weight in kilograms. In all figures, “*” denotes significance with p<0.05, while “ns” denotes p>0.05. Error bars demonstrate standard error of the mean, bpm, beats per minute, Bsl, baseline; iLPAQ, indexed left pulmonary artery flow; iLPVR, indexed left pulmonary vascular resistance; LPA, left pulmonary artery; mmHg, millimeters mercury; PA, pulmonary artery; PaO₂, arterial oxygen tension; Veh, vehicle.

FIG. 3 shows cardiac output in control (vehicle-treated) and OMX-CV-treated animals. Indexed left pulmonary arterial blood flow in vehicle-treated versus OMX-CV-treated groups over the duration of the experimental protocol Time 0 represents the physiologic baseline and other time points represent total duration of hypoxic ventilation. Error bars correspond to the standard error of the mean. There is a statistically significant interaction between time and iLPA flow (p<0.05) by two-way ANOVA. There is no significant difference between OMX-CV (n=6) and vehicle (n=7) groups, iLPA, indexed left pulmonary artery; Veh, vehicle.

FIGS. 4A and 4B show systemic vascular resistance (SVR) and PVR before and after OMX-CV and vehicle administration. (A) Indexed PVR in vehicle-treated (n=7) and OMX-CV-treated (n=6) animals during hypoxic ventilation immediately prior to (pre-txt) and following (post-txt) treatment administration. There are no statistically significant differences between groups or within groups pre- and posttreatment. Error bars represent the standard error of the mean. (B) Indexed SVR in vehicle-treated and OMX-CV-treated animals pre-txt and post-txt. There are no statistically significant differences between groups or within groups pre- and posttreatment. Error bars represent the standard error of the mean. Post-txt, immediately following treatment administration, pre-txt, immediately prior to treatment administration; PVR, pulmonary vascular resistance; SVR, systemic vascular resistance; Veh, vehicle.

FIG. 5A-5C show myocardial hypoxia in control (vehicle-treated) and OMX-CV-treated animals. In a subset of vehicle-treated and OMX-CV-treated animals (n=3 each), following measurement of physiologic parameters, pimonidazole was administered intravenously and tissues were collected for analysis 30 minutes later. (A) Quantification of pimonidazole adducts in vehicle-treated and OMX-CV-treated myocardial tissue by pimonidazole ELISA. Values are ±SEM, *p<0.05 by Student t test. (B) Representative images of vehicle-treated and OMX-CV-treated myocardium tissue sections immunostained with antibodies targeting pimonidazole adducts. (C) Representative images of OMX-CV-treated myocardial tissue sections immunostained with antibodies targeting the OMX-CV molecule. Pimo, pimonidazole; Veh, vehicle

FIGS. 6A-6E show the ventricular contractility and circulating catecholamine levels in control (vehicle-treated) and OMX-CV-treated animals. (A) Representative Pressure-Volume loops obtained from the left ventricle of a vehicle-treated animal during transient inferior vena cava (IVC) occlusion Left Ventricle (LV) pressure is measured on the y-axis and LV volume on the x-axis. The superimposed line tangential to the end systolic pressure volume points of each family of loops defines the End Systolic Pressure-Volume Relationship (ESPVR). The family of loops on the left side of FIG. 6A that are closer to the x-axis and their corresponding ESPVR were obtained during the physiologic baseline, while the family of loops on the right side of FIG. 6A that are further away from the x-axis and ESPVR were obtained from the same animal following 1 hour of hypoxic ventilation. (B) Representative Pressure-Volume loops obtained from the LV of an OMX-CV-treated animal during transient inferior vena cava (IVC) occlusion. The family of loops on the left side of FIG. 6B that are closer to the x-axis and their corresponding ESPVR were obtained during the physiologic baseline, while the family of loops on the right side of FIG. 6B that are further away from the x-axis and ESPVR were obtained from the same animal following 1 hour of hypoxic ventilation. (C) Mean right ventricular contractility (as assessed by slope of the ESPVR relative to baseline) in vehicle-treated (n=7) and OMX-CV-treated (n=6) animals after 1 hour of hypoxic ventilation Error bars represent the standard error of the mean, “*” denotes a significant difference between groups with p<0.05. (D) Mean left ventricular contractility (as assessed by slope of the ESPVR relative to baseline) in vehicle-treated (n=7) and OMX-CV-treated (n=6) animals after 1 hour of hypoxic ventilation. Error bars represent the standard error of the mean; “*” denotes a significant difference between groups with p<0.05. (E) Mean serum epinephrine levels (expressed as fold change relative to physiologic baseline) after 1 hour of hypoxic ventilation in vehicle-treated (n=7) and OMX-CV-treated (n=6) animals. Error bars represent the standard error of the mean; “*” denotes a significant difference between groups with p<0.05. (F) Mean serum norepinephrine levels (expressed as fold change relative to physiologic baseline) at 1 hour of hypoxic ventilation in vehicle-treated and OMX-CV-treated animals. Error bars represent the standard error of the mean, “*” denotes a significant difference between groups with p 0.05. Bsln, baseline; ESPVR, end systolic pressure-volume relationship; IVC, inferior vena cava; LV, left ventricle; mmHg, millimeters mercury; RV, right ventricle; Veh, vehicle.

### 7. DETAILED DESCRIPTION

Provided herein are methods for treating any disorder or condition described herein by administering to a subject in need thereof an H-NOX protein (or a mixture of H-NOX proteins), or comprising administering to a subject in need thereof a combination of an H-NOX protein (or a mixture of H-NOX proteins) and a catecholamine (e.g., epinephrine or norepinephrine). Preferably, the catecholamine is epinephrine or norepinephrine.

In one aspect, provided herein are methods for treatment of a cardiovascular disorder or condition in a subject comprising administering to the subject an H-NOX protein (or a mixture of H-NOX proteins), or comprising administering a combination of an H-NOX protein (or a mixture of H-NOX proteins) and a catecholamine (e.g., epinephrine or norepinephrine). In one aspect, provided herein are methods for treatment of a subject undergoing cardiac or respiratory arrest, said method comprising administering to the subject (a) an H-NOX protein (or a mixture of H-NOX proteins), or comprising administering a combination of an H-NOX protein (or a mixture of H-NOX proteins) and a catecholamine (e.g., epinephrine or norepinephrine). In one aspect, provided herein are methods for treatment of a subject undergoing cardiopulmonary resuscitation, said method comprising administering to the subject (a) an H-NOX protein (or a mixture of H-NOX proteins), or comprising administering a combination of an H-NOX protein (or a mixture of H-NOX proteins) and a catecholamine (e.g., epinephrine or norepinephrine). In certain embodiments, provided herein are methods for treating a heart attack or a cardiac arrest in a subject comprising administering to the subject an H-NOX protein (or a mixture of H-NOX proteins), or comprising administering a combination of an H-NOX protein (or a mixture of H-NOX proteins) and a catecholamine (e.g., epinephrine or norepinephrine). In certain embodiments, provided herein are methods for treating depressed ventilator function in a subject comprising administering to the subject an H-NOX protein (or a mixture of H-NOX proteins), or comprising administering a combination of an H-NOX protein (or a mixture of H-NOX proteins) and a catecholamine (e.g., epinephrine or norepinephrine). In certain embodiments, provided herein are methods for treating anaphylaxis or hemorrhagic shock in a subject comprising administering to the subject an H-NOX protein (or a mixture of H-NOX proteins), or comprising administering a combination of an H-NOX protein (or a mixture of H-NOX proteins) and a catecholamine (e.g., epinephrine or norepinephrine).

In certain embodiments, the H-NOX protein (or a mixture of H-NOX proteins) is administered to a subject before, concurrently or after administration of a catecholamine (e.g., epinephrine or norepinephrine). In one embodiment, the H-NOX protein (or a mixture of H-NOX proteins) and a catecholamine (e.g., epinephrine or norepinephrine) are administered concurrently. In a specific embodiment, the 1-1-NOX protein (or mixture of 1-NOX proteins) are administered in the same composition, for example, from the same infusion bag. In a specific embodiment, the H-NOX protein is administered within 24 hours, 12 hours, 1 hour, 30 minutes, 15 minutes, 10 minutes, or 5 minutes, of the administration of epinephrine or norepinephrine.

In certain embodiments of the methods described herein, the subject being treated is hypoxic, has myocardial ischemia, has hemorrhage, or has a trauma. In one embodiment, the subject is hypoxic. In one embodiment, the subject has myocardial ischemia. In one embodiment, the subject has hemorrhage (e.g., has cardiac or pulmonary arrest associated with hemorrhage). In one embodiment, the subject has a trauma (e.g., has cardiac or pulmonary arrest associated with a trauma).

In certain embodiments, the catecholamine used in the compositions and methods described herein is epinephrine, norepinephrine, dopamine, dobutamine, or atropine. In one embodiment, the catecholamine is epinephrine or norepinephrine. In one embodiment, the catecholamine is epinephrine. In one embodiment, the catecholamine is norepinephrine. In one embodiment, the catecholamine is dopamine. In one embodiment, the catecholamine is dobutamine. In one embodiment, the catecholamine is atropine. In one embodiment, atropine is used in the methods described herein, wherein the subject being treated has bradycardia

In a specific embodiment, the H-NOX that is administered in any of the methods described herein is a mixture of H-NOX proteins. In a specific embodiment, the mixture of H-NOX proteins comprises or consists essentially two H-NOX proteins that are identical except that one is PEGylated and one is not PEGylated Preferably, the mixture of H-NOX proteins is OMX-CV.

In certain embodiments, provided herein are methods for treatment of a cardiovascular disorder or condition (e.g., a heart attack or a cardiac arrest) in a subject in need thereof comprising administering (i) a PEGylated H-NOX protein and a non-PEGylated H-NOX protein (such as any of the proteins or mixtures of proteins described below or in International Application Publication No. WO 2017/143104 A1), and optionally (ii) an epinephrine or norepinephrine. In certain embodiments, a mixture comprising a PEGylated H-NOX protein and a non-PEGylated H-NOX protein is administered to a subject before, concurrently or after administration of an epinephrine or norepinephrine. In one embodiment, a mixture comprising a PEGylated H-NOX protein and a non-PEGylated i-NOX protein is administered concurrently (e.g., in one composition, for example, from the same infusion bag) with an epinephrine or norepinephrine.

In one aspect, provided herein are methods for treatment of a pulmonary disorder or condition (such as acute respiratory failure) in a subject in need thereof comprising administering to the subject an H-NOX protein (or a mixture of H-NOX proteins), or comprising administering a combination of an H-NOX protein (or a mixture of H-NOX proteins) and a catecholamine (e.g., epinephrine or norepinephrine). In one aspect, provided herein are methods for treatment or prevention of catecholamine-induced hypoxemia in a subject in need thereof comprising administering to the subject an H-NOX protein (or a mixture of H-NOX proteins), or comprising administering a combination of an H-NOX protein (or a mixture of H-NOX proteins) and a catecholamine (e.g., epinephrine or norepinephrine). In one aspect, provided herein are methods for treatment of a subject in need of or undergoing resuscitation (such as cardiopulmonary resuscitation) comprising administering to the subject an H-NOX protein (or a mixture of H-NOX proteins), or comprising administering a combination of an H-NOX protein (or a mixture of H-NOX proteins) and a catecholamine (e.g., epinephrine or norepinephrine). In certain embodiments, the H-NOX protein (or a mixture of H-NOX proteins) is administered to a subject before, concurrently or after administration of a catecholamine (e.g., epinephrine or norepinephrine). In one embodiment, the H-NOX protein (or a mixture of H-NOX proteins) and a catecholamine (e.g., epinephrine or norepinephrine) are administered concurrently (e.g., in one composition).

In certain embodiments, provided herein are methods for treatment of a pulmonary disorder or condition (such as acute respiratory failure) in a subject in need thereof, for treatment or prevention of catecholamine-induced hypoxemia in a subject in need thereof, or for resuscitation (such as cardiopulmonary resuscitation) of a subject in need thereof, comprising administering an H-NOX protein (or a mixture of H-NOX proteins), or comprising administering a combination of (i) a PEGylated H-NOX protein and a non-PEGylated H-NOX protein (such as any of the proteins or mixtures of proteins described herein or in International Application Publication No. WO 2017/143104 A1), and (ii) an epinephrine or norepinephrine. In certain embodiments, a mixture comprising a PEGylated H-NOX protein and a non-PEGylated H-NOX protein is administered to a subject before, concurrently or after administration of an epinephrine or norepinephrine. In one embodiment, a mixture comprising a PEGylated H-NOX protein and a non-PEGylated H-NOX protein is administered concurrently (e.g., in one composition) with an epinephrine or norepinephrine.

In one aspect, provided herein are compositions comprising a combination of an H-NOX protein (or a mixture of H-NOX proteins) and a catecholamine (e.g., epinephrine or norepinephrine). In certain embodiments, provided herein are compositions comprising a combination of (i) a PEGylated H-NOX protein and a non-PEGylated H-NOX protein (such as any of the proteins or mixtures of proteins described below or in International Application Publication No. WO 2017/143104 A1), and (ii) an epinephrine or norepinephrine.

H-NOX proteins or mixtures of H-NOX proteins, and pharmaceutical compositions of the H-NOX protein or mixtures, that can be used in the compositions and methods provided herein can be any of those described herein or in International Application Publication No. WO 2017/143104 A1, which is incorporated by reference herein in its entirety. For example, page 35, paragraph [0140] to page 61, paragraph [0202] of International Application Publication No. WO 2017/143104 A1, describe H-NOX proteins that can be used in the compositions and methods provided herein and their characteristics. Page 61, paragraph [0203] to page 63, paragraph [0213] of International Application Publication No. WO 2017/143104 A1, describe nucleic acids encoding H-NOX proteins, which nucleic acids can be used for production of H-NOX proteins, which proteins can be used in the compositions and methods provided herein and cells or population of cells containing such nucleic acids. Formulations of H-NOX proteins that can be used in the compositions and methods provided herein are described at, e.g., page 63, paragraph [0214] to page 81, paragraph [0266] of International Application Publication No. WO 2017/143104 A1. Kits with H-NOX proteins that can be used in the practice of the invention provided herein are described at, e.g., page 81, paragraph [0267] to page 84, paragraph [0273] of International Application Publication No. WO 2017/143104 A1. Methods of production of H-NOX proteins that can be used in the practice of the invention provided herein are described at, e.g., page 84, paragraph [0274] to page 84, paragraph [0273] of International Application Publication No. WO 2017/143104 A1. Mixtures comprising a PEGylated H-NOX protein and a non-PEGylated H-NOX protein that can be used in the compositions and methods provided herein are described at, e.g., paragraphs [0226]-[0229], [0228], [0273], [0274] and [0295] of International Application Publication No. WO 2017/143104 A1. The above-mentioned pages and paragraphs of International Application Publication No. WO 2017/143104 A1 are specifically incorporated by reference herein.

In one embodiment, the H-NOX protein used in the compositions and methods provided herein is a polymeric 1-NOX protein, wherein each monomer comprises (i) an 11-NOX domain of T. tengcongensis H-NOX (e.g., with an 144F amino acid substitution relative to the amino acid sequence of SEQ ID NO:2 set forth herein or in International Application Publication No. WO 2017/143104 A1, and (ii) a polymerization domain. In a preferred embodiment, the polymeric H-NOX protein is a trimeric H-NOX protein comprising three monomers, wherein each monomer comprises (i) an H-NOX domain of T. tengcongensis H-NOX with an L144F amino acid substitution relative to the amino acid sequence of SEQ ID NO 2 set forth herein or in International Application Publication No. WO 2017/143104 A1, and (ii) a trimerization domain (such as a foldon domain of bacteriophage T4 fibritin, e.g., having the amino acid sequence of SEQ ID NO:4 set forth herein or in International Application Publication No. WO 2017/143104 A1). Preferably, the polymeric (preferably trimeric) H-NOX protein comprises monomers, wherein each monomer is a fusion protein comprising the H-NOX domain fused via a peptide linker to the polymerization (preferably trimerization) domain. The peptide linker can be any of the amino acid linkers as described herein or in International Application Publication No. WO 2017/143104 A1 (see, e.g., paragraphs [0095], [0142], [0169], [0173], [0177], and [0178] of International Application Publication No. WO 2017/143104 A1, which are specifically incorporated by reference herein).

In one embodiment, the mixture of H-NOX proteins used in the compositions and methods provided herein is a mixture of an H-NOX protein covalently bound to polyethylene glycol (PEG) and an H-NOX protein not bound to PEG, wherein the H-NOX protein is a polymeric, e.g., trimeric H-NOX protein described herein or in International Application Publication No. WO 2017/143104 A1. In one embodiment, the mixture of H-NOX proteins used in the compositions and methods provided herein comprises the ratio of the H-NOX protein covalently bound to PEG to the H-NOX protein not bound to PEG of about 9:1, about 8:2, about 7:3, about 6:4, about 1.1, about 4:6, about 3:7, about 2:8, or about 1:9. In a preferred, embodiment, the ratio of the H-NOX protein covalently bound to PEG to the H-NOX protein not bound to PEG is about 1:1.

In certain embodiments, the H-NOX protein used in the compositions and methods provided herein is a polymeric H-NOX protein (e.g., a trimeric H-NOX protein) comprising one, two, three, four, five, six, or seven PEG molecules per monomer. In a preferred embodiment, the H-NOX protein used in the compositions and methods provided herein is a polymeric H-NOX protein (e.g., a trimeric H-NOX protein) comprising three PEG molecules per monomer. In certain embodiments, the PEG molecule has a molecular weight between 1 kDa and 10 kDa, or between 5 kDa and 10 kDa. In a preferred embodiment, the PEG molecule has a molecular weight of 5 kDa. In one embodiment, the PEG molecule is a linear methoxy PEG (m-PEG). In one embodiment, the H-NOX protein used in the compositions and methods provided herein is a polymeric H-NOX protein (e.g., a trimeric H-NOX protein) comprising three PEG molecules per monomer, wherein each of the PEG molecule has a molecular weight of 5 kDa and, optionally, wherein each of the PEG molecules is a linear methoxy PEG (m-PEG).

In specific embodiments, a PEGylated H-NOX protein and a non-PEGylated H-NOX protein can be administered simultaneously, sequentially or as a mixture, as described herein or in International Application Publication No. WO 2017/143104 A1 (see, e.g., claims 17-25 of International Application Publication No WO 2017/143104 A1, which are specifically incorporated by reference herein).

Doses of an H-NOX protein (or a mixture of H-NOX proteins) and dosage regimens that can be used in the compositions and methods provided herein can be determined by the treating physician, and include those described herein or in International Application Publication No. WO 2017/143104 A1, e.g., at paragraphs [0259]-[0261] In one embodiment, an H-NOX protein (or a mixture of H-NOX proteins) described herein or in International Application Publication No. WO 2017/143104 A1 is administered at the following dosage regimen: about 200 mg/kg bolus (e.g., over 10 min), followed by continuous infusion at about 70 mg/kg/h. Dosing frequencies of an H-NOX protein (or a mixture of H-NOX proteins) that can be used in the compositions and methods provided herein are described herein or in International Application Publication No. WO 2017/143104 A1, e.g., at paragraphs [0262]-[0264]. Routes of administration of an H-NOX protein (or a mixture of H-NOX proteins) that can be used in the methods provided herein are described herein or in International Application Publication No. WO 2017/143104 A1, e.g., at paragraphs [0037], [0047], [0216], [0232], [0233], [0257], [0258], and [0263]. In certain embodiments, an H-NOX protein (or a mixture of H-NOX proteins) is administered intravenously, subcutaneously, intramuscularly, intracardially, or endotracheally. In one embodiment, an H-NOX protein (or a mixture of H-NOX proteins) is administered intravenously. The above-mentioned paragraphs of International Application Publication No. WO 2017/143104 A1 are specifically incorporated by reference herein.

Doses, dosage regimens and modes of administration of catecholamines (such as epinephrine or norepinephrine) that can be used for the clinical indications described herein are known in the art. In one embodiment, epinephrine is used in the compositions and methods provided herein in an amount from 0.1 mg to 2 mg, from 0.2 mg to 1 mg, or from 0.5 mg to 1 mg, or infused in an amount from 0.05 to 2 mcg/kg/min, or from 0.1 to 0.5 mcg/kg/min. In one embodiment, epinephrine is used in the compositions and methods provided herein in an amount from 0.5 to 1.5 mg (e.g., 1 mg), for example, for intravenous administration every 3-5 minutes (e.g., for the treatment of a human adult). In one embodiment, epinephrine is administered in an amount from 0.01 to 0.03 mg/kg (e.g., for the treatment of a human child). In one embodiment, epinephrine is infused (e.g., as a continuous intravenous drip) in an amount from 2 to 10 mcg/min (e.g., wherein the subject being treated has bradycardia). In one embodiment, epinephrine is infused in an amount from 0.1 to 0.5 mcg/kg/min (e.g., wherein the subject being treated has hypotension following cardiac or pulmonary arrest). In one embodiment, atropine is used in the compositions and methods provided herein in an amount from 0.25 to 1 mg (e.g., 0.5 mg), for example, for intravenous administration every 3-5 minutes (e.g., for the treatment of a human adult). In one embodiment, atropine is administered an amount from 0.01 to 0.05 mg/kg (e.g., 0.02 mg/kg), for example, intravenously every 3-5 minutes (e.g., for the treatment of a human child). In one embodiment, norepinephrine is infused in an amount from 0.1 to 3.3 mcg/kg/min, from 0.1 to 1.5 mcg/kg/min, from 0.2 to 1.3 mcg/kg/min, or from 0.1 to 0.5 mcg/kg/min. In certain embodiments, a catecholamine (e.g., epinephrine or norepinephrine) is administered intravenously, subcutaneously, intramuscularly, intracardially, or endotracheally. In one embodiment, a catecholamine (e.g., epinephrine or norepinephrine) is administered intravenously.

The conditions and disorders that can be treated in accordance with the methods described herein include, without limitation, heart attack, cardiac arrest, acute respiratory failure, catecholamine-induced hypoxemia, impaired cardiovascular function, decreased myocardial function (e.g., decreased myocardial contractility), and myocardial hypoxia. The conditions and disorders that can be treated in accordance with the methods described herein also include, without limitation, depressed ventilator function, anaphylaxis, and hemorrhagic shock. In certain embodiments, cardiovascular conditions are treated in accordance with the methods described herein, such as cardiovascular conditions associated with hypoxia (e.g., hypoxic stress). In one embodiment, provided herein are methods for treating a subject undergoing cardiac or respiratory arrest (e.g., cardiac or respiratory arrest associated with hypoxia). In one embodiment, provided herein are methods for treating a subject having a heart attack or a cardiac arrest. In certain embodiments, pulmonary conditions are treated in accordance with the methods described herein, such as pulmonary conditions associated with hypoxia. In one embodiment, provided herein are methods for treating a subject having acute respiratory failure. In one embodiment, provided herein are methods for treating a subject that has undergone or will undergo treatment with a catecholamine (such as epinephrine or norepinephrine). In one embodiment, provided herein are methods for treating or preventing a catecholamine-induced hypoxemia (e.g., epinephrine-induced hypoxemia or norepinephrine-induced hypoxemia) by administering an H-NOX protein (or a mixture of proteins) before, in conjunction with or after the administration of a catecholamine (e.g., epinephrine or norepinephrine). In certain embodiments, provided herein are methods for treating a subject in need of cardiopulmonary resuscitation. In one embodiment, provided herein are methods for treating a subject undergoing cardiopulmonary resuscitation. When a subject is undergoing cardiopulmonary resuscitation, the subject is typically systemically hypoxic; it is believed that the acute coadministration of H-NOX protein with a catecholamine (e.g., epinephrine) may aid the heart's ability to respond to epinephrine. In one embodiment, provided herein are methods for treating a subject in need of or undergoing cardiopulmonary resuscitation, wherein the need for resuscitation is associated with hemorrhage in the subject. In one embodiment, provided herein are methods for treating a subject in need of or undergoing cardiopulmonary resuscitation, wherein the need for resuscitation is associated with trauma in the subject.

In certain embodiments, the subject treated using the compositions and methods described herein is a mammal. In a preferred embodiment, the mammal is a human. In one embodiment, the human is an adult. In one embodiment, the human is a child (e.g., under the age of 12).

### 7.1. Terminology

As used herein, the term “about” comprises the specified value plus or minus 1, 2, 3, 4, 5, 6, 7, 8, 9, 10, 11, 12, 13, 14, or 15% of the specified value.

It is understood that aspects and embodiments of the invention described herein include “comprising,” “consisting,” and “consisting essentially of” aspects and embodiments.

The terms “polypeptide” and “protein” are used interchangeably to refer to a polymer of amino acid residues, and are not limited to a minimum length. Such polymers of amino acid residues may contain natural or non-natural amino acid residues, and include, but are not limited to, peptides, oligopeptides, dimers, trimers, and polymers of amino acid residues. Both full-length proteins and fragments thereof are encompassed by the definition. The terms also include post-expression modifications of the polypeptide, for example, glycosylation, sialylation, acetylation, phosphorylation, and the like. Furthermore, for purposes of the present invention, a “polypeptide” refers to a protein which includes modifications, such as deletions, additions, and substitutions (generally conservative in nature), to the native sequence, as long as the protein maintains the desired activity. These modifications may be deliberate, as through site-directed mutagenesis, or may be accidental, such as through mutations of hosts which produce the proteins or errors due to PCR amplification. As used herein, a protein may include two or more subunits, covalently or non-covalently associated; for example, a protein may include two or more associated monomers.

The terms “nucleic acid molecule”, “nucleic acid” and “polynucleotide” may be used interchangeably, and refer to a polymer of nucleotides Such polymers of nucleotides may contain natural and/or non-natural nucleotides, and include, but are not limited to, DNA, RNA, and PNA. “Nucleic acid sequence” refers to the linear sequence of nucleotides that comprise the nucleic acid molecule or polynucleotide.

As used herein, an “H-NOX protein” means a protein that has an H-NOX domain (named for Heme-Nitric oxide and Oxygen binding domain). An H-NOX protein may or may not contain one or more other domains in addition to the H-NOX domain. In some examples, an H-NOX protein does not comprise a guanylyl cyclase domain. An H-NOX protein may or may not comprise a polymerization domain.

As used herein, a “polymeric H-NOX protein” is an H-NOX protein comprising two or more H-NOX domains. The H-NOX domains may be covalently or non-covalently associated.

As used herein, an “H-NOX domain” is all or a portion of a protein that binds nitric oxide and/or oxygen by way of heme. The H-NOX domain may comprise heme or may be found as an apoproprotein that is capable of binding heme. In some examples, an H-NOX domain includes six alpha-helices, followed by two beta-strands, followed by one alpha-helix, followed by two beta strands. In some examples, an H-NOX domain corresponds to the H-NOX domain of Thermoanaerobacter tengcongensis H-NOX set forth in SEQ ID NO:2. For example, the H-NOX domain may be at least about 10%, 15%, 20%, 25%, 30%, 40%, 50%, 60%, 70%, 80%, 90%, 95%, or 99% identical to the H-NOX domain of Thermoanaerobacter tengcongensis H-NOX set forth in SEQ ID NO:2. In some embodiments, the H-NOX domain may be 10%-20%, 20%-30%, 30%-40%, 40%-50%, 50%-60%, 60%-70%, 70%-80%, 80%-90%, 90%-95%, 95%-99% or 100% identical to the H-NOX domain of Thermoanaerobacter tengcongensis H-NOX set forth in SEQ ID NO:2.

As used herein, a “polymerization domain” is a domain (e.g. a polypeptide domain) that promotes the association of monomeric moieties to form a polymeric structure. For example, a polymerization domain may promote the association of monomeric H-NOX domains to generate a polymeric H-NOX protein. An exemplary polymerization domain is the foldon domain of T4 bacteriophage, which promotes the formation of trimeric polypeptides. Other examples of polymerization domains include, but are not limited to, Arc, POZ, coiled coil domains (including GCN4, leucine zippers, Velcro), uteroglobin, collagen, 3-stranded coiled colis (matrilin-1), thrombosporins, TRPV1-C, P53, Mnt, avadin, streptavidin, Bcr-Abl, COMP, verotoxin subunit B, CamKII, RCK, and domains from N ethylmaleimide-sensitive fusion protein, STM3548, KaiC, TyrR, Hcp1, CcmK4, GP41, anthrax protective antigen, aerolysin, a-hemolysin, C4b-binding protein, Mi-CK, arylsurfatase A, and viral capsid proteins.

As used herein, an “amino acid linker sequence” or an “amino acid spacer sequence” is a short polypeptide sequence that may be used to link two domains of a protein. In some embodiments, the amino acid linker sequence is one, two, three, four, five, six, seven, eight, nine, ten or more than ten amino acids in length. Exemplary amino acid linker sequences include but are not limited to a Gly-Ser-Gly sequence and an Arg-Gly-Ser sequence.

As used herein, a “His6 tag” refers to a peptide comprising six His residues attached to a polypeptide. A His6 tag may be used to facilitate protein purification; for example, using chromatography specific for the His6 tag. Following purification, the His6 tag may be cleaved using an exopeptidase.

A “native sequence” polypeptide comprises a polypeptide having the same amino acid sequence as a polypeptide found in nature. Thus, a native sequence polypeptide can have the amino acid sequence of naturally occurring polypeptide from any organism. Such native sequence polypeptide can be isolated from nature or can be produced by recombinant or synthetic means. The term “native sequence” polypeptide specifically encompasses naturally occurring truncated or secreted forms of the polypeptide (e.g., an extracellular domain sequence), naturally occurring variant forms (e.g., alternatively spliced forms) and naturally occurring allelic variants of the polypeptide.

A polypeptide “variant” means a biologically active polypeptide having at least about 80% amino acid sequence identity with the native sequence polypeptide after aligning the sequences and introducing gaps, if necessary, to achieve the maximum percent sequence identity, and not considering any conservative substitutions as part of the sequence identity. Such variants include, for instance, polypeptides wherein one or more amino acid residues are added, or deleted, at the N- or C-terminus of the polypeptide. In some embodiments, a variant will have at least about any one of 80%, 90% or 95% amino acid sequence identity with the native sequence polypeptide. In some embodiments, a variant will have about any one of 80%-90%, 90%-95% or 95%-99% amino acid sequence identity with the native sequence polypeptide.

As used herein, a “mutant protein” means a protein with one or more mutations compared to a protein occurring in nature. In one embodiment, the mutant protein has a sequence that differs from that of all proteins occurring in nature. In various embodiments, the amino acid sequence of the mutant protein is at least about any of 10, 15, 20, 25, 30, 40, 50, 60, 70, 80, 90, 95, 97, 98, 99, or 99.5% identical to that of the corresponding region of a protein occurring in nature. In some embodiments, the amino acid sequence of the mutant protein is at least about any of 10%-20%, 20%-30%, 30%-40%, 40%-50%, 50%-60%, 60%-70%, 70%-80%, 80%-90%, 90%-95%, 95%-99% or 100% identical to that of the corresponding region of a protein occurring in nature. In some embodiments, the mutant protein is a protein fragment that contains at least about any of 25, 50, 75, 100, 150, 200, 300, or 400 contiguous amino acids from a full-length protein. In some embodiments, the mutant protein is a protein fragment that contains about any of 25-50, 50-75, 75-100, 100-150, 150-200, 200-300, or 300-400 contiguous amino acids from a full-length protein. Sequence identity can be measured, for example, using sequence analysis software with the default parameters specified therein (e.g., Sequence Analysis Software Package of the Genetics Computer Group, University of Wisconsin Biotechnology Center, 1710 University Avenue, Madison, Wis. 53705). This software program matches similar sequences by assigning degrees of homology to various amino acids replacements, deletions, and other modifications.

As used herein, a “mutation” means an alteration in a reference nucleic acid or amino acid sequence occurring in nature. Exemplary nucleic acid mutations include an insertion, deletion, frameshift mutation, silent mutation, nonsense mutation, or missense mutation. In some embodiments, the nucleic acid mutation is not a silent mutation. Exemplary protein mutations include the insertion of one or more amino acids (e.g., the insertion of 2, 3, 4, 5, 6, 7, 8, 9, or 10 amino acids), the deletion of one or more amino acids (e.g., a deletion of N-terminal, C-terminal, and/or internal residues, such as the deletion of at least about any of 5, 10, 15, 25, 50, 75, 100, 150, 200, 300, or more amino acids or a deletion of about any of 5-10, 10-15, 15-25, 25-50, 50-75, 75-100, 100-150, 150-200, 200-300, or 300-400 amino acids), the replacement of one or more amino acids (e.g., the replacement of 1, 2, 3, 4, 5, 6, 7, 8, 9, or 10 amino acids), or combinations of two or more of the foregoing. The nomenclature used in referring to a particular amino acid mutation first identifies the wild-type amino acid, followed by the residue number and finally the substitute amino acid. For example, Y140L means that tyrosine has been replaced by a leucine at residue number 140. Likewise, a variant H-NOX protein may be referred to by the amino acid variations of the H-NOX protein. For example, a T. tengcongensis Y140L H-NOX protein refers to a T. tengcongensis H-NOX protein in which the tyrosine residue at position number 140 has been replaced by a leucine residue and a T. tengcongensis W9F/Y140L H-NOX protein refers to a T. tengcongensis H-NOX protein in which the tryptophan residue at position 9 has been replaced by a phenylalanine residue and the tyrosine residue at position number 140 has been replaced by a leucine residue.

An “evolutionary conserved mutation” is the replacement of an amino acid in one protein by an amino acid in the corresponding position of another protein in the same protein family.

As used herein, “derived from” refers to the source of the protein into which one or more mutations is introduced. For example, a protein that is “derived from a mammalian protein” refers to protein of interest that results from introducing one or more mutations into the sequence of a wild-type (i.e., a sequence occurring in nature) mammalian protein.

As used herein, “Percent (%) amino acid sequence identity” and “homology” with respect to a peptide, polypeptide or antibody sequence are defined as the percentage of amino acid residues in a candidate sequence that are identical with the amino acid residues in the specific peptide or polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or MEGALIGN™ (DNASTAR) software. Those skilled in the art can determine appropriate parameters for measuring alignment, including any algorithms needed to achieve maximal alignment over the full length of the sequences being compared.

As used herein, a “koff” refers to a dissociation rate, such as the rate of release of O₂ or NO from a protein. A lower numerical lower koff indicates a slower rate of dissociation.

As used herein, “kon” refers to an association rate, such as the rate of binding of O₂ or NO to a protein. A lower numerical lower kon indicates a slower rate of association.

As used herein, “dissociation constant” refers to a “kinetic dissociation constant” or a “calculated dissociation constant.” A “kinetic dissociation constant” or “KD” is a ratio of kinetic off-rate (koff) to kinetic on-rate (kon), such as a Ku value determined as an absolute value using standard methods (e.g., standard spectroscopic, stopped-flow, or flash-photolysis methods) including methods known to the skilled artisan and/or described herein. “Calculated dissociation constant” or “calculated KD” refers to an approximation of the kinetic dissociation constant based on a measured koff. A value for the kon is derived via the correlation between kinetic K_{D} and koff as described herein.

As used herein, “oxygen affinity” is a qualitative term that refers to the strength of oxygen binding to the heme moiety of a protein. This affinity is affected by both the koff and kon for oxygen. A numerically lower oxygen KD value means a higher affinity.

As used herein, “NO affinity” is a qualitative term that refers to the strength of NO binding to a protein (such as binding to a heme group or to an oxygen bound to a heme group associated with a protein). This affinity is affected by both the koff and kon for NO. A numerically lower NO KD value means a higher affinity.

As used herein, “NO stability” refers to the stability or resistance of a protein to oxidation by NO in the presence of oxygen. For example, the ability of the protein to not be oxidized when bound to NO in the presence of oxygen is indicative of the protein's NO stability. In some embodiments, less than about any of 50, 40, 30, 10, or 5% of an H-NOX protein is oxidized after incubation for about any of 1, 2, 4, 6, 8, 10, 15, or 20 hours at 20° C.

As used herein, “NO reactivity” refers to the rate at which iron in the heme of a heme-binding protein is oxidized by NO in the presence of oxygen. A lower numerical value for NO reactivity in units of s−1 indicates a lower NO reactivity

As used herein, an “autoxidation rate” refers to the rate at which iron in the heme of a heme-binding protein is autoxidized. A lower numerical autoxidation rate in units of s−1 indicates a lower autoxidation rate.

The term “vector” is used to describe a polynucleotide that may be engineered to contain a cloned polynucleotide or polynucleotides that may be propagated in a host cell. A vector may include one or more of the following elements, an origin of replication, one or more regulatory sequences (such as, for example, promoters and/or enhancers) that regulate the expression of the polypeptide of interest, and/or one or more selectable marker genes (such as, for example, antibiotic resistance genes and genes that may be used in colorimetric assays, e.g., β-galactosidase). The term “expression vector” refers to a vector that is used to express a polypeptide of interest in a host cell.

A “host cell” refers to a cell that may be or has been a recipient of a vector or isolated polynucleotide. Host cells may be prokaryotic cells or eukaryotic cells Exemplary eukaryotic cells include mammalian cells, such as primate or non-primate animal cells; fungal cells, such as yeast; plant cells, and insect cells. Exemplary prokaryotic cells include bacterial cells; for example, E. coli cells.

The term “isolated” as used herein refers to a molecule that has been separated from at least some of the components with which it is typically found in nature or produced. For example, a polypeptide is referred to as “isolated” when it is separated from at least some of the components of the cell in which it was produced. Where a polypeptide is secreted by a cell after expression, physically separating the supernatant containing the polypeptide from the cell that produced it is considered to be “isolating” the polypeptide. Similarly, a polynucleotide is referred to as “isolated” when it is not part of the larger polynucleotide (such as, for example, genomic DNA or mitochondrial DNA, in the case of a DNA polynucleotide) in which it is typically found in nature, or is separated from at least some of the components of the cell in which it was produced, e.g., in the case of an RNA polynucleotide. Thus, a DNA polynucleotide that is contained in a vector inside a host cell may be referred to as “isolated.”

“OMX-CV” as used herein refers to a 1:1 mixture (by weight) of an H-NOX protein covalently bound to polyethylene glycol (PEG) and an H-NOX protein not bound to PEG, wherein the H-NOX protein (both the protein bound to PEG and the protein not bound to PEG) is a trimeric H-NOX protein comprising three monomers, wherein each of the three monomers comprises a T. tengcongensis H-NOX domain covalently linked to a trimerization domain, wherein the trimerization domain is a foldon domain of bacteriophage T4 fibritin (having the amino acid sequence of SEQ ID NO:4 set forth herein), wherein the T. tengcongensis H-NOX domain has an L144F amino acid substitution relative to the amino acid sequence of SEQ ID NO:2 set forth herein, and wherein the trimeric H-NOX protein comprises three PEG molecules per monomer, wherein each of the three PEG molecules is a linear methoxy PEG (m-PEG) having a molecular weight of about 5 kDa, and wherein each of the three monomers has the amino acid sequence of SEQ ID NO:8 set forth herein. As will be understood by a person skilled in the art, the three PEG molecules per monomer is an average number of PEG molecules per monomer.

The terms “individual” or “subject” are used interchangeably herein to refer to an animal; for example a mammal. In some embodiments, methods of treating mammals, including, but not limited to, humans, rodents, simians, felines, canines, equines, bovines, porcines, ovines, caprines, mammalian laboratory animals, mammalian farm animals, mammalian sport animals, and mammalian pets, are provided. In some examples, an “individual” or “subject” refers to an individual or subject in need of treatment for a disease or disorder.

A “disease” or “disorder” as used herein refers to a condition where treatment is needed

As used herein, the term “hypoxic penumbra” refers to the area surrounding an injury where blood flow, and therefore oxygen transport is reduced locally, leading to hypoxia of the cells near the location of the original insult. This lack of oxygen can lead to hypoxic cell death (infarction) and amplify the original damage from the injury.

As used herein, the term “hypoperfusion” refers to an inadequate supply of blood to an organ or extremity (e.g., the brain, the heart, or the lungs). If hypoperfusion persists, it can cause hypoxia and can deprive the tissue of needed nutrients, oxygen, and waste disposal. In some examples hypoperfusion can cause brain tissue death and long-term neurological dysfunction.

As used herein, “treatment” is an approach for obtaining beneficial or desired clinical results. “Treatment” as used herein, covers any administration or application of a therapeutic for disease in a mammal, including a human. For purposes of this invention, beneficial or desired clinical results include, but are not limited to, any one or more of: alleviation of one or more symptoms, diminishment of extent of disease, preventing or delaying spread (e.g., reducing infarct in a hypoxic penumbra associated with organ injury) of disease, preventing or delaying recurrence of disease, delay or slowing of disease progression, amelioration of the disease state, inhibiting the disease or progression of the disease, inhibiting or slowing the disease or its progression, arresting its development, and remission (whether partial or total). Also encompassed by “treatment” is a reduction of pathological consequence of a proliferative disease (e.g., the pathological consequences of sustained hypoperfusion of a tissue). The methods of the invention contemplate any one or more of these aspects of treatment.

The terms “inhibition” or “inhibit” refer to a decrease or cessation of any phenotypic characteristic or to the decrease or cessation in the incidence, degree, or likelihood of that characteristic. To “reduce” or “inhibit” is to decrease, reduce or arrest an activity, function, and/or amount as compared to a reference. In certain embodiments, by “reduce” or “inhibit” is meant the ability to cause an overall decrease of 20% or greater. In another embodiment, by “reduce” or “inhibit” is meant the ability to cause an overall decrease of 50% or greater. In yet another embodiment, by “reduce” or “inhibit” is meant the ability to cause an overall decrease of 75%, 85%, 90%, 95%, or 99%.

As used herein, “delaying development of a disease” means to defer, hinder, slow, retard, stabilize, suppress and/or postpone development of the disease or disorder (such as tissue hypoxia related diseases and disorders). This delay can be of varying lengths of time, depending on the history of the disease and/or individual being treated. As is evident to one skilled in the art, a sufficient or significant delay can, in effect, encompass prevention, in that the individual does not develop the disease. For example, a late stage cancer, such as development of metastasis, may be delayed.

A “reference” as used herein, refers to any sample, standard, or level that is used for comparison purposes. A reference may be obtained from a healthy and/or non-diseased sample. In some examples, a reference may be obtained from an untreated sample. In some examples, a reference is obtained from a non-diseased on non-treated sample of a subject individual. In some examples, a reference is obtained from one or more healthy individuals who are not the subject or patient.

“Preventing,” as used herein, includes providing prophylaxis with respect to the occurrence or recurrence of a disease in a subject that may be predisposed to the disease but has not yet been diagnosed with the disease.

An “effective amount” of an agent refers to an amount effective, at dosages and for periods of time necessary, to achieve the desired therapeutic or prophylactic result.

A “therapeutically effective amount” of a substance/molecule of the invention, agonist or antagonist may vary according to factors such as the disease state, age, sex, and weight of the individual, and the ability of the substance/molecule, agonist or antagonist to elicit a desired response in the individual. A therapeutically effective amount is also one in which any toxic or detrimental effects of the substance/molecule, agonist or antagonist are outweighed by the therapeutically beneficial effects. A therapeutically effective amount may be delivered in one or more administrations.

The term “pharmaceutical composition” refer to a preparation which is in such form as to permit the biological activity of the active ingredient(s) to be effective, and which contains no additional components which are unacceptably toxic to a subject to which the formulation would be administered. Such formulations may be sterile and essentially free of endotoxins.

A “pharmaceutically acceptable carrier” refers to a non-toxic solid, semisolid, or liquid filler, diluent, encapsulating material, formulation auxiliary, or carrier conventional in the art for use with a therapeutic agent that together comprise a “pharmaceutical composition” for administration to a subject. A pharmaceutically acceptable carrier is non-toxic to recipients at the dosages and concentrations employed and is compatible with other ingredients of the formulation. The pharmaceutically acceptable carrier is appropriate for the formulation employed.

A “sterile” formulation is aseptic or essentially free from living microorganisms and their spores.

Administration “in combination with” one or more further therapeutic agents includes simultaneous (concurrent) and consecutive or sequential administration in any order.

The term “concurrently” or “simultaneously” is used herein to refer to administration of two or more therapeutic agents, where at least part of the administration overlaps in time or where the administration of one therapeutic agent falls within a short period of time relative to administration of the other therapeutic agent. For example, the two or more therapeutic agents are administered with a time separation of no more than about 1 day, such as no more than about any of 60, 30, 15, 10, 5, or 1 minutes.

The term “sequentially” is used herein to refer to administration of two or more therapeutic agents where the administration of one or more agent(s) continues after discontinuing the administration of one or more other agent(s). For example, administration of the two or more therapeutic agents are administered with a time separation of more than about 15 minutes, such as about any of 20, 30, 40, 50, or 60 minutes, 1 day, 2 days, 3 days, 1 week, 2 weeks, or 1 month.

As used herein, “in conjunction with” refers to administration of one treatment modality in addition to another treatment modality. As such, “in conjunction with” refers to administration of one treatment modality before, during or after administration of the other treatment modality to the individual.

The term “package insert” is used to refer to instructions customarily included in commercial packages of therapeutic products, that contain information about the indications, usage, dosage, administration, combination therapy, contraindications and/or warnings concerning the use of such therapeutic products.

An “article of manufacture” is any manufacture (e.g., a package or container) or kit comprising at least one reagent, e.g., a medicament for treatment of a disease or disorder (e.g., a hypoxia related disease or disorder), or a probe for specifically detecting a biomarker described herein. In certain embodiments, the manufacture or kit is promoted, distributed, or sold as a unit for performing the methods described herein.

### 7.2 H-NOX Proteins

### 7.2.1 Overview of H-NOX Protein Family

Unless otherwise indicated, any wild-type or mutant H-NOX protein can be used in the compositions, kits, and methods as described herein. As used herein, an “H-NOX protein” means a protein that has an H-NOX domain (named for Heme-Nitric oxide and OXygen binding domain). An H-NOX protein may or may not contain one or more other domains in addition to the H-NOX domain. H-NOX proteins are members of a highly-conserved, well-characterized family of hemoproteins (Iyer, L. M. et al. (Feb. 3, 2003) BMC Genomics 4(1):5; Karow, D. S. et al. (Aug. 10, 2004) Biochemistry 43(31):10203-10211; Boon, E. M. et al. (2005) Nature Chem. Biol. 1:53-59; Boon, E. M. et al. (October 2005) Curr. Opin. Chem. Biol. 9(5):441-446; Boon, E. M. et al. (2005) J. Inorg. Biochem. 99(4):892-902). H-NOX proteins are also referred to as Pfam 07700 proteins or HNOB proteins (Pfam—A database of protein domain family alignments and Hidden Markov Models, Copyright (C) 1996-2006 The Pfam Consortium; GNU LGPL Free Software Foundation, Inc., 59 Temple Place—Suite 330, Boston, Mass. 02111-1307, USA). In some embodiments, an H-NOX protein has, or is predicted to have, a secondary structure that includes six alpha-helices, followed by two beta-strands, followed by one alpha-helix, followed by two beta-strands. An H-NOX protein can be an apoprotein that is capable of binding heme or a holoprotein with heme bound. An H-NOX protein can covalently or non-covalently bind a heme group. Some H-NOX proteins bind NO but not O₂, and others bind both NO and O₂. H-NOX domains from facultative aerobes that have been isolated bind NO but not O₂. H-NOX proteins from obligate aerobic prokaryotes, C. elegans, and D. melanogaster bind NO and O₂. Mammals have two H-NOX proteins: β1 and β2. An alignment of mouse, rat, cow, and human H-NOX sequences shows that these species share >99% identity. In some embodiments, the H-NOX domain of an H-NOX protein or the entire H-NOX protein is at least about any of 10, 15, 20, 25, 30, 40, 50, 60, 70, 80, 90, 95, 97, 98, 99, or 99.5% identical to that of the corresponding region of a naturally-occurring Thermoanaerobacter tengcongensis H-NOX protein (e.g. SEQ ID NO:2) or a naturally-occurring sGC protein (e.g., a naturally-occurring sGC β1 protein). In some embodiments, the H-NOX domain of an H-NOX protein or the entire H-NOX protein is at least about any of 10-20%, 20-30%, 30-40%, 40-50%, 5060%, 60-70%, 70-80%, 80-90%, 90-95%, 95-99, or 99-99.9% identical to that of the corresponding region of a naturally-occurring Thermoanaerobacter tengcongensis 1-NOX protein (e.g. SEQ ID NO:2) or a naturally-occurring sGC protein (e.g., a naturally-occurring sGC β1 protein) As discussed further herein, an H-NOX protein may optionally contain one or more mutations relative to the corresponding naturally-occurring H-NOX protein. In some embodiments, the H-NOX protein includes one or more domains in addition to the H-NOX domain. In particular embodiments, the H-NOX protein includes one or more domains or the entire sequence from another protein. For example, the H-NOX protein may be a fusion protein that includes an H-NOX domain and part or all of another protein, such as albumin (e.g., human serum albumin). In some embodiments, only the H-NOX domain is present. In some embodiments, the H-NOX protein does not comprise a guanylyl cyclase domain. In some embodiments, the H-NOX protein comprises a tag; for example, a His₆ tag.

### 7.2.2 Polymeric H-NOX Proteins

In some aspects, the invention provides polymeric H-NOX proteins comprising two or more H-NOX domains. The two or more H-NOX domains may be covalently linked or noncovalently linked. In some embodiments, the polymeric H-NOX protein is in the form of a dimer, a trimer, a tetramer, a pentamer, a hexamer, a heptamer, an octomer, a nanomer, or a decamer. In some embodiments, the polymeric H-NOX protein comprises homologous H-NOX domains. In some embodiments, the polymeric H-NOX protein comprises heterologous H-NOX domains; for example, the H-NOX domains may comprises amino acid variants of a particular species of H-NOX domain or may comprise H-NOX domains from different species. In some embodiments, at least one of the H-NOX domains of a polymeric H-NOX protein comprises a mutation corresponding to an L144F mutation of T. tengcongensis H-NOX. In some embodiments, the polymeric H-NOX proteins comprise one or more polymerization domains. In some embodiments, the polymeric H-NOX protein is a trimeric H-NOX protein. In some embodiments, the polymeric H-NOX protein comprises at least one trimerization domain. In some embodiments, the trimeric H-NOX protein comprises three T. tengcongensis H-NOX domains. In some embodiments the trimeric H-NOX domain comprises three T. tengcongensis L144F H-NOX domains.

In some aspects of the invention, the polymeric H-NOX protein comprises two or more associated monomers. The monomers may be covalently linked or noncovalently linked. In some embodiments, monomeric subunits of a polymeric H-NOX protein are produced where the monomeric subunits associate in vitro or in vivo to form the polymeric H-NOX protein. In some embodiments, the monomers comprise an H-NOX domain and a polymerization domain. In some embodiments, the polymerization domain is covalently linked to the H-NOX domain; for example, the C-terminus of the H-NOX domain is covalently linked to the N-terminus or the C-terminus of the polymerization domain. In other embodiments, the N-terminus of the H-NOX domain is covalently linked to the N-terminus or the C-terminus of the polymerization domain. In some embodiments, an amino acid spacer is covalently linked between the H-NOX domain and the polymerization domain. An “amino acid spacer” and an “amino acid linker” are used interchangeably herein. In some embodiments, at least one of the monomeric subunits of a polymeric H-NOX protein comprises a mutation corresponding to an L144F mutation of T. tengcongensis H-NOX. In some embodiments the polymeric H-NOX protein is a trimeric H-NOX protein. In some embodiments, the monomer of a trimeric H-NOX protein comprises an H-NOX domain and a foldon domain of T4 bacteriophage. In some embodiments, the monomer of a trimeric H-NOX protein comprises a T. tengcongensis H-NOX domain and a foldon domain. In some embodiments, the monomer of a trimeric H-NOX protein comprises a T. tengcongensis L144F H-NOX domain and a foldon domain. In some embodiments, the trimer H-NOX protein comprises three monomers, each monomer comprising a T. tengcongensis L144F H-NOX domain and a foldon domain. In some embodiments, the H-NOX domain is linked to the foldon domain with an amino acid linker; for example a Gly-Ser-Gly linker. In some embodiments, at least one H-NOX domain comprises a tag. In some embodiments, at least one H-NOX domain comprises a His₆ tag. In some embodiments, the His₆ tag is linked to the foldon domain with an amino acid linker; for example an Arg-Gly-Ser linker. In some embodiments, all of the H-NOX domains comprise a Hiss tag. In some embodiments, the trimeric H-NOX protein comprises the amino acid sequence set forth in SEQ ID NO:6 or SEQ ID NO:8.

The exemplary H-NOX domain from T. tengcongensis is approximately 26.7 kDal. In some embodiments, the polymeric H-NOX protein has an atomic mass greater than any of about 50 kDal, 75 kDal, 100 kDal, 125 kDal, to about 150 kDal.

The invention provides polymeric H-NOX proteins that show greater accumulation in one or more tissues in an individual compared to a corresponding monomeric H-NOX protein comprising a single H-NOX domain following administration of the H-NOX protein to the individual. A corresponding H-NOX protein refers to a monomeric form of the H-NOX protein comprising at least one of the H-NOX domains of the polymeric 1-NOX protein. Tissues of preferential polymeric 1-NOX accumulation include, but are not limited to tissue with damaged vasculature. In some embodiments the polymeric H-NOX protein persists in a mammal for at least about 1, 2, 3, 4, 6, 12 or 24 hours or at least about 1, 2, 3, 4, 5, 6, 7, 8, 9 or 10 days following administration of the H-NOX protein to the individual. In some embodiments the polymeric H-NOX protein persists in a mammal for about 1-2, 2-3, 3-4, 4-6, 6-12 or 12-24 hours or 1-2 days, 2-4 days, 4-8 days, 8-10 days or greater than 10 days following administration of the H-NOX protein to the individual. In some embodiments, less than about 10% of the polymeric H-NOX is cleared from mammal by the kidneys within less than any of about 1 hour, 2 hours or 3 hours following administration of the H-NOX protein to the individual.

### 7.2.3 Sources of H-NOX Proteins and H-NOX Domains

H-NOX proteins and H-NOX domains from any genus or species can be used in the compositions, kits, and methods described herein. In various embodiments, the H-NOX protein or the H-NOX domains of a polymeric H-NOX protein is a protein or domain from a mammal (e.g., a primate (e.g., human, monkey, gorilla, ape, lemur, etc), a bovine, an equine, a porcine, a canine, or a feline), an insect, a yeast, or a bacteria or is derived from such a protein Exemplary mammalian H-NOX proteins include wild-type human and rat soluble guanylate cyclase (such as the β1 subunit). Examples of H-NOX proteins include wild-type mammalian H-NOX proteins, e.g. H. sapiens, M. musculus, C. familiaris, B. Taurus, C. lupus and R. norvegicus: and wild-type non-mammalian vertebrate H-NOX proteins, e.g., X. laevis, O. latipes, O. curivatus, and F. rubripes. Examples of non-mammalian wild-type NO-binding H-NOX proteins include wild-type H-NOX proteins of D. melanogaster, A. gambiae, and M. sexta; examples of non-mammalian wild-type O₂-binding H-NOX proteins include wild-type H-NOX proteins of C. elegans gcy-31, gcy-32, gcy-33, gcy-34, gcy-35, gcy-36, and gcy-37, D. melanogaster CG14885, CG14886, and CG4154; and M. sexta beta-3; examples of prokaryotic wild-type H-NOX proteins include T. tengcongensis, V. cholera, V. fischerii, N. punctiforme, D. desulfuricans, L. pneumophila 1, L. pneumophila 2, and C. acetobutylicum.

NCBI Accession numbers for exemplary H-NOX proteins include the following: Homo sapiens β1 [gi:2746083], Rattus norvegicus β1 [gi:27127318], Drosophila melangaster β1 [gi:861203], Drosophila melangaster CG14885-PA [gi:23171476]. Caenorhabditis elegans GCY-35 [gi:52782806], Nostoc punctiforme [gi:23129606], Caulobacter crescentus [gi:16127222], Shewanella oneidensis [gi:24373702], Legionella pneumophila (ORF 2) [CUCGC_272624], Clostridium acetobutylicum [gi:15896488], and Thermoanaerobacter tengcongensis [gi:20807169]. Canis lupus H-NOX is provided by GenBank accession DQ008576. Nucleic acid and amino acid sequences of exemplary H-NOX proteins and domains are provided in Section 9 below.

Exemplary H-NOX protein also include the following H-NOX proteins that are listed by their gene name, followed by their species abbreviation and Genbank Identifiers (such as the following protein sequences available as of May 21, 2006; May 22, 2006; May 21, 2007; or May 22, 2007, which are each hereby incorporated by reference in their entireties): Npun5905_Npu_23129606, alr2278_Ana_17229770, SO2144_Sone_24373702, Mdeg1343_Mde_23027521, VCA0720_Vch_15601476, CC2992_Ccr_16127222, Rsph2043_Rhsp_22958463 (gi:46192757), Mmc10739 Mcsp_22999020, Tar4_Tte_20807169, Ddes2822_Dde_23475919, CAC3243_Cac_15896488, gcy-31_Ce_17568389, CG14885_Dm_24647455, GUCY1B3_Hs_4504215, HpGCS-beta1_Hpul_14245738, Gycbeta100B_Dm_24651577, CG4154_Dm_24646993 (gi:NP_650424.2, gi-62484298), gcy-32_Ce_13539160, gcy-36_Ce_17568391 (gi:32566352, gi:86564713), gcy-35_Ce-17507861 (gi:71990146), gcy-37_Ce_17540904 (gi:71985505), GCY1a3_Hs_20535603, GCY1a2-Hs_899477, or GYCa-99B_Dm_729270 (gi:68067738) (Lakshminarayan et al. (2003) RMG Genomics 4:5-13). The species abbreviations used in these names include Ana—Anabaena Sp; Ccr—Caulobacter crescentus; Cac—Clostridium acetobutylicum; Dde—Desulfovibrio desulfuricans; Mcsp—Magnetococcus sp.; Mde-Microbulbifer degradans; Npu—Nostoc punctiforme; Rhsp—Rhodobacter sphaeroides; Sone—Shewanella oneidensis; Tte—Thermoanaerobacter tengcongensis; Vch—Vibrio cholerae; Ce—Caenorhabditis elegans; Dm—Drosophila melanogaster; Hpul—Hemicentrotus pulcherrimus; Hs—Homo sapiens.

Other exemplary H-NOX proteins include the following H-NOX proteins that are listed by their organism name and Pfam database accession number (such as the following protein sequences available as of May 21, 2006, May 22, 2006, May 17, 2007; May 21, 2007; or May 22, 2007, which are each hereby incorporated by reference in their entireties): Caenorhabditis briggsae Q622M5_CAEBR, Caenorhabditis briggsae Q61P44_CAEBR, Caenorhabditis briggsae Q61R54_CAEBR, Caenorhabditis briggsae Q61V90_CAEBR, Caenorhabditis briggsae Q61A94_CAEBR, Caenorhabditis briggsae Q60TP4_CAEBR, Caenorhabditis briggsae Q60M10_CAEBR, Caenorhabditis elegans GCY37_CAEEL, Caenorhabditis elegans GCY31_CAEEL, Caenorhabditis elegans GCY36_CAEEL, Caenorhabditis elegans GCY32_CAEEL, Caenorhabditis elegans GCY35_CAEEL, Caenorhabditis elegans GCY34_CAEEL, Caenorhabditis elegans GCY33_CAEEL, Oryzias curvinotus Q7T040_ORYCU, Oryzias curvinotus Q75WF0_ORYCU, Oryzias latipes P79998_ORYLA, Oryzias latipes Q7ZSZ5_ORYLA, Tetraodon nigroviridis Q4SW38_TETNG, Tetraodon nigroviridis Q4RZ94_TETNG, Tetraodon nigroviridis Q4S6K5_TETNG, Fugu rubripes Q90VY5_FUGRU, Xenopus laevis Q61NK9_XENLA, Homo sapiens Q5T8J7_HUMAN, Homo sapiens GCYA2_HUMAN, Homo sapiens GCYB2_HUMAN, Homo sapiens GCYB1_HUMAN, Gorilla gorilla Q9N193_9PRIM, Pongo pygmaeus Q5RAN8_PONPY, Pan troglodytes Q9N192_PANTR, Macaca mulatta Q9N194_MACMU. Hylobates lar Q9N191_HYLLA, Mus musculus Q8BXH3_MOUSE, Mus musculus GCYB1_MOUSE, Mus musculus Q3UTI4_MOUSE, Mus musculus Q3UH83_MOUSE, Mus musculus Q6XE41_MOUSE, Mus musculus Q80YP4_MOUSE, Rattus norvegicus Q80WX7 RAT, Rattus norvegicus Q80WX8 RAT, Rattus norvegicus Q920Q1_RAT, Rattus norvegicus Q54A43_RAT, Rattus norvegicus Q80WY0_RAT, Rattus norvegicus Q80WY4_RAT, Rattus norvegicus Q8CH85_RAT, Rattus norvegicus Q80WY5 RAT, Rattus norvegicus GCYB1_RAT, Rattus norvegicus Q8CH90_RAT, Rattus norvegicus Q91XJ7_RAT, Rattus norvegicus Q80WX9_RAT, Rattus norvegicus GCYB2_RAT, Rattus norvegicus GCYA2_RAT, Canis familiaris Q4ZHR9_CANFA, Bos taurus GCYB1_BOVIN, Sus scrofa Q4ZHR7_PTG, Gryllus bimaculatus Q591N5_GRYBI, Manduca sexta 077106_MANSE, Manduca sexta 076340_MANSE, Apis mellifera Q5UAF0_APIME, Apis mellifera Q5FANO_APIME, Apis mellifera Q6L5L6_APIME, Anopheles gambiae str PEST Q7PYK9_ANOGA, Anopheles gambiae str PEST Q7Q9W6_ANOGA, Anopheles gambiae str PEST Q7QF31_ANOGA, Anopheles gambiae str PEST Q7PS01_ANOGA, Anopheles gambiae str PEST Q7PFY2_ANOGA, Anopheles gambiae Q7KQ93_ANOGA, Drosophila melanogaster Q24086_DROME, Drosophila melanogaster GCYH_DROME, Drosophila melanogaster GCY8E_DROME, Drosophila melanogaster GCYDA_DROME, Drosophila melanogaster GCYDB_DROME, Drosophila melanogaster Q9VA09_DROME, Drosophila pseudoobscura Q29CE1_DROPS, Drosophila pseudoobscura Q296C7 DROPS, Drosophila pseudoobscura Q296C8 DROPS, Drosophila pseudoobscura Q29BU7_DROPS, Aplysia californica Q7YWK7_APLCA, Hemicentrotus pulcherrimus Q95NK5_HEMPU, Chlamydomonas reinhardtii, Q5YLC2_CHLRE, Anabaena sp Q8YUQ7_ANASP, Flavobacteria bacterium BBFL7 Q26GR8_9BACT, Psychroflexus torquis ATCC 700755 Q1VQE5_9FLAO, marine gamma proteobacterium HTCC2207 Q1YPJ5_9GAMM, marine gamma proteobacterium HTCC2207 Q1YTK4_9GAMM, Caulobacter crescentus Q9A451_CAUCR, Acidiphilium cryptum JF-5 Q2DG60_ACICY, Rhodobacter sphaeroides Q3JOU9_RHOS4, Silicibacter pomeroyi QSLPV1_SILPO, Paracoccus denitrificas PD1222, Q3PC67_PARDE, Silicibacter sp TM1040 Q3QNY2_9RHOB, Jannaschia sp Q28ML8_JANSC, Magnetococcus sp MC-1 Q3XT27_9PROT, Legionella pneumophila Q5WXPO_LEGPL, Legionella pneumophila Q5WTZ5_LEGPL, Legionella pneumophila Q5X268_LEGPA, Legionella pneumophila Q5X2R2_LEGPA, Legionella pneumophila subsp pneumophila Q5ZWM9_LEGPH, Legionella pneumophila subsp pneumophila Q5ZSQ8_LEGPH, Colwellia psychrerythraea Q47Y43_COLP3, Pseudoalteromonas atlantica T6c Q3CSZ5_ALTAT, Shewanella oneidensis Q8EF49_SHEON, Saccharophagus degradans Q21E20_SACD2, Saccharophagus degradans Q21ER7_SACD2, Vibrio angustum S14 Q1ZWES_9VIBR, Vibrio vulnificus Q8DAE2_VIBVU, Vibrio alginolylicus 12G01 Q1VCP6_VIBAL, Vibrio sp DAT722 Q2FA22_9VIBR, Vibrio parahaemolyticus Q87NJ1 VIBPA, Vibrio fischeri Q5E1F5_VIBF1, Vibrio vulnificus Q7MJS8_VIBVY, Photobacterium sp SKA34 Q2C6Z5_9GAMM, Hahella chejuensis Q2SFY7_HAHCH, Oceanospirillum sp MED92 Q2BKV0_9GAMM, Oceanobacter sp RED65 Q1N035_9GAMM, Desulfovibrio desulfuricans Q310U7_DESDG, Halothermothrix orenii H 168 Q2AIW5_9FIRM, Thermoanaerobacter tengcongensis Q8RBX6_THETN, Caldicellulosiruptor saccharolyticus DSM 8903 Q2ZH17_CALSA, Clostridium acetobutylicum Q97E73_CLOAB, Alkaliphilus metalliredigenes QYMF Q3C763_9CLOT, Clostridium tetani Q899J9_CLOTE, and Clostridium beijerincki NC1AMIB8052 Q2WVN0_CLOBE. These sequences are predicted to encode H-NOX proteins based on the identification of these proteins as belonging to the H-NOX protein family using the Pfam database as described herein.

Additional H-NOX proteins, H-NOX domains of polymeric H-NOX proteins, and nucleic acids, which may be suitable for use in the pharmaceutical compositions and methods described herein, can be identified using standard methods. For example, standard sequence alignment and/or structure prediction programs can be used to identify additional H-NOX proteins and nucleic acids based on the similarity of their primary and/or predicted protein secondary structure with that of known H-NOX proteins and nucleic acids. For example, the Pfam database uses defined alignment algorithms and Hidden Markov Models (such as Pfam 21.0) to categorize proteins into families, such as the H-NOX protein family (Pfam—A database of protein domain family alignments and Hidden Markov Models, Copyright (C) 1996-2006 The Pfam Consortium; GNU LGPL Free Software Foundation, Inc., 59 Temple Place—Suite 330, Boston, Mass. 02111-1307, USA). Standard databases such as the swissprot-trembl database (world-wide web at “expasy.org”, Swiss Institute of Bioinformatics Swiss-Prot group CMU—1 rue Michel Servet CH-1211 Geneva 4, Switzerland) can also be used to identify members of the H-NOX protein family. The secondary and/or tertiary structure of an H-NOX protein can be predicted using the default settings of standard structure prediction programs, such as PredictProtein (630 West, 168 Street, BB217, New York, N.Y. 10032, USA). Alternatively, the actual secondary and/or tertiary structure of an H-NOX protein can be determined using standard methods.

In some embodiments, the H-NOX domain has the same amino acid in the corresponding position as any of following distal pocket residues in T. tengcongensis H-NOX: Thr4, Ile5, Thr8, Trp9, Trp67, Asn74, Ile75, Phe78, Phe82, Tyr140, Leu144, or any combination of two or more of the foregoing. In some embodiments, the H-NOX domain has a proline or an arginine in a position corresponding to that of Pro115 or Arg135 of T. tengcongensis H-NOX, respectively, based on sequence alignment of their amino acid sequences. In some embodiments, the H-NOX domain has a histidine that corresponds to His105 of R. norvegicus β1 H-NOX. In some embodiments, the H-NOX domain has or is predicted to have a secondary structure that includes six alpha-helices, followed by two beta-strands, followed by one alpha-helix, followed by two beta-strands. This secondary structure has been reported for H-NOX proteins.

If desired, a newly identified H-NOX protein or H-NOX domain can be tested to determine whether it binds heme using standard methods. The ability of an H-NOX domain to function as an O₂ carrier can be tested by determining whether the H-NOX domain binds O₂ using standard methods, such as those described herein. If desired, one or more of the mutations described herein can be introduced into the H-NOX domain to optimize its characteristics as an O₂ carrier. For example, one or more mutations can be introduced to alter its O₂ dissociation constant, k_{off} for oxygen, rate of heme autoxidation, NO reactivity, NO stability or any combination of two or more of the foregoing. Standard techniques such as those described herein can be used to measure these parameters.

### 7.2.4 Mutant H-NOX Proteins

As discussed further herein, an H-NOX protein or an H-NOX domain of a polymeric H-NOX protein may contain one or more mutations, such as a mutation that alters the O₂ dissociation constant, the k_{off} for oxygen, the rate of heme autoxidation, the NO reactivity, the NO stability, or any combination of two or more of the foregoing compared to that of the corresponding wild-type protein. In some embodiments, the invention provides a polymeric H-NOX protein comprising one or more H-NOX domains that may contain one or more mutations, such as a mutation that alters the O₂ dissociation constant, the koff for oxygen, the rate of heme autoxidation, the NO reactivity, the NO stability, or any combination of two or more of the foregoing compared to that of the corresponding wild-type protein. Panels of engineered H-NOX domains may be generated by random mutagenesis followed by empirical screening for requisite or desired dissociation constants, dissociation rates, NO-reactivity, stability, physio-compatibility, or any combination of two or more of the foregoing in view of the teaching provided herein using techniques as described herein and, additionally, as known by the skilled artisan Alternatively, mutagenesis can be selectively targeted to particular regions or residues such as distal pocket residues apparent from the experimentally determined or predicted three-dimensional structure of an H-NOX protein (see, for example, Boon, E. M. et al. (2005) Nature Chemical Biology 1:53-59, which is hereby incorporated by reference in its entirety, particularly with respect to the sequences of wild-type and mutant H-NOX proteins) or evolutionarily conserved residues identified from sequence alignments (see, for example, Boon E. M. et al. (2005) Nature Chemical Biology 1:53-59, which is hereby incorporated by reference in its entirety, particularly with respect to the sequences of wild-type and mutant H-NOX proteins).

In some embodiments of the invention, the mutant H-NOX protein or mutant H-NOX domain of a polymeric H-NOX protein has a sequence that differs from that of all H-NOX proteins or domains occurring in nature. In various embodiments, the amino acid sequence of the mutant protein is at least about any of 10, 15, 20, 25, 30, 40, 50, 60, 70, 80, 90, 95, 97, 98, 99, or 99.5% identical to that of the corresponding region of an H-NOX protein occurring in nature. In various embodiments, the amino acid sequence of the mutant protein is about 10-20%, 20-30%, 30-40%, 40-50%, 50-60%, 60-70%, 70-80%, 80-90%, 90-95%, 95-99%, or 99.5% identical to that of the corresponding region of an H-NOX protein occurring in nature. In some embodiments, the mutant protein is a protein fragment that contains at least about any of 25, 50, 75, 100, 150, 200, 300, or 400 contiguous amino acids from a full-length protein. In some embodiments, the mutant protein is a protein fragment that contains 25-50, 50-75, 75-100, 100-150, 150-200, 200-300, or 300-400 contiguous amino acids from a full-length protein. Sequence identity can be measured, for example, using sequence analysis software with the default parameters specified therein (e.g., Sequence Analysis Software Package of the Genetics Computer Group, University of Wisconsin Biotechnology Center, 1710 University Avenue, Madison, Wis. 53705). This software program matches similar sequences by assigning degrees of homology to various amino acids replacements, deletions, and other modifications.

In some embodiments of the invention, the mutant H-NOX protein or mutant H-NOX domain of a polymeric H-NOX protein comprises the insertion of one or more amino acids (e.g., the insertion of 2, 3, 4, 5, 6, 7, 8, 9, or 10 amino acids). In some embodiments of the invention, the mutant H-NOX protein or mutant H-NOX domain comprises the deletion of one or more amino acids (e.g., a deletion of N-terminal, C-terminal, and/or internal residues, such as the deletion of at least about any of 5, 10, 15, 25, 50, 75, 100, 150, 200, 300, or more amino acids or a deletion of 5-10, 10-15, 15-25, 25-50, 50-75, 75-100, 100-150, 150-200, 200-300, or 300-400 amino acids). In some embodiments of the invention, the mutant H-NOX protein or mutant H-NOX domain comprises the replacement of one or more amino acids (e.g., the replacement of 1, 2, 3, 4, 5, 6, 7, 8, 9, or 10 amino acids), or combinations of two or more of the foregoing. In some embodiments, a mutant protein has at least one amino acid alteration compared to a protein occurring in nature. In some embodiments, a mutant nucleic acid sequence encodes a protein that has at least one amino acid alteration compared to a protein occurring in nature. In some embodiments, the nucleic acid is not a degenerate version of a nucleic acid occurring in nature that encodes a protein with an amino acid sequence identical to a protein occurring in nature.

In some embodiments the mutation in the H-NOX protein or H-NOX domain of a polymeric H-NOX protein is an evolutionary conserved mutations (also denoted class I mutations). Examples of class I mutations are listed in Table 1A. In Table 1A, mutations are numbered/annotated according to the sequence of human β1 H-NOX, but are analogous for all H-NOX sequences. Thus, the corresponding position in any other H-NOX protein can be mutated to the indicated residue. For example, Phe4 of human β1 H-NOX can be mutated to a tyrosine since other H-NOX proteins have a tyrosine in this position. The corresponding phenylalanine residue can be mutated to a tyrosine in any other H-NOX protein. In particular embodiments, the one or more mutations are confined to evolutionarily conserved residues. In some embodiments, the one or more mutations may include at least one evolutionarily conserved mutation and at least one non-evolutionarily conserved mutation. If desired, these mutant H-NOX proteins are subjected to empirical screening for NO/O₂ dissociation constants, NO-reactivity, stability, and physio-compatibility in view of the teaching provided herein.

TABLE 1A
Exemplary Class I H-NOX mutations targeting
evolutionary conserved residues

| | | |
|---|---|---|
| F4Y | Q30G | I145Y |
| F4L | E33P | I145H |
| H7G | N61G | K151E |
| A8E | C78H | I157F |
| L9W | A109F | E183F |

In some embodiments, the mutation is a distal pocket mutation, such as mutation of a residue in alpha-helix A, D, E, or G (Pellicena, P. et al. (Aug. 31, 2004) Proc Natl. Acad Sci USA 101(35):12854-12859). Exemplary distal pocket mutations (also denoted class II mutations) are listed in Table 1B. In Table 1B, mutations are numbered/annotated according to the sequence of human β1 H-NOX, but are analogous for all H-NOX sequences. Because several substitutions provide viable mutations at each recited residue, the residue at each indicated position can be changed to any other naturally or non-naturally-occurring amino acid (denoted “X”). Such mutations can produce H-NOX proteins with a variety of desired affinity, stability, and reactivity characteristics.

TABLE 1B
Exemplary Class II H-NOX mutations
targeting distal pocket residues

| | | |
|---|---|---|
| V8X | M73X | I145X |
| L9X | F77X | I149X |
| F70X | C78X | |

In particular embodiments, the mutation is a heme distal pocket mutation. As described herein, a crucial molecular determinant that prevents O₂ binding in NO-binding members of the H-NOX family is the lack of an H-bond donor in the distal pocket of the heme. Accordingly, in some embodiments, the mutation alters H-bonding between the H-NOX domain and the ligand within the distal pocket. In some embodiments, the mutation disrupts an H-bond donor of the distal pocket and/or imparts reduced O₂ ligand-binding relative to the corresponding wild-type H-NOX domain. Exemplary distal pocket residues include Thr4, Ile5, Thr8, Trp9, Trp67, Asn74, Ile75, Phe78, Phe82, Tyr140, and Leu144 of T. tengcongensis H-NOX and the corresponding residues in any other H-NOX protein. In some embodiments, the H-NOX protein or H-NOX domain of a polymeric H-NOX protein comprises one or more distal pocket mutations. In some embodiments, the H-NOX protein or H-NOX domain of a polymeric H-NOX protein comprises one, two, three, four, five, six, seven, eight, nine, ten or more than ten distal pocket mutations. In some embodiments, the distal pocket mutation corresponds to a L144F mutation of T. tengcongensis H-NOX. In some embodiments, the distal pocket mutation is a L144F mutation of T. tengcongensis H-NOX. In some embodiments, H-NOX protein or the H-NOX domain of a polymeric H-NOX protein comprises two distal pocket mutations.

Residues that are not in the distal pocket can also affect the three-dimensional structure of the heme group; this structure in turn affects the binding of O₂ and NO to iron in the heme group. Accordingly, in some embodiments, the H-NOX protein or H-NOX domain of a polymeric H-NOX protein has one or more mutations outside of the distal pocket. Examples of residues that can be mutated but are not in the distal pocket include Pro115 and Arg135 of T. tengcongensis H-NOX. In some embodiments, the mutation is in the proximal pocket which includes His105 as a residue that ligates to the heme iron.

In some embodiments when two or more mutations are present; at least one mutation is in the distal pocket, and at least one mutation is outside of the distal pocket (e.g., a mutation in the proximal pocket). In some embodiments, all the mutations are in the distal pocket.

To reduce the immunogenicity of H-NOX protein or H-NOX domains derived from sources other than humans, amino acids in an H-NOX protein or H-NOX domain can be mutated to the corresponding amino acids in a human H-NOX. For example, one or more amino acids on the surface of the tertiary structure of a non-human H-NOX protein or H-NOX domain can be mutated to the corresponding amino acid in a human H-NOX protein or H-NOX domain. In some variations, mutation of one or more surface amino acids may be combined with mutation of two or more distal pocket residues, mutation of one or more residues outside of the distal pocket (e.g., a mutation in the proximal pocket), or combinations of two or more of the foregoing.

The invention also relates to any combination of mutation described herein, such as double, triple, or higher multiple mutations. For example, combinations of any of the mutations described herein can be made in the same H-NOX protein. Note that mutations in equivalent positions in other mammalian or non-mammalian H-NOX proteins are also encompassed by this invention. Exemplary mutant H-NOX proteins or mutant H-NOX domains comprise one or more mutations that impart altered O₂ or NO ligand-binding relative to the corresponding wild-type H-NOX domain and are operative as a physiologically compatible mammalian O₂ blood gas carrier.

The residue number for a mutation indicates the position in the sequence of the particular H-NOX protein being described. For example, T. tengcongensis TSA refers to the replacement of isoleucine by alanine at the fifth position in T. tengcongensis H-NOX. The same isoleucine to alanine mutation can be made in the corresponding residue in any other H-NOX protein or H-NOX domain (this residue may or may not be the fifth residue in the sequence of other H-NOX proteins). Since the amino acid sequences of mammalian β1 H-NOX domains differ by at most two amino acids, mutations that produce desirable mutant H-NOX proteins or H-NOX domains when introduced into wild-type rat β1 H-NOX proteins are also expected to produce desirable mutant H-NOX proteins or H-NOX domains when introduced into wild-type β1 H-NOX proteins or H-NOX domains from other mammals, such as humans.

In some embodiments, the H-NOX protein is a trimer comprising three T. tengcongensis L144F H-NOX domains and three foldon domains. In some embodiments, the H-NOX protein is a trimer comprising three T. tengcongensis wildtype H-NOX domains and three foldon domains.

### 7.2.5. Modifications to H-NOX Proteins

Any of the wild-type or mutant H-NOX proteins, including polymeric H-NOX proteins, can be modified and/or formulated using standard methods to enhance therapeutic or industrial applications. For example, and particularly as applied to heterologous engineered H-NOX proteins, a variety of methods are known in the art for insulating such agents from immune surveillance, including crosslinking, PEGylation, carbohydrate decoration, etc. (e.g., Rohlfs, R. J. et al. (May 15, 1998) J. Biol. Chem. 273(20):12128-12134; Migita, R. et al. (June 1997) J. Appl. Physiol. 82(6):1995-2002; Vandegriff, K. D. et al. (Aug. 15, 2004) Biochem J. 382(Pt 1):183-189, which are each hereby incorporated by reference in their entireties, particularly with respect to the modification of proteins) as well as other techniques known to the skilled artisan. Fusing an H-NOX protein, including a polymeric H-NOX protein, with a human protein such as human serum albumin can increase the serum half-life, viscosity, and colloidal oncotic pressure. In some embodiments, an H-NOX protein is modified during or after its synthesis to decrease its immunogenicity and/or to increase its plasma retention time. H-NOX proteins can also be encapsulated (such as encapsulation within liposomes or nanoparticles).

In some embodiments, the H-NOX protein is covalently bound to polyethylene glycol (PEG). An H-NOX protein covalently bound to polyethylene glycol can be referred to as PEGylated (referred to herein as “H-NOXP”). An H-NOX protein that is not covalently bound to polyethylene glycol can be referred to as non-PEGylated. In some embodiments, the H-NOXP protein is a trimer comprising three T. tengcongensis L144F H-NOX domains and three foldon domains. In some embodiments, at least one monomer of a trimeric H-NOXP protein is PEGylated. In some embodiments, each monomer of a trimeric H-NOXP protein is PEGylated. In some embodiments, a monomeric H-NOX comprises three PEG molecules. In some embodiments, a trimeric H-NOX comprises nine PEG molecules (three for each monomer). In some embodiments the PEG has a molecular weight of 5000.

In certain embodiments, the H-NOX protein used in the compositions and methods provided herein is a polymeric H-NOX protein (e.g., a trimeric H-NOX protein) comprising one, two, three, four, five, six, or seven PEG molecules per monomer. In a preferred embodiment, the H-NOX protein used in the compositions and methods provided herein is a polymeric H-NOX protein (e.g., a trimeric H-NOX protein) comprising three PEG molecules per monomer. In certain embodiments, the PEG molecule has a molecular weight between 1 kDa and 10 kDa, or between 5 kDa and 10 kDa. In a preferred embodiment, the PEG molecule has a molecular weight of about 5 kDa (e.g., 5 kDa). In one embodiment, the PEG molecule is a linear methoxy PEG (m-PEG). In one embodiment, the H-NOX protein used in the compositions and methods provided herein is a polymeric H-NOX protein (e.g., a trimeric H-NOX protein, preferably a T. tengcongensis L144F trimeric H-NOX protein) comprising three PEG molecules per monomer, wherein each of the PEG molecule has a molecular weight of 5 kDa and, optionally, wherein each of the PEG molecules is a linear methoxy PEG (m-PEG). In one embodiment, the H-NOX protein used in the compositions and methods provided herein is a T. tengcongensis L144F trimeric H-NOX protein comprising three PEG molecules per monomer, wherein each of the PEG molecule has a molecular weight of about 5 kDa and, optionally, wherein each of the PEG molecules is a linear methoxy PEG (m-PEG). As will be understood by a person skilled in the art, the foregoing numbers of PEG molecules per monomer are average values.

In some embodiments, both PEGylated and non-PEGylated H-NOX is administered to an individual to treat any disorder or condition described herein. In some embodiments, the PEGylated and non-PEGylated H-NOX is administered at the same time. In some embodiments, the ratio of PEGylated to non-PEGylated H-NOX administered to the individual is any of about 99:1, 95:5, 90:10, 85:15, 80:20, 75:25, 70:30, 65:35, 60:40; 55:45, 50:50, 45:55, 40:60, 35:65, 30:70, 25:75, 20:80, 15:85, 10:90; 5:95; 1:99 or any ratio therebetween. In some embodiments, the PEGylated and non-PEGylated H-NOX is in a composition. In some embodiments, the PEGylated and non-PEGylated H-NOX is in the composition at a ratio of about any of 99:1, 95:5, 90:10, 85:15, 80:20, 75:25, 70:30, 65:35, 60:40; 55:45, 50:50, 45:55, 40:60, 35:65, 30:70, 25:75, 20:80, 15:85; 10:90, 5:95; 1:99 or any ratio therebetween. In some embodiments, the H-NOX proteins include a PEGylated trimeric H-NOX protein comprising three T. tengcongensis L144F H-NOX domains and three foldon domains. In some embodiments, the H-NOX proteins include a non-PEGylated trimeric H-NOX protein comprising three T. tengcongensis L144F H-NOX domains and three foldon domains. In some embodiments, the H-NOX proteins include a PEGylated trimeric H-NOX protein comprising three T. tengcongensis L144F H-NOX domains and three foldon domains and a non-PEGylated trimeric H-NOX protein comprising three T. tengcongensis L144F H-NOX domains and three foldon domains.

In some embodiments, the H-NOX protein comprises one of more tags, e.g. to assist in purification of the H-NOX protein. Examples of tags include, but are not limited to His6, FLAG, GST, and MBP. In some embodiments, the H-NOX protein comprises one of more His tags. The one or more His6 tags may be removed prior to use of the polymeric H-NOX protein; e.g. by treatment with an exopeptidase. In some embodiments, the H-NOX protein is a trimer comprising three T. tengcongensis L144F H-NOX domains, three foldon domains, and three His₆ tags. In some embodiments, the H-NOX protein is a trimer comprising three T. tengcongensis wildtype H-NOX domains, three foldon domains, and three His₆ tags.

### 7.2.6. Polymerization Domains

In some aspects, the invention provides polymeric H-NOX proteins comprising two or more H-NOX domains and one or more polymerization domains. Polymerization domains are used to link two or more H-NOX domains to form a polymeric H-NOX protein. One or more polymerization domains may be used to produce dimers, trimers, tetramers, pentamers, etc. of H-NOX proteins. Polymerization domains are known in the art, such as: the foldon of T4 bacteriophage fibritin, Arc, POZ, coiled coil domains (including GCN4, leucine zippers, Velcro), uteroglobin, collagen, 3-stranded coiled colis (matrilin-1), thrombosporins, TRPV1-C, P53, Mnt, avadin, streptavidin, Bcr-Abl, COMP, verotoxin subunit B, CamKII, RCK, and domains from N ethylmaleimide-sensitive fusion protein, STM3548, KaiC, TyrR, Hcp1, CcmK4, GP41, anthrax protective antigen, aerolysin, a-hemolysin, C4b-binding protein, Mi-CK, arylsurfatase A, and viral capsid proteins. The polymerization domains may be covalently or non-covalently linked to the H-NOX domains. In some embodiments, a polymerization domain is linked to an H-NOX domain to form a monomer subunit such that the polymerization domains from a plurality of monomer subunits associate to form a polymeric H-NOX domain. In some embodiments, the C-terminus of an H-NOX domain is linked to the N-terminus of a polymerization domain. In other embodiments, the N-terminus of an H-NOX domain is linked to the N-terminus of a polymerization domain. In yet other embodiments, the C-terminus of an H-NOX domain is linked to the C-terminus of a polymerization domain. In some embodiments, the N-terminus of an H-NOX domain is linked to the C-terminus of a polymerization domain.

Linkers may be used to join a polymerization domain to an H-NOX domain; for example, for example, amino acid linkers. In some embodiments, a linker comprising any one of one, two, three, four, five, six, seven, eight, nine, ten or more than ten amino acids may be placed between the polymerization domain and the H-NOX domain. Exemplary linkers include but are not limited to Gly-Ser-Gly and Arg-Gly-Ser linkers.

### 7.2.7. Bacteriophage T4fibritin Trimerization Domain

An exemplary polymerization domain is the foldon domain of bacteriophage T4. The wac gene from the bacteriophage T4 encodes the fibritin protein, a 486 amino acid protein with a C-terminal trimerization domain (residues 457-483) (Efimov, V. P. et al. (1994) J Mol Biol 242:470-486). The domain is able to trimerize fibritin both in vitro and in vivo (Boudko, S. P et al. (2002) Eur J Biochem 269:833-841; Letarov, A. V., et al., (1999) Biochemistry (Mosc) 64:817-823; Tao, Y., et al., (1997) Structure 5:789-798). The isolated 27 residue trimerization domain, often referred to as the “foldon domain,” has been used to construct chimeric trimers in a number of different proteins (including HIV envelope glycoproteins (Yang, X. et al., (2002) J Virol 76:4634-4642), adenoviral adhesins (Papanikolopoulou. K., et al., (2004) J Biol Chem 279.8991-8998; Papanikolopoulou, K. et al. (2004) J Mol Biol 342:219-227), collagen (Zhang, C., et al. (2009) Biotechnol Prog 25:1660-1668), phage P22 gp26 (Bhardwaj, A., et al. (2008) Protein Sci 17.1475-1485), and rabies virus glycoprotein (Sissoeff, L., et al. (2005) J Gen Virol 86:2543-2552). An exemplary sequence of the foldon domain is provided by SEQ ID NO:4.

The isolated foldon domain folds into a single β-hairpin structure and trimerizes into a β-propeller structure involving three hairpins (Guthe, S. et al. (2004) J. Mol. Biol. 337:905-915). The structure of the foldon domain alone has been determined by NMR (Guthe, S. et al. (2004) J Mol. Biol. 337:905-915) and the structures of several proteins trimerized with the foldon domain have been solved by X-ray crystallography (Papanikolopoulou, K., et al., (2004), J Biol Chem 279:8991-8998; Stetefeld, J. et a (2003) Structure 11:339-346; Yokoi, N. et al (2010) Small 6:1873-1879). The domain folds and trimerizes rapidly reducing the opportunity for misfolding intermediates or off-pathway oligomerization products (Guthe, S. et al. (2004) J. Mol. Biol. 337:905-915). The foldon domain is very stable, able to maintain tertiary structure and oligomerization in >10% SDS, 6.0M guanidine hydrochloride, or 80° C. (Bhardwaj, A., et al. (2008)Protein Sci. 17:1475-1485; Bhardwaj, A., et al. (2007) J. Mol. Biol. 371:374-387) and can improve the stability of sequences fused to the foldon domain (Du, C. et al. (2008) Appl. Microbiol. Biotechnol. 79:195-202.

In some embodiments, the C-terminus of an H-NOX domain is linked to the N-terminus of a foldon domain. In other embodiments, the N-terminus of an H-NOX domain is linked to the N-terminus of a foldon domain. In yet other embodiments, the C-terminus of an H-NOX domain is linked to the C-terminus of a foldon domain. In some embodiments, the N-terminus of an H-NOX domain is linked to the C-terminus of a foldon domain.

In some embodiments, linkers are be used to join a foldon domain to an H-NOX domain. In some embodiments, a linker comprising any one of one, two, three, four, five, six, seven, eight, nine, ten or more than ten amino acids may be placed between the polymerization domain and the H-NOX domain. Exemplary linkers include but are not limited to Gly-Ser-Gly and Arg-Gly-Ser linkers. In some embodiments, the invention provides a trimeric H-NOX protein comprising from N-terminus to C-terminus: a T. tengcongensis H-NOX domain, a Gly-Ser-Gly amino acid linker, and a foldon domain. In some embodiments, the invention provides a trimeric H-NOX protein comprising from N-terminus to C-terminus: a T. tengcongensis H-NOX domain, a Gly-Ser-Gly amino acid linker, a foldon domain, an Arg-Gly-Ser amino acid linker, and a His₆ tag. In some embodiments, the T. tengcongensis H-NOX domain comprises an L144F mutation. In some embodiments, the T. tengcongensis H-NOX domain is a wild-type H-NOX domain.

### 7.2.8 Monomeric H-NOX Domain Subunits

In one aspect, the invention provides recombinant monomeric H-NOX proteins (i.e. monomeric H-NOX subunits of polymeric H-NOX proteins) that can associate to form polymeric H-NOX proteins. In some embodiments, the invention provides recombinant H-NOX proteins comprising an H-NOX domain as described herein and a polymerization domain. The H-NOX domain and the polymerization domain may be covalently linked or noncovalently linked. In some embodiments, the C-terminus of an H-NOX domain of the recombinant monomeric H-NOX protein is linked to the N-terminus of a polymerization domain. In other embodiments, the N-terminus of an H-NOX domain of the recombinant monomeric H-NOX protein is linked to the N-terminus of a polymerization domain. In yet other embodiments, the C-terminus of an H-NOX domain of the recombinant monomeric H-NOX protein is linked to the C-terminus of a polymerization domain. In some embodiments, the N-terminus of an H-NOX domain of the recombinant monomeric H-NOX protein is linked to the C-terminus of a polymerization domain. In some embodiments, the recombinant monomeric H-NOX protein does not comprise a guanylyl cyclase domain.

In some embodiments, the monomeric H-NOX protein comprises a wild-type H-NOX domain. In some embodiments of the invention, the monomeric H-NOX protein comprises one of more mutations in the H-NOX domain. In some embodiments, the one or more mutations alter the O₂ dissociation constant, the k_{off} for oxygen, the rate of heme autooxidation, the NO reactivity, the NO stability or any combination of two or more of the foregoing compared to that of the corresponding wild-type H-NOX domain. In some embodiments, the mutation is a distal pocket mutation. In some embodiments, the mutation comprises a mutation that is not in the distal pocket. In some embodiments, the distal pocket mutation corresponds to a L144 mutation of T. tengcongensis (e.g. a L144F mutation).

In some aspects, the invention provides recombinant monomeric H-NOX proteins that associate to form trimeric H-NOX proteins. In some embodiments, the recombinant H-NOX protein comprises an H-NOX domain and a trimerization domain. In some embodiments, the trimerization domain is a foldon domain as discussed herein. In some embodiments, the H-NOX domain is a T. tengcongensis H-NOX domain. In some embodiments the C-terminus of the T. tengcongensis H-NOX domain is covalently linked to the N-terminus of the foldon domain. In some embodiments the C-terminus of the T. tengcongensis H-NOX domain is covalently linked to the C-terminus of the foldon domain. In some embodiments, the T. tengcongensis domain is an L144F H-NOX domain. In some embodiments, the T. tengcongensis domain is a wild-type H-NOX domain.

In some embodiments, the H-NOX domain is covalently linked to the polymerization domain using an amino acid linker sequence. In some embodiments, the amino acid linker sequence is one, two, three, four, five, six, seven, eight, nine, ten or more than ten amino acids in length. Exemplary amino acid linker sequences include but are not limited to a Gly-Ser-Gly sequence and an Arg-Gly-Ser sequence. In some embodiments, the polymeric 1-NOX protein is a trimeric H-NOX protein comprising three 11-NOX domains and three trimerization sequences wherein the H-NOX domain is covalently linked to the trimerization domain via an amino acid linker sequence. In some embodiments, the monomeric H-NOX protein comprises the following from the N-terminus to the C-terminus, a T. tengcongensis L144F H-NOX domain, a Gly-Ser-Gly amino acid linker sequence, and a foldon domain. In some embodiments, the monomeric H-NOX protein comprises the following from the N-terminus to the C-terminus: a wild-type T. tengcongensis H-NOX domain, a Gly-Ser-Gly amino acid linker sequence, and a foldon domain.

In some embodiments, the recombinant monomeric H-NOX protein comprises a tag; e.g., A His₆, a FLAG, a GST, or an MBP tag. In some embodiments, the recombinant monomeric H-NOX protein comprises a His₆ tag. In some embodiments, the recombinant monomeric H-NOX protein does not comprise a tag. In some embodiments, the tag (e.g. a His₆ tag) is covalently linked to the polymerization domain using an amino acid spacer sequence. In some embodiments, the amino acid linker sequence is one, two, three, four, five, six, seven, eight, nine, ten or more than ten amino acids in length. Exemplary amino acid linker sequences include but are not limited to a Gly-Ser-Gly sequence and an Arg-Gly-Ser sequence. In some embodiments, the polymeric H-NOX protein is a trimeric H-NOX protein comprising three H-NOX domains, three trimerization sequences, and three His₆ tags, wherein the H-NOX domain is covalently linked to the trimerization domain via an amino acid linker sequence and the trimerization domain is covalently linked to the His₆ tag via an amino acid linker sequence. In some embodiments, the monomeric H-NOX protein comprises the following from the N-terminus to the C-terminus: an L144F T. tengcongensis H-NOX domain, a Gly-Ser-Gly amino acid linker sequence, a foldon domain, an Arg-Gly-Ser linker sequence, and a His₆ tag. In some embodiments, the monomeric H-NOX protein comprises the following from the N-terminus to the C-terminus; a wild-type T. tengcongensis H-NOX domain, a Gly-Ser-Gly amino acid linker sequence, a foldon domain, an Arg-Gly-Ser linker sequence, and a His₆ tag.

In some embodiments the recombinant monomeric H-NOX protein comprises the amino acid sequence of SEQ ID NO:6 or SEQ ID NO:8.

### 7.2.9. Characteristics of Wild-type and Mutant H-NOX Proteins

As described herein, a large number of diverse H-NOX mutant proteins, including polymeric H-NOX proteins, providing ranges of NO and O₂ dissociation constants, O₂ k_{off}, NO reactivity, and stability have been generated. To provide operative blood gas carriers, the H-NOX proteins may be used to functionally replace or supplement endogenous O₂ carriers, such as hemoglobin. In some embodiments, H-NOX proteins such as polymeric H-NOX proteins are used to deliver O₂ to hypoxic tissue (e.g. a hypoxic penumbra). Accordingly, in some embodiments, an H-NOX protein has a similar or improved O₂ association rate, O₂ dissociation rate, dissociation constant for O₂ binding, NO stability, NO reactivity, autoxidation rate, plasma retention time, or any combination of two or more of the foregoing compared to an endogenous O₂ carrier, such as hemoglobin. In some embodiments, the H-NOX protein is a polymeric H-NOX protein. In some embodiments, the polymeric H-NOX protein is a trimeric H-NOX protein comprising three monomers, each monomer comprising a T. tengcongensis L144F H-NOX domain and a foldon domain. In some embodiments, the polymeric H-NOX protein is a trimeric H-NOX protein comprising three monomers, each monomer comprising a T. tengcongensis L144F H-NOX domain and a foldon domain.

In various embodiments, the k_{off} for O₂ for an H-NOX protein, including a polymeric H-NOX protein, is between about 0.01 to about 200 s⁻¹ at 20° C., such as about 0.1 to about 200 s⁻¹, about 0.1 to 100 s⁻¹, about 1.0 to about 16.0 s⁻¹, about 1.35 to about 23.4 s⁻¹, about 1.34 to about 18 s⁻¹, about 1.35 to about 14.5 s⁻¹, about 0.21 to about 23.4 s⁻¹, about 1.35 to about 2.9 s⁻¹, about 2 to about 3 s⁻¹, about 5 to about 15 s⁻¹, or about 0.1 to about 1 s⁻¹. In some embodiments, the H-NOX protein has a k_{off} for oxygen that is less than or equal to about 0.65 s⁻¹ at 20° C. (such as between about 0.21 s⁻¹ to about 0.65 s⁻¹ at 20° C.).

In various embodiments, the kₒₙ for O₂ for an H-NOX protein, including a polymeric H-NOX protein, is between about 0.14 to about 60 μM⁻¹s⁻¹ at 20° C., such as about 6 to about 60 μM⁻¹s⁻¹, about 6 to 12 μM⁻¹s⁻¹, about 15 to about 60 μM⁻¹s⁻¹, about 5 to about 18 μM⁻¹s⁻¹, or about 6 to about 15 μM⁻¹s⁻¹.

In various embodiments, the kinetic or calculated K_{D} for O₂ binding by an H-NOX protein, including a polymeric H-NOX protein, is between about 1 nM to 1 mM, about 1 μM to about 10 μM, or about 10 μM to about 50 μM. In some embodiments the calculated K_{D} for Oz binding is any one of about 2 nM to about 2 μM, about 2p M to about 1 mM, about 100 nM to about 1 μM, about 9 μM to about 50 μM, about 100 μM to about 1 mM, about 50 nM to about 10 μM, about 2 nM to about 50 μM, about 100 nM to about 1.9 μM, about 150 nM to about 1 μM, or about 100 nM to about 255 nM, about 20 nM to about 2 μM, 20 nM to about 75 nM, about 1 μM to about 2 μM, about 2 μM to about 10 μM, about 2 μM to about 9 μM, or about 100 nM to 500 nM at 20° C. In some embodiments, the kinetic or calculated K_{D} for Oz binding is less than about any of 100 nM, 80 nM, 50 nM, 30 nM, 25 nM, 20 nM, or 10 nM at 20° C.

In various embodiments, the kinetic or calculated K_{D} for O₂ binding by an H-NOX protein, including a polymeric H-NOX protein, is within about 0.01 to about 100-fold of that of hemoglobin under the same conditions (such as at 20° C.), such as between about 0.1 to about 10-fold or between about 0.5 to about 2-fold of that of hemoglobin under the same conditions (such as at 20° C.). In various embodiments, the kinetic or calculated K_{D} for NO binding by an H-NOX protein is within about 0.01 to about 100-fold of that of hemoglobin under the same conditions (such as at 20° C.), such as between about 0.1 to about 10-fold or between about 0.5 to about 2-fold of that of hemoglobin under the same conditions (such as at 20° C.).

In some embodiments, less than about any of 50, 40, 30, 10, or 5% of an H-NOX protein, including a polymeric H-NOX protein, is oxidized after incubation for about any of 1, 2, 4, 6, 8, 10, 15, or 20 hours at 20° C.

In various embodiments, the NO reactivity of an H-NOX protein, including a polymeric H-NOX protein, is less than about 700 s⁻¹ at 20° C., such as less than about 600 s⁻¹, 500 s⁻¹, 400 s⁻¹, 300 s⁻¹, 200 s⁻¹, 100 s⁻¹, 75 s⁻¹, 50 s⁻¹, 25 s⁻¹, 20 s⁻¹, 10 s⁻¹, 50 s⁻¹, 3 s⁻¹, 2 s⁻¹, 1.8 s⁻¹, 1.5 s⁻¹, 1.2 s⁻¹, 1.0 s⁻¹, 0.8 s⁻¹, 0.7 s⁻¹, or 0.6 s⁻¹ at 20° C. In various embodiments, the NO reactivity of an H-NOX protein is between about 0.1 to about 600 s⁻¹ at 20° C., such as between about 0.5 to about 400 s⁻¹, about 0.5 to about 100 s⁻¹, about 0.5 to about 50 s⁻¹, about 0.5 to about 10 s⁻¹, about 1 to about 5 s⁴, or about 0.5 to about 2.1 s⁻¹ at 20° C. In various embodiments, the reactivity of an H-NOX protein is at least about 10, 100, 1,000, or 10,000 fold lower than that of hemoglobin under the same conditions, such as at 20° C.

In various embodiments, the rate of heme autoxidation of an H-NOX protein, including a polymeric H-NOX protein, is less than about 1.0 h⁻¹ at 37° C., such as less than about any of 0.9 h⁻¹, 0.8 h⁻¹, 0.7 h⁻¹, 0.6 h⁻¹, 0.5 h⁻¹, 0.4 h⁻¹, 0.3 h⁻¹, 0.2 h⁻¹, 0.1 h⁻¹, or 0.05 h⁻¹ at 37 C. In various embodiments, the rate of heme autoxidation of an H-NOX protein is between about 0.006 to about 5.0 h⁻¹ at 37° C., such as about 0.006 to about 1.0 h⁻¹, 0.006 to about 0.9 h⁻¹, or about 0.06 to about 0.5 h⁻¹ at 37° C.

In various embodiments, a mutant H-NOX protein, including a polymeric H-NOX protein, has (a) an O₂ or NO dissociation constant, association rate (kon for O₂ or NO), or dissociation rate (k_{off} for O₂ or NO) within 2 orders of magnitude of that of hemoglobin, (b) has an NO affinity weaker (e.g., at least about 10-fold, 100-fold, or 1000-fold weaker) than that of sGC β1, respectively, (c) an NO reactivity with bound O₂ at least 1000-fold less than hemoglobin, (d) an in vivo plasma retention time at least 2, 10, 100, or 1000-fold higher than that of hemoglobin, or (e) any combination of two or more of the foregoing.

Exemplary suitable O₂ carriers provide dissociation constants within two orders of magnitude of that of hemoglobin, i.e. between about 0.01 and 100-fold, such as between about 0.1 and 10-fold, or between about 0.5 and 2-fold of that of hemoglobin A variety of established techniques may be used to quantify dissociation constants, such as the techniques described herein (Boon, E. M. et al. (2005) Nature Chem. Biol. 1:53-59; Boon. E. M. et al. (October 2005) Curr. Opin. Chem. Biol. 9(5):441-446; Boon, E. M. et al. (2005). J. Inorg. Biochem. 99(4):892-902), Vandegriff, K. D. et al. (Aug. 15, 2004) Biochem J. 382(Pt 1):183-189, which are each hereby incorporated by reference in their entireties, particularly with respect to the measurement of dissociation constants), as well as those known to the skilled artisan. Exemplary O₂ carriers provide low or minimized NO reactivity of the H-NOX protein with bound O₂, such as an NO reactivity lower than that of hemoglobin. In some embodiments, the NO reactivity is much lower, such as at least about 10, 100, 1,000, or 10,000-fold lower than that of hemoglobin. A variety of established techniques may be used to quantify NO reactivity (Boon, E. M. et al. (2005) Nature Chem. Biol. 1:53-59: Boon, E. M. et al. (October 2005) Curr. Opin. Chem. Biol. 9(5):441-446; Boon, E. M. et al. (2005). J. Inorg. Biochem. 99(4):892-902), Vandegriff, K. D. et al. (Aug. 15, 2004) Biochem J. 382(Pt 1):183-189, which are each hereby incorporated by reference in their entireties, particularly with respect to the measurement of NO reactivity) as well as those known to the skilled artisan. Because wild-type T. tengcongensis H-NOX has such a low NO reactivity, other wild-type H-NOX proteins and mutant H-NOX proteins may have a similar low NO reactivity. For example, T. tengcongensis H-NOX Y140H has an NO reactivity similar to that of wild-type T. tengcongensis H-NOX.

In addition, suitable O₂ carriers provide high or maximized stability, particularly in vivo stability A variety of stability metrics may be used, such as oxidative stability (e.g., stability to autoxidation or oxidation by NO), temperature stability, and in vivo stability. A variety of established techniques may be used to quantify stability, such as the techniques described herein (Boon, E. M. et al. (2005) Naure Chem. Biol. 1.53-59, Boon, E. M. et al. (October 2005) Curr. Opin. Chem. Biol. 9(5):441-446; Boon, E. M. et al. (2005)J Inorg. Biochem. 99(4):892-902), as well as those known to the skilled artisan. For in vivo stability in plasma, blood, or tissue, exemplary metrics of stability include retention time, rate of clearance, and half-life. H-NOX proteins from thermophilic organisms are expected to be stable at high temperatures. In various embodiments, the plasma retention times are at least about 2-, 10-, 100-, or 1000-fold greater than that of hemoglobin (e.g. Bobofchak, K. M. et al. (August 2003) Am. J Physiol. Heart Circ. Physiol. 285(2):H549-H561). As will be appreciated by the skilled artisan, hemoglobin-based blood substitutes are limited by the rapid clearance of cell-free hemoglobin from plasma due the presence of receptors for hemoglobin that remove cell-free hemoglobin from plasma. Since there are no receptors for H-NOX proteins in plasma, wild-type and mutant H-NOX proteins are expected to have a longer plasma retention time than that of hemoglobin. If desired, the plasma retention time can be increased by PEGylating or crosslinking an H-NOX protein or fusing an H-NOX protein with another protein using standard methods (such as those described herein and those known to the skilled artisan).

In various embodiments, the H-NOX protein, including a polymeric H-NOX protein, has an O₂ dissociation constant between about 1 nM to about 1 mM at 20° C. and a NO reactivity at least about 10-fold lower than that of hemoglobin under the same conditions, such as at 20° C. In some embodiments, the H-NOX protein has an O₂ dissociation constant between about 1 nM to about 1 mM at 20° C. and a NO reactivity less than about 700 s⁻¹ at 20° C. (e.g., less than about 600 s⁻¹, 500 s⁻¹, 100 s⁻¹, 20 s⁻¹, or 1.8 s⁻¹ at 20° C.). In some embodiments, the H-NOX protein has an O₂ dissociation constant within 2 orders of magnitude of that of hemoglobin and a NO reactivity at least about 10-fold lower than that of hemoglobin under the same conditions, such as at 20° C. In some embodiments, the H-NOX protein has a k_{off} for oxygen between about 0.01 to about 200 s⁻¹ at 20° C. and an NO reactivity at least about 10-fold lower than that of hemoglobin under the same conditions, such as at 20° C. In some embodiments, the H-NOX protein has a k_{off} for oxygen that is less than about 0.65 s⁻¹ at 20° C. (such as between about 0.21 s⁻¹ to about 0.64 s⁻¹ at 20° C.) and a NO reactivity at least about 10-fold lower than that of hemoglobin under the same conditions, such as at 20° C. In some embodiments of the invention, the O₂ dissociation constant of the H-NOX protein is between about 1 nM to about 1 μM (1000 nM), about 1 μM to about 10 μM, or about 10 μM to about 50 μM. In particular embodiments, the O₂ dissociation constant of the H-NOX protein is between about 2 nM to about 50 μM, about 50 nM to about 10 μM, about 100 nM to about 1.9 μM, about 150 nM to about 1 μM, or about 100 nM to about 255 nM at 20° C. In various embodiments, the O₂ dissociation constant of the H-NOX protein is less than about 80 nM at 20° C., such as between about 20 nM to about 75 nM at 20° C. In some embodiments, the NO reactivity of the H-NOX protein is at least about 100-fold lower or about 1,000 fold lower than that of hemoglobin, under the same conditions, such as at 20° C. In some embodiments, the NO reactivity of the H-NOX protein is less than about 700 s⁻¹ at 20° C., such as less than about 600 s⁻¹, 500 s⁻¹, 400 s⁻¹, 300 s⁻¹, 200 s⁻¹, 100 s⁻¹, 75 s⁻¹, 50 s⁻¹, 25 s⁻¹, 20 s⁻¹, 10 s⁻¹, 50 s⁻¹, 3 s⁻¹, 2 s⁻¹, 1.8 s⁻¹, 1.5 s⁻¹′, 1.2 s⁻¹, 1.0 s⁻¹, 0.8 s⁻¹, 0.7 s⁻¹, or 0.6 s⁻¹ at 20° C. In some embodiments, the k_{off} for oxygen of the H-NOX protein is between 0.01 to 200 s⁻¹ at 20° C., such as about 0.1 to about 200 s⁻¹, about 0.1 to 100 s⁻¹, about 1.35 to about 23.4 s⁻¹, about 1.34 to about 18 s⁻¹, about 1.35 to about 14.5 s⁻¹, about 0.21 to about 23.4 s⁻¹, about 2 to about 3 s⁻¹, about 5 to about 15 s⁻¹, or about 0.1 to about 1 s⁻¹. In some embodiments, the O₂ dissociation constant of the H-NOX protein is between about 100 nM to about 1.9 μM at 20° C., and the k_{off} for oxygen of the H-NOX protein is between about 1.35 s⁻¹ to about 14.5 s⁻¹ at 20° C. In some embodiments, the rate of heme autoxidation of the H-NOX protein is less than about 1 h⁻¹ at 37° C., such as less than about any of 0.9 h⁻¹, 0.8 h⁻¹, 0.7 h⁻¹, 0.6 h⁻¹, 0.5 h⁻¹, 0.4 h⁻¹, 0.3 h⁻¹, 0.2 h⁻¹, or 0.1 h⁻¹. In some embodiments, the k_{off} for oxygen of the H-NOX protein is between about 1.35 s⁻¹ to about 14.5 s⁻¹ at 20° C., and the rate of heme autoxidation of the H-NOX protein is less than about 1 h⁻¹ at 37° C. In some embodiments, the koff for oxygen of the H-NOX protein is between about 1.35 s⁻¹ to about 14.5 s⁻¹ at 20° C., and the NO reactivity of the H-NOX protein is less than about 700 s⁻¹ at 20° C. (e.g., less than about 600 s⁻¹, 500 s⁻¹, 100 s⁻¹, 20 s⁻¹, or 1.8 s⁻¹ at 20° C.). In some embodiments, the rate of heme autoxidation of the H-NOX protein is less than about 1 h⁻¹ at 37° C. and the NO reactivity of the H-NOX protein is less than about 700 s⁻¹ at 20° C. (e.g., less than about 600 s⁻¹, 500 s⁻¹, 100 s⁻¹, 20 s⁻¹, or 1.8 s⁻¹ at 20° C.).

In some embodiments, the viscosity of the H-NOX protein solution, including a polymeric H-NOX protein solution, is between 1 and 4 centipoise (cP). In some embodiments, the colloid oncotic pressure of the H-NOX protein solution is between 20 and 50 mm Hg.

7.2.10. Measurement of O₂ and/or NO Binding

One skilled in the art can readily determine the oxygen and nitric oxide binding characteristics of any H-NOX protein including a polymeric H-NOX protein such as a trimeric H-NOX protein by methods known in the art and by the non-limiting exemplary methods described below.

7.2.11. Kinetic K_{D}: Ratio of k_{off} to kₒₙ

The kinetic K_{D} value is determined for wild-type and mutant H-NOX proteins, including polymeric H-NOS proteins, essentially as described by Boon, E. M. et al. (2005). Nature Chemical Biology 1:53-59, which is hereby incorporated by reference in its entirety, particularly with respect to the measurement of O₂ association rates, O₂ dissociation rates, dissociation constants for O₂ binding, autoxidation rates, and NO dissociation rates.

7.2.11.1. kₒₙ (O₂ Association Rate)

O₂ association to the heme is measured using flash photolysis at 20° C. It is not possible to flash off the Fe^{II}-O₂ complex as a result of the very fast geminate recombination kinetics; thus, the Fe^{II}-CO complex is subjected to flash photolysis with laser light at 560 nm (Hewlett-Packard, Palo Alto, Calif.), producing the 5-coordinate Fe^{II} intermediate, to which the binding of molecular O₂ is followed at various wavelengths. Protein samples are made by anaerobic reduction with 10 mM dithionite, followed by desalting on a PD-10 column (Millipore, Inc., Billerica, Mass.). The samples are then diluted to 20 μM heme in 50 mM TEA, 50 mM NaCl, pH 7.5 buffer in a controlled-atmosphere quartz cuvette, with a size of 100 μL to 1 mL and a path-length of 1 cm. CO gas is flowed over the headspace of this cuvette for 10 minutes to form the Fe^{II}-CO complex, the formation of which is verified by UV-visible spectroscopy (Soret maximum 423 nm). This sample is then either used to measure CO-rebinding kinetics after flash photolysis while still under 1 atmosphere of CO gas, or it is opened and stirred in air for 30 minutes to fully oxygenate the buffer before flash photolysis to watch O₂-rebinding events. O₂ association to the heme is monitored at multiple wavelengths versus time. These traces are fit with a single exponential using Igor Pro software (Wavemetrics, Inc., Oswego, Oreg.; latest 2005 version). This rate is independent of observation wavelength but dependent on O₂ concentration. UV-visible spectroscopy is used throughout to confirm all the complexes and intermediates (Cary 3K, Varian, Inc. Palo Alto, Calif.). Transient absorption data are collected using instruments described in Dmochowski, I. J. et al. (Aug. 31, 2000) J. Inorg. Biochem. 81(3):221-228, which is hereby incorporated by reference in its entirety, particularly with respect to instrumentation. The instrument has a response time of 20 ns, and the data are digitized at 200 megasamples s⁻¹.

7.2.11.2. k_{off} (O₂Dissociation Rate)

To measure the k_{off}, Fe^{II}-O₂ complexes of protein (5 μM heme), are diluted in anaerobic 50 mM TEA, 50 mM NaCl, pH 7.5 buffer, and are rapidly mixed with an equal volume of the same buffer (anaerobic) containing various concentrations of dithionite and/or saturating CO gas. Data are acquired on a HI-TECH Scientific SF-61 stopped-flow spectrophotometer equipped with a Neslab RTE-100 constant-temperature bath set to 20° C. (TGK Scientific LTD., Bradford On Avon, United Kingdom). The dissociation of O₂ from the heme is monitored as an increase in the absorbance at 437 nm, a maximum in the Fe^{II}-Fe^{II}-O₂ difference spectrum, or 425 nm, a maximum in the Fe^{II}-Fe^{II}-CO difference spectrum. The final traces are fit to a single exponential using the software that is part of the instrument. Each experiment is done a minimum of six times, and the resulting rates are averaged. The dissociation rates measured are independent of dithionite concentration and independent of saturating CO as a trap for the reduced species, both with and without 10 mM dithionite present.

### 7.2.11.3. Kinetic K_{D}

The kinetic K_{D} is determined by calculating the ratio of koff to kon using the measurements of k_{off} and kₒₙ described above.

### 7.2.11.3.1. Calculated K_{D}

To measure the calculated K_{D}, the values for the k_{off} and kinetic K_{D} that are obtained as described above are graphed. A linear relationship between k_{off} and kinetic K_{D} is defined by the equation (y=mx+b). koff values were then interpolated along the line to derive the calculated K_{D} using Excel: MAC 2004 (Microsoft, Redmond, Wash.). In the absence of a measured kon, this interpolation provides a way to relate k_{off} to K_{D}.

### 7.2.12. Rate of Autoxidation

To measure the rate of autoxidation, the protein samples are anaerobically reduced, then diluted to 5 μM heme in aerobic 50 mM TEA, 50 mM NaCl, pH 7.5 buffer. These samples are then incubated in a Cary 3E spectrophotometer equipped with a Neslab RTE-100 constant-temperature bath set to 37° C. and scanned periodically (Cary 3E, Varian, Inc., Palo Alto, Calif.). The rate of autoxidation is determined from the difference between the maximum and minimum in the Fe^{III}-Fe^{II} difference spectrum plotted versus time and fit with a single exponential using Excel. MAC 2004 (Microsoft, Redmond, Wash.).

7.2.13. Rate of Reaction with NO

NO reactivity is measured using purified proteins (H-NOX, polymeric H-NOX, Homo sapiens hemoglobin (Hs Hb) etc.) prepared at 2 μM in buffer A and NO prepared at 200 μM in Buffer A (Buffer A: 50 mM Hepes, pH 7.5, 50 mM NaCl). Data are acquired on a HI-TECH Scientific SF-61 stopped-flow spectrophotometer equipped with a Neslab RTE-100 constant-temperature bath set to 20° C. (TGK Scientific LTD., Bradford On Avon, United Kingdom). The protein is rapidly mixed with NO in a 1:1 ratio with an integration time of 0.00125 sec. The wavelengths of maximum change are fit to a single exponential using the software that is part of the spectrometer, essentially measuring the rate-limiting step of oxidation by NO. The end products of the reaction are ferric-NO for the HNOX proteins and ferric-aquo for Hs Hb.

7.2.14. p50 measurements

If desired, the p50 value for mutant or wild-type H-NOX proteins can be measured as described by Guarnone, R. et al. (September/October 1995) Haematologica 80(5):426-430, which is hereby incorporated by reference in its entirety, particularly with respect to the measurement of p50 values. The p50 value is determined using a HemOx analyzer. The measurement chamber starts at 0% a oxygen and slowly is raised, incrementally, towards 100% oxygen. An oxygen probe in the chamber measures the oxygen saturation %. A second probe (UV-Vis light) measures two wavelengths of absorption, tuned to the alpha and beta peaks of the hemoprotein's (e.g., a protein such as H-NOX complexed with heme) UV-Vis spectra. These absorption peaks increase linearly as hemoprotein binds oxygen. The percent change from unbound to 100% bound is then plotted against the % oxygen values to generate a curve. The p50 is the point on the curve where 50% of the hemoprotein is bound to oxygen.

Specifically, the Hemox-Analyzer (TCS Scientific Corporation, New Hope, Pa.) determines the oxyhemoprotein dissociation curve (ODC) by exposing 50 μL of blood or hemoprotein to an increasing partial pressure of oxygen and deoxygenating it with nitrogen gas. A Clark oxygen electrode detects the change in oxygen tension, which is recorded on the x-axis of an x-y recorder. The resulting increase in oxyhemoprotein fraction is simultaneously monitored by dual-wavelength spectrophotometry at 560 nm and 576 nm and displayed on the y-axis. Blood samples are taken from the antemedial vein, anticoagulated with heparin, and kept at 4° C. on wet ice until the assay Fifty μL of whole blood are diluted in 5 μL of Hemox-solution, a manufacturer-provided buffer that keeps the pH of the solution at a value of 7.4±0.01. The sample-buffer is drawn into a cuvette that is part of the Hemox-Analyzer and the temperature of the mixture is equilibrated and brought to 37° C.; the sample is then oxygenated to 100% with air. After adjustment of the pO₂ value the sample is deoxygenated with nitrogen; during the deoxygenation process the curve is recorded on graph paper. The P50 value is extrapolated on the x-axis as the point at which O₂ saturation is 50% using the software that is part of the Hemox-Analyzer. The time required for a complete recording is approximately 30 minutes.

### 7.3. H-NOX Nucleic Acids

The invention also features nucleic acids encoding any of the mutant H-NOX proteins, polymeric H-NOX, or recombinant monomer H-NOX protein subunits as described herein, and which can be used to recombinantly express these molecules.

In particular embodiments, the nucleic acid includes a segment of or the entire nucleic acid sequence of any of nucleic acids encoding an H-NOX protein or an H-NOX domain. In some embodiments, the nucleic acid includes at least about 50, 100, 150, 200, 300, 400, 500, 600, 700, 800, or more contiguous nucleotides from a H-NOX nucleic acid and contains one or more mutations (e.g., 1, 2, 3, 4, 5, 6, 7, 8, 9, or 10 mutations) compared to the H-NOX nucleic acid from which it was derived. In various embodiments, a mutant H-NOX nucleic acid contains less than about 20, 15, 12, 10, 9, 8, 7, 6, 5, 4, 3, or 2 mutations compared to the H-NOX nucleic acid from which it was derived. The invention also features degenerate variants of any nucleic acid encoding a mutant H-NOX protein.

In some embodiments, the nucleic acid includes nucleic acids encoding two or more H-NOX domains. In some embodiments, the nucleic acids including two or more H-NOX domains are linked such that a polymeric H-NOX protein is expressed from the nucleic acid. In further embodiments, the nucleic acid includes nucleic acids encoding one or more polymerization domains. In some embodiments, the nucleic acids including the two or more H-NOX domains and the one or more polymerization domains are linked such that a polymeric H-NOX protein is expressed from the nucleic acid.

In some embodiments, the nucleic acid includes a segment or the entire nucleic acid sequence of any nucleic acid encoding a polymerization domain. In some embodiments the nucleic acid comprises a nucleic acid encoding an H-NOX domain and a polymerization domain. In some embodiments, the nucleic acid encoding an H-NOX domain and the nucleic acid encoding a polymerization domain a linked such that the produced polypeptide is a fusion protein comprising an H-NOX domain and a polymerization domain.

In some embodiments, the nucleic acid comprises nucleic acid encoding one or more His₆ tags. In some embodiments the nucleic acid further comprised nucleic acids encoding linker sequences positioned between nucleic acids encoding the H-NOX domain, the polymerization domain and/or a His₆ tag.

In some embodiments, the invention provides a nucleic acid encoding an H-NOX domain and a foldon domain. In some embodiments, the H-NOX domain is a T. thermoanaerobacter H-NOX domain. In some embodiments, the H-NOX domain is a wild-type T. thermoanaerobacter H-NOX domain. In some embodiments, the H-NOX domain is a T. thermoanaerobacter L144F H-NOX domain.

In some embodiments, the invention provides nucleic acids encoding the following 5′ to 3′: a L144F T. tengcongensis H-NOX domain, a Gly-Ser-Gly amino acid linker sequence, and a foldon domain. In some embodiments, the invention provides nucleic acids encoding the following 5′ to 3′: a wild-type T. tengcongensis H-NOX domain, a Gly-Ser-Gly amino acid linker sequence, and a foldon domain

In some embodiments, the invention provides nucleic acids encoding the following 5′ to 3′: a L144F T. tengcongensis H-NOX domain, a Gly-Ser-Gly amino acid linker sequence, a foldon domain, an Arg-Gly-Ser linker sequence, and a His₆ tag. In some embodiments, the invention provides nucleic acids encoding the following 5′ to 3′: a wild-type T. tengcongensis H-NOX domain, a Gly-Ser-Gly amino acid linker sequence, a foldon domain, an Arg-Gly-Ser linker sequence, and a His₆ tag.

In some embodiments, the nucleic acid comprises the nucleic acid sequence set forth in SEQ ID NO:1, SEQ ID NO:5, or SEQ ID NO:7.

The invention also includes a cell or population of cells containing at least one nucleic acid encoding a mutant H-NOX protein described herein. Exemplary cells include insect, plant, yeast, bacterial, and mammalian cells. These cells are useful for the production of mutant H-NOX proteins using standard methods, such as those described herein.

In some embodiments, the invention provides a cell comprising a nucleic acid encoding an H-NOX domain and a foldon domain. In some embodiments, the H-NOX domain is a T. thermoanaerobacter H-NOX domain. In some embodiments, the H-NOX domain is a wild-type T. thermoanaerobacter H-NOX domain. In some embodiments, the H-NOX domain is a T. thermoanaerobacter L144F H-NOX domain. In some embodiments, the invention provides a cell comprising a nucleic acid comprising the nucleic acid sequence set forth in SEQ ID NO:1, SEQ ID NO:5, or SEQ ID NO:7.

### 7.4. Formulations of H-NOX Proteins

Any wild-type or mutant H-NOX protein, including polymeric H-NOX proteins, described herein may be used for the formulation of pharmaceutical or non-pharmaceutical compositions. In some embodiments, the formulations comprise a monomeric H-NOX protein comprising an H-NOX domain and a polymerization domain such that the monomeric H-NOX proteins associate in vitro or in vivo to produce a polymeric H-NOX protein. As discussed further below, these formulations are useful in a variety of therapeutic and industrial applications.

In some embodiments, the pharmaceutical composition includes one or more wild-type or mutant H-NOX proteins described herein including polymeric H-NOX proteins and a pharmaceutically acceptable carrier or excipient. Examples of pharmaceutically acceptable carriers or excipients include, but are not limited to, any of the standard pharmaceutical carriers or excipients such as phosphate buffered saline solutions, water, emulsions such as oil/water emulsion, and various types of wetting agents. Exemplary diluents for aerosol or parenteral administration are phosphate buffered saline or normal (0.9%) saline. Compositions comprising such carriers are formulated by well-known conventional methods (see, for example, Remington's Pharmaceutical Sciences, 18th edition, A. Gennaro, ed., Mack Publishing Co., Easton, Pa., 1990; and Remington, The Science and Practice of Pharmacy 20th Ed. Mack Publishing, 2000, which are each hereby incorporated by reference in their entireties, particularly with respect to formulations) In some embodiments, the formulations are sterile. In some embodiments, the formulations are essentially free of endotoxin.

While any suitable carrier known to those of ordinary skill in the art may be employed in the pharmaceutical compositions of this invention, the type of carrier will vary depending on the mode of administration. Compositions can be formulated for any appropriate manner of administration, including, for example, intravenous, intra-arterial, intravesicular, inhalation, intraperitoneal, intrapulmonary, intramuscular, subcutaneous, intra-tracheal, transmucosal, intraocular, intrathecal, intracranial, administration to CSF or transdermal administration. In some embodiments, delivery may be directly to a site of vascular occlusion or directly to hypoxic tissue. For parenteral administration, such as subcutaneous injection, the carrier may include, e.g., water, saline, alcohol, a fat, a wax, or a buffer. For oral administration, any of the above carriers or a solid carrier, such as mannitol, lactose, starch, magnesium stearate, sodium saccharine, talcum, cellulose, glucose, sucrose, or magnesium carbonate, may be employed. Biodegradable microspheres (e.g., polylactate polyglycolate) may also be used as carriers.

In some embodiments, the pharmaceutical or non-pharmaceutical compositions include a buffer (e.g., neutral buffered saline, phosphate buffered saline, etc), a carbohydrate (e.g., glucose, mannose, sucrose, dextran, etc.), an antioxidant, a chelating agent (e.g., EDTA, glutathione, etc.), a preservative, another compound useful for binding and/or transporting oxygen, an inactive ingredient (e.g., a stabilizer, filler, etc.), or combinations of two or more of the foregoing. In some embodiments, the composition is formulated as a lyophilizate. H-NOX proteins may also be encapsulated within liposomes or nanoparticles using well known technology Other exemplary formulations that can be used for H-NOX proteins are described by, e.g., U.S. Pat. Nos. 6,974,795, and 6,432,918, which are each hereby incorporated by reference in their entireties, particularly with respect to formulations of proteins.

The compositions described herein may be administered as part of a sustained release formulation (e.g., a formulation such as a capsule or sponge that produces a slow release of compound following administration). Such formulations may generally be prepared using well known technology and administered by, for example, oral, rectal or subcutaneous implantation, or by implantation at the desired target site. Sustained-release formulations may contain an H-NOX protein dispersed in a carrier matrix and/or contained within a reservoir surrounded by a rate controlling membrane. Carriers for use within such formulations are biocompatible, and may also be biodegradable. In some embodiments, the formulation provides a relatively constant level of H-NOX protein release. The amount of H-NOX protein contained within a sustained release formulation depends upon the site of implantation, the rate and expected duration of release, and the nature of the condition to be treated or prevented.

In some embodiments, the pharmaceutical composition contains an effective amount of a wild-type or mutant H-NOX protein. In some embodiments, the pharmaceutical composition contains an effective amount of a polymeric H-NOX protein comprising two or more wild-type or mutant H-NOX domains. In some embodiments, the pharmaceutical composition contains an effective amount of a recombinant monomeric H-NOX protein comprising a wild-type or mutant H-NOX domain and a polymerization domain as described herein. In some embodiments, the formulation comprises a trimeric H-NOX protein comprising three monomers, each monomer comprising a T. tengcongensis L144F H-NOX domain and a foldon domain. In some embodiments, the formulation comprises a trimeric H-NOX protein comprising three monomers, each monomer comprising a T. tengcongensis L144F H-NOX domain and a foldon domain.

An exemplary dose of hemoglobin as a blood substitute is from about 10 mg to about 5 grams or more of extracellular hemoglobin per kilogram of patient body weight. Thus, in some embodiments, an effective amount of an H-NOX protein for administration to a human is between a few grams to over about 350 grams. Other exemplary doses of an H-NOX protein include about any of 4.4, 5, 10, or 13 g/dL (where g/dL is the concentration of the H-NOX protein solution prior to infusion into the circulation) at an appropriate infusion rate, such as about 0.5 ml/min (see, for example, Winslow, R. Chapter 12 In Blood Substitutes). It will be appreciated that the unit content of active ingredients contained in an individual dose of each dosage form need not in itself constitute an effective amount since the necessary effective amount could be reached by the combined effect of a plurality of administrations. The selection of the amount of an H-NOX protein to include in a pharmaceutical composition depends upon the dosage form utilized, the condition being treated, and the particular purpose to be achieved according to the determination of the ordinarily skilled artisan in the field.

Exemplary compositions include genetically engineered, recombinant H-NOX proteins, which may be isolated or purified, comprising one or more mutations that collectively impart altered O₂ or NO ligand-binding relative to the corresponding wild-type H-NOX protein, and operative as a physiologically compatible mammalian blood gas carrier. For example, mutant H-NOX proteins as described herein. In some embodiments, the H-NOX protein is a polymeric H-NOX protein. In some embodiments, the H-NOX protein is a recombinant monomeric H-NOX protein comprising a wild-type or mutant H-NOX domain and a polymerization domain as described herein. In some embodiments, the composition comprises a trimeric H-NOX protein comprising three monomers, each monomer comprising a T. tengcongensis L144F H-NOX domain and a foldon domain. In some embodiments, the composition comprises a trimeric H-NOX protein comprising three monomers, each monomer comprising a T. tengcongensis L144F H-NOX domain and a foldon domain.

To reduce or prevent an immune response in human subjects who are administered a pharmaceutical composition, human H-NOX proteins or domains (either wild-type human proteins or human proteins into which one or more mutations have been introduced) or other non-antigenic H-NOX proteins or domains (e.g., mammalian H-NOX proteins) can be used. To reduce or eliminate the immunogenicity of H-NOX proteins derived from sources other than humans, amino acids in an H-NOX protein or H-NOX domain can be mutated to the corresponding amino acids in a human H-NOX. For example, one or more amino acids on the surface of the tertiary structure of a non-human H-NOX protein can be mutated to the corresponding amino acid in a human H-NOX protein

In some embodiments, formulations of H-NOX comprise both PEGylated and non-PEGylated H-NOX. In some embodiments, the ratio of PEGylated to non-PEGylated H-NOX in the formulation is any of about 99:1, 95:5, 90:10, 85:15, 80:20, 75:25, 70:30, 65:35, 60:40; 55:45, 50:50, 45:55, 40:60, 35:65, 30:70, 25:75, 20:80, 15:85; 10:90; 5:95; 1:99 or any ratio there between. In some embodiments, formulations of H-NOX comprise both PEGylated and non-PEGylated trimeric T. tengcongensis L144F H-NOX. In some embodiments, the ratio of PEGylated to non-PEGylated trimeric T. tengcongensis L144F H-NOX in the formulation is any of about 99:1, 95:5, 90:10, 85:15, 80:20, 75:25, 70:30, 65:35, 60:40; 55:45, 50:50, 45:55, 40:60, 35:65, 30:70, 25:75, 20:80, 15:85; 10:90, 5:95; 1:99 or any ratio there between.

In certain embodiments, the H-NOX protein used in the compositions and methods described herein is a mixture comprising (i) an H-NOX protein covalently bound to polyethylene glycol (PEG), and (ii) an H-NOX protein not bound to PEG. In certain embodiments, administering the H-NOX protein comprises administering a mixture comprising (i) an H-NOX protein covalently bound to polyethylene glycol (PEG), and (ii) an H-NOX protein not bound to PEG. In certain embodiments, the mixture has a weight ratio of the H-NOX protein covalently bound to PEG to the H-NOX protein not bound to PEG of about 9:1, about 8:2, about 7:3, about 6:4, about 1:1, about 4:6, about 3:7, about 2:8, or about 1:9. In certain embodiments, the mixture has a weight ratio of the H-NOX protein covalently bound to PEG to the H-NOX protein not bound to PEG of about 99:1, 95:5, 90:10, 85:15, 80:20, 75:25, 70:30, 65:35, 60:40; 55:45, 50:50, 45:55, 40:60, 35:65, 30:70, 25:75, 20:80, 15:85; 10:90; 5:95; 1:99 or any ratio there between. In one embodiment, the weight ratio of the H-NOX protein covalently bound to PEG to the H-NOX protein not bound to PEG is about 1:1. In certain embodiments, the mixture has a weight ratio of trimeric T. tengcongensis L144F H-NOX protein covalently bound to PEG to trimeric T. tengcongensis L144F H-NOX protein not bound to PEG of about 9:1, about 8:2, about 7:3, about 6:4, about 1:1, about 4:6, about 3:7, about 2:8, or about 1:9. In certain embodiments, the mixture has a weight ratio of trimeric T. tengcongensis L144F H-NOX protein covalently bound to PEG to trimeric T. tengcongensis L144F H-NOX protein not bound to PEG of about 99:1, 95:5, 90:10, 85:15, 80:20, 75:25, 70:30, 65:35, 60:40; 55:45, 50:50, 45:55, 40:60, 35:65, 30:70, 25:75, 20:80, 15:85; 10:90; 5:95, 1:99 or any ratio there between. In one embodiment, the weight ratio of trimeric T. tengcongensis L144F H-NOX protein covalently bound to PEG to trimeric T. tengcongensis L144F H-NOX protein not bound to PEG is about 1:1.

7.4.1 H-NOX Proteins in Combination with Catecholamines

In a specific embodiment, provided herein are pharmaceutical compositions comprising (i) an H-NOX protein or a mixture of H-NOX proteins (such as any H-NOX protein or a mixture of H-NOX proteins described herein), and (ii) a catecholamine. In one embodiment, provided herein is an infusion bag comprising a composition comprising (i) an H-NOX protein or a mixture of H-NOX proteins (such as any H-NOX protein or a mixture of H-NOX proteins described herein), and (ii) a catecholamine. In certain embodiment, provided herein are methods for treating any disorder or condition described herein by administering to a subject in need thereof a pharmaceutical composition comprising (i) an H-NOX protein or a mixture of H-NOX proteins (such as any H-NOX protein or a mixture of H-NOX proteins described herein), and (ii) a catecholamine.

In another specific embodiment, an H-NOX protein or a mixture of H-NOX proteins (such as any H-NOX protein or a mixture of H-NOX proteins described herein), and a catecholamine, are administered in combination (such as concurrently or sequentially) but not in the same composition. In certain embodiments, provided herein are methods for treating any disorder or condition described herein by administering to a subject in need thereof an H-NOX protein or a mixture of H-NOX proteins (such as any H-NOX protein or a mixture of H-NOX proteins described herein) and a catecholamine.

In embodiments in which an H-NOX protein is used in combination with a catecholamine, any catecholamine described herein or known in the art may be used. In certain embodiments, the catecholamine used in the compositions and methods described herein is epinephrine, norepinephrine, dopamine, dobutamine, or atropine. In one embodiment, the catecholamine is epinephrine or norepinephrine. In one embodiment, the catecholamine is epinephrine. In one embodiment, the catecholamine is norepinephrine. In one embodiment, the catecholamine is dopamine. In one embodiment, the catecholamine is dobutamine. In one embodiment, the catecholamine is atropine.

7.5. Therapeutic Applications of H-NOX Proteins and H-NOX Proteins in Combination with Catecholamines

Any of the wild-type or mutant H-NOX proteins, including polymeric H-NOX proteins, or pharmaceutical compositions described herein may be used in therapeutic applications. Particular H-NOX proteins, including polymeric H-NOX proteins, can be selected for such applications based on the desired O₂ association rate, O₂ dissociation rate, dissociation constant for O₂ binding, NO stability, NO reactivity, autoxidation rate, plasma retention time, or any combination of two or more of the foregoing for the particular indication being treated.

Because the distribution in the vasculature of extracellular H-NOX proteins is not limited by the size of the red blood cells, polymeric H-NOX proteins of the present invention can be used to deliver O₂ to areas that red blood cells cannot penetrate. These areas can include any tissue areas that are located downstream of obstructions to red blood cell flow, such as areas downstream of one or more thrombi, arterial occlusions, peripheral vascular occlusions, angioplasty balloons, surgical instruments, tissues that are suffering from oxygen starvation or are hypoxic, and the like. Additionally, various types of tissue hypoxia or ischemia can be treated using H-NOX proteins. Such tissue ischemias include, for example, myocardial hypoxia or ischemia.

Exemplary target disorders or conditions to be treated or prevented using the compositions described here (including any H-NOX proteins or a mixture of H-NOX proteins described herein, and optionally, a catecholamine) include, without limitation, a cardiovascular disorder or condition (e.g., an impaired cardiovascular function, decreased myocardial function, myocardial hypoxia, myocardial ischemia, heart attack, cardiac arrest, congestive heart failure), a pulmonary disorder (e.g., acute respiratory failure, or depressed ventilator function), catecholamine-induced hypoxemia, anaphylaxis, hemorrhagic shock, hemorrhage, and trauma. Other exemplary target indications include, without limitation, treatment of a subject undergoing cardiac arrest, respiratory arrest or cardiopulmonary resuscitation.

In some aspects, the invention provides methods for treatment of a cardiovascular or pulmonary disorder or condition in an individual. A cardiovascular disorder or condition can be, without limitation, an impaired cardiovascular function, decreased myocardial function, myocardial hypoxia, myocardial ischemia, heart attack, cardiac arrest or congestive heart failure. A pulmonary disorder or condition can be, without limitation, an acute respiratory failure or depressed ventilator function. In one embodiment, the invention provides methods for treatment of an impaired cardiovascular function in an individual. In one embodiment, the invention provides methods for treatment of a decreased myocardial function in an individual. In one embodiment, the invention provides methods for treatment of myocardial hypoxia or myocardial ischemia in an individual. In one embodiment, the invention provides methods for treatment of a heart attack in an individual. In one embodiment, the invention provides methods for treatment of a cardiac arrest in an individual. In one embodiment, the invention provides methods for treatment of a congestive heart failure in an individual. In one embodiment, the invention provides methods for treatment of an acute respiratory failure in an individual. In one embodiment, the invention provides methods for treatment of a depressed ventilator function in an individual. In some aspects, the invention provides methods for treatment of a cardiovascular or pulmonary disorder or condition in an individual by administering an H-NOX protein (or a mixture of H-NOX proteins) to the individual. In some aspects, the invention provides methods for treatment of a cardiovascular or pulmonary disorder or condition in an individual by administering an H-NOX protein (or a mixture of H-NOX proteins) in combination with a catecholamine (e.g, epinephrine or norepinephrine) to the individual. In some embodiments, the invention provides method to deliver oxygen to an individual following an onset of a cardiovascular or pulmonary disorder or condition by administering an H-NOX protein (or a mixture of H-NOX proteins) to the individual. In some embodiments, the invention provides method to deliver oxygen to an individual following an onset of a cardiovascular or pulmonary disorder or condition by administering an H-NOX protein (or a mixture of H-NOX proteins) in combination with a catecholamine (e.g., epinephrine or norepinephrine) to the individual. In some embodiments, the H-NOX comprises H-NOX covalently bound to polyethylene glycol (PEGylated) and H-NOX that is not bound (e.g., not covalently bound) to polyethylene glycol (non-PEGylated). In some embodiments, the weight ratio of PEGylated H-NOX to non-PEGylated H-NOX administered to the individual is any of about 99:1, 95:5, 90:10, 85:15, 80:20, 75:25, 70:30, 65:35, 60:40; 55:45, 50:50, 45:55, 40:60, 35:65, 30:70, 25:75, 20:80, 15:85; 10:90; 5:95; 1:99 or any ratio therebetween. In some embodiments, the PEGylated and non-PEGylated H-NOX is in a composition. In some embodiments, the H-NOX comprises PEGylated T. tengcongensis L144F trimeric H-NOX and non-PEGylated T. tengcongensis L144F trimeric H-NOX wherein the weight ratio of PEGylated to non-PEGylated H-NOX is any of about 99:1, 95:5, 90:10, 85:15, 80:20, 75.25, 70:30, 65:35, 60:40; 55:45, 50-50, 45:55, 40:60, 35:65, 30:70, 25:75, 20:80, 15:85; 10-90; 5:95; 1-99 or any ratio therebetween.

In some aspects, the invention provides methods for treatment or prevention of a catecholamine-induced hypoxemia in an individual by administering an H-NOX protein (or a mixture of H-NOX proteins) to the individual. In some aspects, the invention provides methods for treatment or prevention of a catecholamine-induced hypoxemia in an individual by administering an H-NOX protein (or a mixture of H-NOX proteins) in combination with a catecholamine (e.g., epinephrine or norepinephrine) to the individual. In some embodiments, the invention provides methods to deliver oxygen to an individual following a catecholamine-induced hypoxemia by administering an H-NOX protein (or a mixture of H-NOX proteins) to the individual. In some embodiments, the invention provides methods to deliver oxygen to an individual following a catecholamine-induced hypoxemia by administering an H-NOX protein (or a mixture of H-NOX proteins) in combination with a catecholamine (e.g., epinephrine or norepinephrine) to the individual. In some embodiments, the H-NOX comprises H-NOX covalently bound to polyethylene glycol (PEGylated) and H-NOX that is not bound (e.g., not covalently bound) to polyethylene glycol (non-PEGylated). In some embodiments, the weight ratio of PEGylated H-NOX to non-PEGylated H-NOX administered to the individual is any of about 99:1, 95:5, 90:10, 85:15, 80:20, 75:25, 70:30, 65:35, 60.40; 55:45, 50.50, 45:55, 40:60, 35.65, 30:70, 25:75, 20.80, 15.85; 10:90; 5:95; 1.99 or any ratio therebetween. In some embodiments, the PEGylated and non-PEGylated H-NOX is in a composition.

In some aspects, the invention provides methods for treatment of anaphylaxis or hemorrhagic shock in an individual by administering an H-NOX protein (or a mixture of H-NOX proteins) to the individual. In some aspects, the invention provides methods for treatment of anaphylaxis or hemorrhagic shock in an individual by administering an H-NOX protein (or a mixture of H-NOX proteins) in combination with a catecholamine (e.g., epinephrine or norepinephrine) to the individual. In a specific aspect, the invention provides methods for treatment of anaphylaxis in an individual by administering an H-NOX protein (or a mixture of H-NOX proteins), optionally in combination with a catecholamine (e.g., epinephrine or norepinephrine), to the individual. In a specific aspect, the invention provides methods for treatment of hemorrhagic shock in an individual by administering an H-NOX protein (or a mixture of H-NOX proteins), optionally in combination with a catecholamine (e.g., epinephrine or norepinephrine), to the individual. In some embodiments, the invention provides methods to deliver oxygen to an individual following anaphylaxis or hemorrhagic shock by administering an H-NOX protein (or a mixture of H-NOX proteins) to the individual. In some embodiments, the invention provides methods to deliver oxygen to an individual following anaphylaxis or hemorrhagic shock by administering an H-NOX protein (or a mixture of H-NOX proteins) in combination with a catecholamine (e.g., epinephrine or norepinephrine) to the individual. In some embodiments, the H-NOX comprises H-NOX covalently bound to polyethylene glycol (PEGylated) and H-NOX that is not bound (e.g., not covalently bound) to polyethylene glycol (non-PEGylated). In some embodiments, the weight ratio of PEGylated H-NOX to non-PEGylated H-NOX administered to the individual is any of about 99:1, 95.5, 90:10, 85:15, 80.20, 75:25, 70:30, 65.35, 60:40; 55:45, 50:50, 45:55, 40:60, 35.65, 30:70, 25:75, 20.80, 15.85; 10:90; 5:95; 1.99 or any ratio therebetween. In some embodiments, the PEGylated and non-PEGylated H-NOX is in a composition.

In some aspects, the invention provides methods for treatment of a subject undergoing cardiac arrest, respiratory arrest or cardiopulmonary resuscitation by administering an H-NOX protein (or a mixture of H-NOX proteins) to the subject. In some aspects, the invention provides methods for treatment of a subject undergoing cardiac arrest, respiratory arrest or cardiopulmonary resuscitation by administering an H-NOX protein (or a mixture of H-NOX proteins) in combination with a catecholamine (e.g, epinephrine or norepinephrine) to the subject. In a specific aspect, the invention provides methods for treatment of a subject undergoing cardiac arrest by administering an H-NOX protein (or a mixture of H-NOX proteins), optionally in combination with a catecholamine (e.g., epinephrine or norepinephrine), to the subject. In a specific aspect, the invention provides methods for treatment of a subject undergoing respiratory arrest by administering an H-NOX protein (or a mixture of H-NOX proteins), optionally in combination with a catecholamine (e.g., epinephrine or norepinephrine), to the subject. In a specific aspect, the invention provides methods for treatment of a subject undergoing cardiopulmonary resuscitation by administering an H-NOX protein (or a mixture of H-NOX proteins), optionally in combination with a catecholamine (e.g., epinephrine or norepinephrine), to the subject. In some embodiments, the invention provides methods to deliver oxygen to an individual following a cardiac arrest or respiratory arrest by administering an H-NOX protein (or a mixture of H-NOX proteins) to the individual. In some embodiments, the invention provides methods to deliver oxygen to an individual following a cardiac arrest or respiratory arrest by administering an H-NOX protein (or a mixture of H-NOX proteins) in combination with a catecholamine (e.g., epinephrine or norepinephrine) to the individual. In some embodiments, the invention provides methods to deliver oxygen to an individual following or during cardiopulmonary resuscitation by administering an H-NOX protein (or a mixture of H-NOX proteins) to the individual. In some embodiments, the invention provides methods to deliver oxygen to an individual following or during cardiopulmonary resuscitation by administering an H-NOX protein (or a mixture of H-NOX proteins) in combination with a catecholamine (e.g., epinephrine or norepinephrine) to the individual. In some embodiments, the H-NOX comprises H-NOX covalently bound to polyethylene glycol (PEGylated) and H-NOX that is not bound (e.g., not covalently bound) to polyethylene glycol (non-PEGylated). In some embodiments, the weight ratio of PEGylated H-NOX to non-PEGylated H-NOX administered to the individual is any of about 99:1, 95:5, 90:10, 85:15, 80:20, 75:25, 70:30, 65:35, 60:40, 55:45, 50:50, 45:55, 40:60, 35:65, 30:70, 25:75, 20:80, 15:85; 10:90; 5:95; 1:99 or any ratio therebetween. In some embodiments, the PEGylated and non-PEGylated H-NOX is in a composition.

In some embodiments, the PEGylated H-NOX and the non-PEGylated H-NOX are delivered simultaneously or sequentially to treat any disorder or condition described herein in an individual. In some embodiments, the PEGylated H-NOX is administered before the non-PEGylated H-NOX. In some embodiments, the PEGylated H-NOX is delivered after the non-PEGylated H-NOX In some embodiments, the PEGylated H-NOX is delivered any of about 5 minutes, 10 minutes, 15 minutes, 30 minutes, 45 minutes, about 1 hour, about 2 hours, about 4 hours, about 6 hours, about 8 hours, about 10 hours, about 12 hours, about 16 hours, about 24 hours, about 2 days, about 3 days, about 4 days, about 5 days, about 6 days, about 7 days, about two weeks, about 3 weeks, about 4 weeks or more that about 1 month after administration of the non-PEGylated H-NOX.

In some embodiments, the PEGylated H-NOX and/or the non-PEGylate H-NOX is administered to the individual multiple times. In some embodiments, the PEGylated H-NOX and/or the non-PEGylate H-NOX is administered any of two times, three times, four times, five times, six times, seven times, ten times or more than ten times. In some embodiments, the H-NOX is administered multiple times until hypoxia or ischemia has been alleviated, or one or more symptoms of any disorder or condition described herein has been alleviated. In some embodiments, non-PEGylated H-NOX is administered to an individual suffering from any disorder or condition described herein followed by multiple administrations of PEGylated H-NOX. In some embodiments, PEGylated H-NOX is administered one or more of one hour, one day, two days, three days, four days, five days, six days, seven days, eight days, nine days or ten days after administration of non-PEGylated H-NOX.

In some embodiments, the therapeutically effective amount of an H-NOX protein is administered to the individual in conjunction with another therapy. In some embodiments, the therapeutically effective amount of PEGylated H-NOX and/or non-PEGylated H-NOX is administered to the individual in conjunction with another therapy. In some embodiments, the therapeutically effective amount of an H-NOX protein is administered to the individual in conjunction with a catecholamine (e.g., epinephrine or norepinephrine). In some embodiments, the therapeutically effective amount of PEGylated H-NOX and/or non-PEGylated H-NOX is administered to the individual in conjunction with a catecholamine (e.g., epinephrine or norepinephrine). In some embodiments, the H-NOX protein is administered in combination with mechanical or chemical recanalization of an occluded vessel. Examples of mechanical recanalization include but are not limited to angioplasty such as balloon angioplasty. Examples of chemical recanalization include but are not limited to tissue plasminogen activator (tPA). In some embodiments, the H-NOX protein is administered in combination with anti-coagulants such as heparin or warfarin (Coumadin). In some embodiments, the H-NOX is administered in combination with a neuroprotectant. In some embodiments, the H-NOX is administered before, at the same time, or after treatment with the other therapy.

In some aspects, the invention provides methods for treatment of any disorder or condition described herein in an individual comprising administering a bolus of an H-NOX protein to the individual followed by infusion of H-NOX to the individual. In some aspects, the invention provides methods for treatment of any disorder or condition described herein in an individual comprising administering a bolus of an H-NOX protein to the individual followed by infusion of H-NOX to the individual. In some aspects, the invention provides methods for treatment of any disorder or condition described herein in an individual comprising administering a bolus of an H-NOX protein to the individual by subcutaneous injection followed by infusion of H-NOX to the individual. In some aspects, the invention provides methods for treatment of any disorder or condition described herein in an individual comprising administering a bolus of an H-NOX protein to the individual by subcutaneous injection followed by infusion of H-NOX to the individual, wherein the H-NOX includes PEGylated H-NOX and/or non-PEGylated H-NOX. For example, the bolus of H-NOX may be administered in the field followed by an infusion of H-NOX in the clinic. In some embodiments, non-PEGylated H-NOX is delivered as a bolus followed by infusion of PEGylated H-NOX. In some embodiments, the H-NOX protein is administered to the individual by infusion over more than about any of 1 hour, 2 hours, 3 hours, 4 hours, 5 hours, 6 hours, 7 hours, 8 hours, 9 hours, 10 hours, 11 hours, 12 hours, 16 hours, 18 hours, 1 day, 2 days, 3 days, 4 days, 5 days, 6 days, or 7 days. In some embodiments, the H-NOX protein is administered to the individual as a bolus followed by infusion over more than about any of 1 hour, 2 hours, 3 hours, 4 hours, 5 hours, 6 hours, 7 hours, 8 hours, 9 hours, 10 hours, 11 hours, 12 hours, 16 hours, 18 hours, 1 day, 2 days, 3 days, 4 days, 5 days, 6 days, or 7 days. In some embodiments, the H-NOX protein is administered by infusion immediately after or more than about any of 1 hour, 2 hours, 3 hours, 4 hours, 5 hours, 6 hours, 7 hours, 8 hours, 9 hours, 10 hours, 11 hours, 12 hours, 16 hours, 18 hours, 1 day, 2 days, 3 days, 4 days, 5 days, 6 days, or 7 days following administration of the H-NOX protein by bolus.

In some embodiments, the H-NOX is delivered to the individual by bolus systemically followed by infusion systemically. In some embodiments, the H-NOX is delivered to the individual by bolus to the tissue affected by a disorder or condition being treated (e.g., the site of hypoxia or ischemia) followed by infusion systemically. In some embodiments, the H-NOX is delivered to the individual by bolus to the tissue affected by a disorder or condition being treated (e.g., the site of hypoxia or ischemia) followed by infusion to the affected tissue (e.g., the site of hypoxia or ischemia). In some embodiments, the H-NOX is delivered to the individual by bolus systemically followed by infusion to the tissue affected by a disorder or condition being treated (e.g., the site of hypoxia or ischemia). In some embodiments, the H-NOX is delivered by bolus and/or by infusion directly into the tissue affected by a disorder or condition being treated (e.g., the site of hypoxia or ischemia). In some embodiments, the H-NOX is delivered intramuscularly or subcutaneously.

In some embodiments, the invention provides methods for treating any disorder or condition described herein in an individual comprising administering a therapeutically effective amount of an H-NOX protein to the individual as a bolus and/or by infusion. In some embodiments, the invention provides methods for treating any disorder or condition described herein in an individual comprising administering a therapeutically effective amount of an H-NOX protein to the individual as a bolus and/or by infusion. In some embodiments, the invention provides methods for delivering O₂ to hypoxic tissue associated with any disorder or condition described herein in an individual comprising administering a therapeutically effective amount of an H-NOX protein to the individual as a bolus and/or by infusion. In some embodiments, the disorder or condition is any cardiovascular disorder or condition described herein (e.g., an impaired cardiovascular function, decreased myocardial function, myocardial hypoxia, myocardial ischemia, heart attack, cardiac arrest, congestive heart failure). In some embodiments, the disorder or condition is any pulmonary disorder or condition described herein (e.g, acute respiratory failure, or depressed ventilator function) In some embodiments, the disorder or condition is a catecholamine-induced hypoxemia, anaphylaxis, hemorrhagic shock, hemorrhage, or trauma. In some embodiments, the disorder or condition is a cardiac arrest or respiratory arrest. In some embodiments, the invention provides methods for administering a therapeutically effective amount of an H-NOX protein to the individual as a bolus and/or by infusion before, during or after cardiopulmonary resuscitation.

In some embodiments of the methods of treatment described above, the H-NOX protein of the methods of the invention is a polymeric H-NOX protein (e.g. a trimeric H-NOX protein). In some embodiments, the polymeric H-NOX protein comprises one or more H-NOX domains comprising a mutation at a position corresponding to L144 of T. tengcongensis H-NOX. In some embodiments, the polymeric H-NOX protein comprises one or more H-NOX domains comprising a mutation corresponding to a L144F mutation of T tengcongensis H-NOX. In some embodiments, the H-NOX domain is a human H-NOX domain. In some embodiments, the polymeric H-NOX protein comprises a T. tengcongensis L144F H-NOX domain.

In some aspects, the invention provides methods for treating hypoxic tissue associated with injury to an organ in an individual comprising administering a therapeutically effective amount of an H-NOX protein (or a mixture of H-NOX proteins) and a catecholamine to the individual. The hypoxia may be associated directly with the injury to the organ or may be associated indirectly with the injury. In some embodiments reducing the level of hypoxia in the tissue reduces the loss of cellular function and/or cell death which can lead to organ and/or body dysfunction. In some embodiments, the organ or tissue is part of the respiratory system or the cardiovascular system. In some embodiments, the organ is a heart or a lung.

In some aspects, the invention provides methods for delivering O₂ to hypoxic tissue associated with injury to an organ in an individual comprising administering a therapeutically effective amount of an H-NOX protein (or a mixture of H-NOX proteins) and a catecholamine to the individual. In some embodiments, the organ is a heart or a lung.

In some embodiments, the injury to the organ is a result of a vascular occlusion. For example, the injury may be due to occlusion of a coronary vessel or a vessel feeding an organ such as the lungs (e.g., a pulmonary vessel). In some embodiments, the organ injury is a result of ischemia. In some embodiments, the organ injury is a result of trauma to the organ.

In some embodiments, an H-NOX protein (or a mixture of H-NOX proteins) and a catecholamine is administered to an individual at risk of developing hypoxia associated with an injury or trauma to an organ. The H-NOX protein (or a mixture of H-NOX proteins) and a catecholamine may be administered to an individual undergoing a medical intervention in which developing hypoxia is a risk.

In various embodiments, the invention features a method of delivering O₂ to an individual (e.g., a mammal, such as a primate (e.g., a human, a monkey, a gorilla, an ape, a lemur, etc.), a bovine, an equine, a porcine, a canine, or a feline) by administering to an individual in need thereof a wild-type or mutant H-NOX protein, including a polymeric H-NOX protein in an amount sufficient to deliver O₂ to the individual. In some embodiments, the invention provides methods of carrying or delivering blood gas to an individual such as a mammal, comprising the step of delivering (e.g., transfusing, etc.) to the blood of the individual (e.g., a mammal) one or more of H-NOX compositions. Methods for delivering O₂ carriers to blood or tissues (e.g., mammalian blood or tissues) are known in the art. In various embodiments, the H-NOX protein is an apoprotein that is capable of binding heme or is a holoprotein with heme bound. The H-NOX protein may or may not have heme bound prior to the administration of the H-NOX protein to the individual. In some embodiments, O₂ is bound to the H-NOX protein before it is delivered to the individual. In other embodiments, O₂ is not bound to the H-NOX protein prior to the administration of the protein to the individual, and the H-NOX protein transports O₂ from one location in the individual to another location in the individual.

Wild-type and mutant H-NOX proteins, including polymeric H-NOX proteins, with a relatively low K_{D} for O₂ (such as less than about 80 nM or less than about 50 nM) are expected to be particularly useful to treat tissues with low oxygen tension (such as a p50 below 1 mm Hg). The high affinity of such H-NOX proteins for O₂ may increase the length of time the O₂ remains bound to the H-NOX protein, thereby reducing the amount of O₂ that is released before the H-NOX protein reaches the tissue to be treated.

In some embodiments for the direct delivery of an H-NOX protein with bound O₂ to a particular site in the body (such as a site of organ injury), the k_{off} for O₂ is more important than the K_{D} value because O₂ is already bound to the protein (making the kon less important) and oxygen needs to be released at or near a particular site in the body (at a rate influenced by the k_{off}). In some embodiments, the k_{off} may also be important when H-NOX proteins are in the presence of red cells in the circulation, where they facilitate diffusion of Oz from red cells, and perhaps prolonging the ability of diluted red cells to transport O₂ to further points in the vasculature.

In some embodiments for the delivery of an H-NOX protein that circulates in the bloodstream of an individual, the H-NOX protein binds O₂ in the lungs and releases O₂ at one or more other sites in the body. For some of these applications, the K_{D} value is more important than the k_{off} since O₂ binding is at or near equilibrium. In some embodiments for extreme hemodilution, the K_{D} is more important than the k_{off} when the H-NOX protein is the primary O₂ carrier because the H-NOX protein will bind and release O₂ continually as it travels through the circulation. Since hemoglobin has a p50 of 14 mm Hg, red cells (which act like capacitors) have a p50 of ˜30 mm Hg, and HBOCs have been developed with ranges between 5 mm Hg and 90 mm Hg, the optimal K_{D} range for H-NOX proteins may therefore be between ˜2 mm Hg to ˜100 mm Hg for some applications.

H-NOX proteins, including polymeric H-NOX proteins, and pharmaceutical compositions of the invention can be administered to an individual by any conventional means such as by oral, topical, intraocular, intrathecal, intrapulmonary, intra-tracheal, or aerosol administration; by transdermal or mucus membrane adsorption; or by injection (e.g., subcutaneous, intravenous, intra-arterial, intravesicular, or intramuscular injection). H-NOX proteins may also be included in large volume parenteral solutions for use as blood substitutes. In exemplary embodiments, the H-NOX protein is administered to the blood (e.g., administration to a blood vessel such as a vein, artery, or capillary), a wound, a hypoxic tissue, or a hypoxic organ of the individual.

In some embodiments, the H-NOX protein is delivered as a bolus. In some embodiments, the H-NOX protein is delivered by infusion. In some embodiments, the H-NOX is PEGylated. In some embodiments, the H-NOX is not PEGylated. In some embodiments, the H-NOX comprises PEGylated and non-PEGylated H-NOX. In some embodiments, the H-NOX protein is administered to the individual by infusion over more than about any of 1 hour, 2 hours, 3 hours, 4 hours, 5 hours, 6 hours, 7 hours, 8 hours, 9 hours, 10 hours, 11 hours, 12 hours, 16 hours, 18 hours, 1 day, 2 days, 3 days, 4 days, 5 days, 6 days, or 7 days. In some embodiments the H-NOX protein is administered to the individual by infusion to the injured organ or by systemic infusion. In some embodiments, the H-NOX protein is administered to the individual by a bolus followed by administration to the individual by infusion. In some embodiments, the H-NOX protein is administered to the individual as a bolus followed by infusion over more than about any of 1 hour, 2 hours, 3 hours, 4 hours, 5 hours, 6 hours, 7 hours, 8 hours, 9 hours, 10 hours, 11 hours, 12 hours, 16 hours, 18 hours, 1 day, 2 days, 3 days, 4 days, 5 days, 6 days, or 7 days. In some embodiments, the H-NOX protein is administered by infusion more than about any of 1 hour, 2 hours, 3 hours, 4 hours, 5 hours, 6 hours, 7 hours, 8 hours, 9 hours, 10 hours, 11 hours, 12 hours, 16 hours, 18 hours, 1 day, 2 days, 3 days, 4 days, 5 days, 6 days, or 7 days following administration of the H-NOX protein by bolus. In some embodiments, the H-NOX is delivered to the individual by bolus systemically followed by infusion systemically. In some embodiments, the H-NOX is delivered to the individual by bolus to the injured organ followed by infusion systemically. In some embodiments, the H-NOX is delivered to the individual by bolus to the injured organ followed by infusion to the injured organ or to the hypoxic penumbra associated with the injured organ. In some embodiments, the H-NOX is delivered to the individual by bolus systemically followed by infusion to the injured organ or to the hypoxic penumbra associated with the injured organ.

In some embodiments, the H-NOX is administered at a dose of about 10 mg/kg to about 300 mg/kg. In some embodiments, the H-NOX is administered at a dose ranging from any of about 10 mg/kg to about 50 mg/kg, about 50 mg/kg to about 100 mg/kg, about 100 mg/kg to about 150 mg/kg, about 150 mg/kg to about 200 mg/kg, about 200 mg/kg to about 250 mg/kg, or about 250 mg/kg to about 300 mg/kg. In some embodiments, the H-NOX is delivered in a volume of about 10 ml to about 1 L. In some embodiments, the H-NOX is delivered in a volume of about 10 ml to about 25 ml, about 25 ml to about 50 ml, about 50 ml to about 100 ml, about 100 ml to about 200 ml, about 200 ml to about 300 ml, about 300 ml to about 400 ml, about 400 ml to about 500 ml, about 500 ml to about 600 ml, about 600 ml to about 700 ml, about 700 ml to about 800 ml, about 800 ml to about 900 ml, or about 900 ml to about 1 L. In some embodiments, the H-NOX is delivered as a bolus over a period of less than any of 1 minute, 2 minutes, 3 minutes, 4 minutes, 5 minutes, 10 minutes, 15 minutes, 20 minutes, 25 minutes, or about 30 minutes. In some embodiments, the H-NOX is delivered by infusion over a period of about 30 minutes to about 7 days. In some embodiment, the H-NOX is delivered by infusion over more than about any of 30 minutes, 1 hour, 2 hours, 3 hours, 4 hours, 5 hours, 6 hours, 7 hours, 8 hours, 9 hours, 10 hours, 11 hours, 12 hours, 16 hours, 18 hours, 1 day, 2 days, 3 days, 4 days, 5 days, 6 days, or 7 days. In some embodiments, the H-NOX protein is administered by infusion more than about any of 30 minutes to 1 hour, about 1 hour to about 2 hours, about 2 hours to about 3 hours, about 3 hours to about 4 hours, about 4 hours to about 5 hours, about 5 hours to about 6 hours, about 6 hours to about 7 hours, about 7 hours to about 8 hours, about 8 hours to about 9 hours, about 9 hours to about 10 hours, about 10 hours to about 11 hours, about 11 hours to about 12 hours, about 12 hours to about 16 hours, about 16 hours to about 18 hours, about 18 hours to about 1 day, about 1 day to about 2 days, about 2 days to about 3 days, about 3 days to about 4 days, about 4 days to about 5 days, about 5 days to about 6 days, or about 6 days to about 7 days. In some embodiments, the H-NOX is PEGylated. In some embodiments, the H-NOX is not PEGylated. In some embodiments, the H-NOX comprises PEGylated and non-PEGylated H-NOX.

In some embodiments, an effective amount of an H-NOX protein for administration to a human is between a few grams to over about 350 grams. Other exemplary doses of an H-NOX protein include about any of 4.4, 5, 10, or 13 g/dL (where g/dL is the concentration of the H-NOX protein solution prior to infusion into the circulation) at an appropriate infusion rate, such as about 0.5 ml/min (see, for example, Winslow, R. Chapter 12 In Blood Substitutes). It will be appreciated that the unit content of active ingredients contained in an individual dose of each dosage form need not in itself constitute an effective amount since the necessary effective amount could be reached by the combined effect of a plurality of administrations. The selection of the amount of an H-NOX protein to include in a pharmaceutical composition depends upon the dosage form utilized, the condition being treated, and the particular purpose to be achieved according to the determination of the ordinarily skilled artisan in the field. In some embodiments, the 1-NOX is PEGylated. In some embodiments, the H-NOX is not PEGylated. In some embodiments, the H-NOX comprises PEGylated and non-PEGylated H-NOX.

In some embodiments, a sustained continuous release formulation of the composition is used. Administration of an H-NOX protein can occur, e.g., for a period of seconds to hours depending on the purpose of the administration. For example, as a blood delivery vehicle, an exemplary time course of administration is as rapid as possible. Other exemplary time courses include about any of 10, 20, 30, 40, 60, 90, or 120 minutes Exemplary infusion rates for H-NOX solutions as blood replacements are from about 30 mL/hour to about 13,260 mL/hour, such as about 100 mL/hour to about 3,000 mL/hour. An exemplary total dose of H-NOX protein is about 900 mg/kg administered over 20 minutes at 13,260 mL/hour. An exemplary total dose of H-NOX protein for a swine is about 18.9 grams.

Exemplary dosing frequencies include, but are not limited to, at least 1, 2, 3, 4, 5, 6, or 7 times (i.e., daily) a week. In some embodiments, an H-NOX protein is administered at least 2, 3, 4, or 6 times a day. The H-NOX protein can be administered, e.g., over a period of a few days or weeks. In some embodiments, the H-NOX protein is administered for a longer period, such as a few months or years. The dosing frequency of the composition may be adjusted over the course of the treatment based on the judgment of the administering physician

In some embodiments of the invention, the H-NOX protein (e.g. a polymeric H-NOX protein) is used in combination or in conjunction with another therapy. In some embodiments, the H-NOX is administered to the individual any of at least about 1, 2, 3, 4, 5, 6, 12 or 24 hours before administration of the other therapy. In some embodiments, the H-NOX is administered to the individual at the same time as administration of the other therapy. In some embodiments, the H-NOX is administered to the individual any of at least about 1, 2, 3, 4, 5, 6, 12 or 24 hours after administration of the other therapy. In some embodiments, PEGylated H-NOX is administered in combination with another therapy. In some embodiments, non-PEGylated H-NOX is administered in combination with another therapy. In some embodiments, PEGylated and non-PEGylated H-NOX is administered to an individual in combination with another therapy.

As noted above, the selection of dosage amounts for H-NOX proteins depends upon the dosage form utilized, the frequency and number of administrations, the condition being treated, and the particular purpose to be achieved according to the determination of the ordinarily skilled artisan in the field. In some embodiments, an effective amount of an H1-NOX protein for administration to human is between a few grams to over 350 grams.

In some embodiments, two or more different H-NOX proteins are administered simultaneously, sequentially, or concurrently. In some embodiments, another compound or therapy useful for the delivery of O₂ is administered simultaneously, sequentially, or concurrently with the administration of one or more H-NOX proteins.

7.5.1. Administration of H-NOX Proteins in Combination with Catecholamines

In a specific embodiment, any H-NOX protein or a mixture of H-NOX proteins described herein is used for any therapeutic indications described herein in combination with a catecholamine (e.g., epinephrine or norepinephrine). In a certain embodiment, an H-NOX protein or a mixture of H-NOX proteins is in a pharmaceutical composition with a catecholamine. In another embodiment, an H-NOX protein or a mixture of H-NOX proteins is not in the same composition as a catecholamine, but is administered in combination with a catecholamine.

Doses, dosage regimens and modes of administration of catecholamines that can be used for the therapeutic indications described herein are known in the art. In one embodiment, epinephrine is used in the compositions or methods provided herein in an amount from 0.1 mg to 2 mg, from 0.2 mg to 1 mg, or from 0.5 mg to 1 mg, or infused in an amount from 0.05 to 2 mcg/kg/min, or from 0.1 to 0.5 mcg/kg/min. In one embodiment, epinephrine is used in the compositions or methods provided herein in an amount from 0.5 to 1.5 mg (e.g., 1 mg), for example, for intravenous administration every 3-5 minutes (e.g., for the treatment of a human adult). In one embodiment, epinephrine is administered in an amount from 0.01 to 0.03 mg/kg (e.g., for the treatment of a human child). In one embodiment, epinephrine is infused (e.g., as a continuous intravenous drip) in an amount from 2 to 10 mcg/min (e.g., wherein the subject being treated has bradycardia). In one embodiment, epinephrine is infused in an amount from 0.1 to 0.5 mcg/kg/min (e.g., wherein the subject being treated has hypotension following cardiac or pulmonary arrest). In one embodiment, atropine is used in the compositions and methods provided herein in an amount from 0.25 to 1 mg (e.g., 0.5 mg), for example, for intravenous administration every 3-5 minutes (e.g., for the treatment of a human adult). In one embodiment, atropine is administered in an amount from 0.01 to 0.05 mg/kg (e.g., 0.02 mg/kg), for example, intravenously every 3-5 minutes (e.g., for the treatment of a human child). In one embodiment, norepinephrine is infused in an amount from 0.1 to 3.3 mcg/kg/min, from 0.1 to 1.5 mcg/kg/min, from 0.2 to 1.3 mcg/kg/min, or from 0.1 to 0.5 mcg/kg/min. In certain embodiments, a catecholamine (e.g., epinephrine or norepinephrine) is administered intravenously, subcutaneously, intramuscularly, intracardially, or endotracheally. In one embodiment, a catecholamine (e.g., epinephrine or norepinephrine) is administered intravenously.

In certain embodiments, the H-NOX protein (or a mixture of H-NOX proteins) is administered to a subject before, concurrently or after administration of a catecholamine (e.g., epinephrine or norepinephrine). In one embodiment, the H-NOX protein (or a mixture of H-NOX proteins) and a catecholamine (e.g., epinephrine or norepinephrine) are administered concurrently. In particular embodiments, the H-NOX protein (or a mixture of H-NOX proteins) is administered within 24 hours, 23 hours, 22 hours, 21 hours, 20 hours, 19 hours, 18 hours, 17, hours, 16 hours, 15 hours, 14 hours, 13 hours, 12 hours, 11 hours, 10 hours, 9 hours, 8 hours, 7 hours, 6 hours, 5 hours, 4 hours, 3 hours, 2 hours, 1 hour, 45 minutes, 30 minutes, 20 minutes, 15 minutes, 10 minutes, or 5 minutes, of the administration of a catecholamine (e.g., epinephrine or norepinephrine). In some embodiments, an H-NOX protein or a mixture of H-NOX proteins is administered to an individual any of at least about 1, 2, 3, 4, 5, 6, 12 or 24 hours before administration of a catecholamine. In some embodiments, an H-NOX protein or a mixture of H-NOX proteins is administered to the individual at the same time as administration of a catecholamine. In some embodiments, an H-NOX protein or a mixture of H-NOX proteins is administered to the individual any of at least about 1, 2, 3, 4, 5, 6, 12 or 24 hours after administration of a catecholamine.

Exemplary dosing frequencies of an H-NOX protein (or a mixture of H-NOX proteins) and/or a catecholamine include, but are not limited to, at least 1, 2, 3, 4, 5, 6, or 7 times (i.e., daily) a week. In some embodiments, an H-NOX protein (or a mixture of H-NOX proteins) and/or a catecholamine are administered at least 2, 3, 4, or 6 times a day. In particular embodiments, the H-NOX protein (or a mixture of H-NOX proteins) is administered in combination with a catecholamine (e.g., epinephrine or norepinephrine) once a day, once or twice a week, once or twice in two weeks, or once or twice a month. An H-NOX protein (or a mixture of H-NOX proteins) and/or a catecholamine can be administered, e.g., over a period of a few days or weeks. In some embodiments, an H-NOX protein (or a mixture of H-NOX proteins) and/or a catecholamine are administered for a longer period, such as a few months or years. In particular embodiments, the H-NOX protein (or a mixture of H-NOX proteins) is administered in combination with a catecholamine (e.g., epinephrine or norepinephrine) for at least, or more than, 1 week, 2 weeks, 3 weeks, 4 weeks, 5 weeks, 6 weeks, 7 weeks, 8 weeks, 10 weeks, 3 months, 4 months, 5 months, 6 months, 8 months or 1 year. In other particular embodiments, a subject is administered a single dose of the H-NOX protein (or a mixture of H-NOX proteins) in combination with one dose of a catecholamine, optionally followed by subsequent doses of a catecholamine (e.g., epinephrine or norepinephrine).

7.6. Kits with H-NOX Proteins

Also provided are articles of manufacture and kits that include any of the H-NOX proteins described herein including polymeric H-NOX proteins, and suitable packaging. In some embodiments, the invention includes a kit with (i) a H-NOX protein (such as a wild-type or mutant H-NOX protein described herein or formulations thereof as described herein) and (ii) instructions for using the kit to deliver O₂ to an individual.

Also provided are articles of manufacture and kits that include any of the H-NOX proteins or mixtures of H-NOX proteins described herein, any of the catecholamines described herein, and suitable packaging. In some embodiments, the invention includes a kit with (i) an H-NOX protein (or a mixture of H-NOX proteins), (ii) a catecholamine (e.g., epinephrine or norepinephrine) and, optionally, (iii) instructions for using the kit to deliver O₂ to an individual. In a specific embodiment, the H-NOX protein(s) are in a separate container from the catecholamine. In a specific embodiment, an H-NOX protein (or a mixture of H-NOX proteins) and a catecholamine (e.g., epinephrine or norepinephrine) are in the same composition.

In some embodiments, kits are provided for use in treatment of any disorder or condition described herein in an individual.

In some embodiments, the kits comprise both PEGylated and non-PEGylated H-NOX. In some embodiments, the PEGylated H-NOX and non-PEGylated H-NOX in the kit are in a composition. In some embodiments, the ratio of PEGylated to non-PEGylated H-NOX in the composition is any of about 99:1, 95:5.90:10, 85:15, 80:20, 75:25, 70:30, 65:35, 60:40; 55:45, 50.50, 45:55, 40:60, 35.65, 30:70, 25:75, 20:80, 15:85; 10.90; 5:95, 1.99 or any ratio therebetween.

In some embodiments, the kit comprises a polymeric H-NOX protein (e.g., a PEGylated polymeric H-NOX protein and/or a non-PEGylated H-NOX protein). In some embodiments, the kit comprises an effective amount of a polymeric H-NOX protein comprising two or more wild-type or mutant H-NOX domains. In some embodiments, the kit comprises an effective amount of a recombinant monomeric H-NOX protein comprising a wild-type or mutant 1-NOX domain and a polymerization domain as described herein. In some embodiments, the kit comprises a trimeric H-NOX protein comprising three monomers, each monomer comprising a mutation corresponding to a T. tengcongensis L144F H-NOX mutation and a trimerization domain. In some embodiments, the trimeric H-NOX protein comprises human H-NOX domains. In some embodiments, the trimeric H-NOX protein comprises canine H-NOX domains. In some embodiments, the kit comprises a trimeric H-NOX protein comprising three monomers, each monomer comprising a T. tengcongensis L144F H-NOX domain and a foldon domain. In some embodiments, the kit comprises a trimeric H-NOX protein comprising three monomers, each monomer comprising a T. tengcongensis L144F H-NOX domain and a foldon domain.

Suitable packaging for compositions described herein are known in the art, and include, for example, vials (e.g., sealed vials), vessels, ampules, bottles, jars, flexible packaging (e.g., sealed Mylar or plastic bags), and the like. These articles of manufacture may further be sterilized and/or sealed. Also provided are unit dosage forms comprising the compositions described herein. These unit dosage forms can be stored in a suitable packaging in single or multiple unit dosages and may also be further sterilized and sealed. Instructions supplied in the kits of the invention are typically written instructions on a label or package insert (e.g., a paper sheet included in the kit), but machine-readable instructions (e.g., instructions carried on a magnetic or optical storage disk) are also acceptable. The instructions relating to the use of H-NOX proteins generally include information as to dosage, dosing schedule, and route of administration for the intended treatment or industrial use. The kit may further comprise a description of selecting an individual suitable or treatment. In some embodiments, the kit comprises instructions for treatment of any disorder or condition described herein.

The containers may be unit doses, bulk packages (e.g., multi-dose packages) or sub-unit doses. For example, kits may also be provided that contain sufficient dosages of H-NOX proteins disclosed herein to provide effective treatment for an individual for an extended period, such as about any of a week, 2 weeks, 3 weeks, 4 weeks, 6 weeks, 8 weeks, 3 months, 4 months, 5 months, 6 months, 7 months, 8 months, 9 months, or more. Kits may also include multiple unit doses of H-NOX proteins and instructions for use and packaged in quantities sufficient for storage and use in pharmacies, for example, hospital pharmacies and compounding pharmacies. In some embodiments, the kit includes a dry (e.g., lyophilized) composition that can be reconstituted, resuspended, or rehydrated to form generally a stable aqueous suspension of H-NOX protein.

In some embodiments, the invention provides an article of manufacture containing a PEGylated and/or a non-PEGylated H-NOX In some embodiments, the invention provides an article of manufacture containing a mixture of PEGylated and non-PEGylated H-NOX. In some embodiments, the article of manufacture is a vessel, a vial, ajar, an ampule, a capsule, a syringe, or a bag. In some embodiments, the weight ratio of PEGylated H-NOX to non-PEGylated H-NOX is any of about 99:1, about 95:5, about 90:10, about 80:20, about 75:25, about 70:30, about 60:40, about 50:50, about 40:60, about 30:70, about 25:75, about 20:80, about 10.90, about 5:95, or about 1:99 In some embodiments, the PEGylated H-NOX is sequestered from the non-PEGlyated H-NOX by a barrier. In some embodiments the barrier may be removed prior to use to allow the PEGylated H-NOX and the non-PEGylated H-NOX to mix. In some embodiments, the invention provides a syringe containing a mixture of PEGylated and non-PEGylated H-NOX.

In some embodiments, the invention provides an article of manufacture containing (i) a PEGylated and/or a non-PEGylated H-NOX and (ii) a catecholamine. In some embodiments, the invention provides an article of manufacture containing (i) a mixture of PEGylated and non-PEGylated H-NOX and (ii) a catecholamine. In some embodiments, the article of manufacture is a vessel, a vial, ajar, an ampule, a capsule, a syringe, or a bag. In some embodiments, the weight ratio of PEGylated H-NOX to non-PEGylated H-NOX is any of about 99:1, about 95:5, about 90:10, about 80:20, about 75:25, about 70.30, about 60:40, about 50:50, about 40:60, about 30:70, about 25:75, about 20:80, about 10:90, about 5:95, or about 1:99. In some embodiments, the PEGylated H-NOX and/or non-PEGylated H-NOX is sequestered from the catecholamine by a barrier. In some embodiments the barrier may be removed prior to use to allow the H-NOX and the catecholamine to mix. In some embodiments, the invention provides a syringe containing a mixture of PEGylated and non-PEGylated H-NOX and a catecholamine.

### 7.7 Exemplary Methods for Production of H-NOX Proteins

The present invention also provides methods for the production of compositions comprising PEGylated and non-PEGylated H-NOX proteins, the method comprising mixing PEGylated H-NOX with non-PEGylated H-NOX. In some embodiments, the weight ratio of PEGylated to non-PEGylated H-NOX in the composition is any of about 99.1, 95:5, 90:10, 85:15, 80:20, 75:25, 70:30, 65:35, 60:40; 55:45, 50:50, 45:55, 40:60, 35:65, 30:70, 25:75, 20:80, 15:85; 10:90; 5:95; 1:99 or any ratio therebetween.

The present invention also provides methods for the production of any of the H-NOX proteins (e.g., polymeric H-NOX proteins) described herein. In some embodiments, the method involves culturing a cell that has a nucleic acid encoding a polymeric H-NOX protein under conditions suitable for production of the polymeric H-NOX protein. In various embodiments, the polymeric H1-NOX is also purified (such as purification of the H1-NOX protein from the cells or the culture medium). In some embodiments, the method involves culturing a cell that has a nucleic acid encoding a monomer H-NOX protein comprising an H-NOX domain and a polymerization domain. The monomers then associate in vivo or in vitro to form a polymeric H-NOX protein. A polymeric H-NOX protein comprising heterologous H-NOX domains may be generated by co-introducing two or more nucleic acids encoding monomeric H-NOX proteins with the desired H-NOX domains and where in the two or more monomeric H-NOX proteins comprise the same polymerization domain.

In some embodiments, a polymeric H-NOX protein comprising heterologous H-NOX domains is prepared by separately preparing polymeric H-NOX proteins comprising homologous monomeric H-NOX subunits comprising the desired H-NOX domains and a common polymerization domain. The different homologous H-NOX proteins are mixed at a desired ratio of heterologous H-NOX subunits, the homologous polymeric H-NOX proteins are dissociated (e.g. by heat, denaturant, high salt, etc.), then allowed to associate to form heterologous polymeric H-NOX proteins. The mixture of heterologous polymeric H-NOX proteins may be further purified by selecting for the presence of the desired subunits at the desired ratio. For example, each different H-NOX monomer may have a distinct tag to assist in purifying heterologous polymeric H-NOX proteins and identifying and quantifying the heterologous subunits.

As noted above, the sequences of several wild-type H-NOX proteins and nucleic acids are known and can be used to generate mutant H-NOX domains and nucleic acids of the present invention. Techniques for the mutation, expression, and purification of recombinant H-NOX proteins have been described by, e.g., Boon, E. M. et al. (2005) Nature Chemical Biology 1:53-59 and Karow, D. S. et al (Aug. 10, 2004) Biochemistry 43(31):10203-10211, which is hereby incorporated by reference in its entirety, particularly with respect to the mutation, expression, and purification of recombinant H-NOX proteins. These techniques or other standard techniques can be used to generate any mutant H-NOX protein.

In particular, mutant H-NOX proteins described herein can be generated a number of methods that are known in the art. Mutation can occur at either the amino acid level by chemical modification of an amino acid or at the codon level by alteration of the nucleotide sequence that codes for a given amino acid. Substitution of an amino acid at any given position in a protein can be achieved by altering the codon that codes for that amino acid. This can be accomplished by site-directed mutagenesis using, for example: (i) the Amersham technique (Amersham mutagenesis kit, Amersham, Inc., Cleveland, Ohio) based on the methods of Taylor, J. W. et al. (Dec. 20, 1985) Nucleic Acids Res. 13(24):8749-8764; Taylor, J. W. et al. (Dec. 20, 1985) Nucleic Acids Res. 13(24):8765-8785; Nakamaye, K. L. et al. (Dec. 22, 1986) Nucleic Acids Res. 14(24) 9679-9698; and Dente et al. (1985) in DNA Cloning, Glover, Ed., IRL Press, pages 791-802, (ii) the Promega kit (Promega Inc., Madison, Wis.), or (iii) the Biorad kit (Biorad Inc., Richmond, Calif.), based on the methods of Kunkel, T A. (January 1985). Proc. Natl. Acad. Sci. USA 82(2):488-492; Kunkel, T. A. (1987) Methods Enzymol. 154:367-382; Kunkel, U.S. Pat. No. 4,873,192, which are each hereby incorporated by reference in their entireties, particularly with respect to the mutagenesis of proteins. Mutagenesis can also be accomplished by other commercially available or non-commercial means, such as those that utilize site-directed mutagenesis with mutant oligonucleotides.

Site-directed mutagenesis can also be accomplished using PCR-based mutagenesis such as that described in Zhengbin et al. (1992) pages 205-207 in PCR Methods and Applications, Cold Spring Harbor Laboratory Press, New York; Jones, D. H. et al. (February 1990) Biotechniques 8(2):178-183; Jones, D. H. et al. (January 1991) Biotechniques 10(1):62-66, which are each hereby incorporated by reference in their entireties, particularly with respect to the mutagenesis of proteins Site-directed mutagenesis can also be accomplished using cassette mutagenesis with techniques that are known to those of skill in the art.

A mutant H-NOX nucleic acid and/or polymerization domain can be incorporated into a vector, such as an expression vector, using standard techniques. For example, restriction enzymes can be used to cleave the mutant H-NOX nucleic acid and the vector. Then, the compatible ends of the cleaved mutant H-NOX nucleic acid and the cleaved vector can be ligated. The resulting vector can be inserted into a cell (e.g., an insect cell, a plant cell, a yeast cell, or a bacterial cell) using standard techniques (e.g., electroporation) for expression of the encoded H-NOX protein.

In particular, heterologous proteins have been expressed in a number of biological expression systems, such as insect cells, plant cells, yeast cells, and bacterial cells. Thus, any suitable biological protein expression system can be utilized to produce large quantities of recombinant H-NOX protein. In some embodiments, the H-NOX protein (e.g., a mutant or wild-type H-NOX protein) is an isolated protein.

If desired, H-NOX proteins can be purified using standard techniques. In some embodiments, the protein is at least about 60%, by weight, free from other components that are present when the protein is produced. In various embodiments, the protein is at least about 75%, 90%, or 99%, by weight, pure. A purified protein can be obtained, for example, by purification (e.g., extraction) from a natural source, a recombinant expression system, or a reaction mixture for chemical synthesis. Exemplary methods of purification include immunoprecipitation, column chromatography such as immunoaffinity chromatography, magnetic bead immunoaffinity purification, and panning with a plate-bound antibody, as well as other techniques known to the skilled artisan. Purity can be assayed by any appropriate method, e.g., by column chromatography, polyacrylamide gel electrophoresis, or HPLC analysis. In some embodiments, the purified protein is incorporated into a pharmaceutical composition of the invention or used in a method of the invention. The pharmaceutical composition of the invention may have additives, carriers, or other components in addition to the purified protein.

In some embodiments, the polymeric H-NOX protein comprises one or more His₆ tags. An H-NOX protein comprising at least one His₆ tag may be purified using chromatography; for example, using Ni²⁺-affinity chromatography. Following purification, the His₆ tag may be removed; for example, by using an exopeptidase. In some embodiments, the invention provides a purified polymeric H-NOX protein, wherein the polymeric H-NOX protein was purified through the use of a His₆ tag. In some embodiments, the purified H-NOX protein is treated with an exopeptidase to remove the His₆ tags.

### 8. EXAMPLE 1

### 8.1 Introduction

This Example describes the use of an H-NOX composition (O₂ delivery biotherapeutic), in particular OMX-CV, to alleviate hypoxia-induced tissue dysfunction in the heart. Derived from the heme-based nitric oxide (NO)/oxygen (H-NOX) sensing proteins found in the thermostable bacterium Thermoanaerobacter tengcongensis (TD) (Karow D. S. et al. (2004) Biochemistry 43:10203-10211), OMX-CV was engineered to increase circulation half-life, and alterations to the heme-binding pocket to finetune both selectivity and avidity of interaction with the diatomic gases NO and molecular O₂ Boon E. M. and Marletta M. A. (2005) Curr. Opin. Chem. Biol. 9:441-446, LeMoan N. et al. (2017) Neuroprotective Therapy for Stroke and Ischemic Disease 641-664). Unlike hemoglobin (Hb)-based O₂ delivery biotherapeutics that scavenged NO and therefore triggered significant vascular sequelae, including hypertension, renal dysfunction, and increased risk of myocardial infarction and death (Chen J. Y et al. (2009) Clinics (Sao Paulo) 64:803-813; Natanson C et a. (2008) JAMA 299:2304-2312; Olson J. S. et al. (2004) Free Radic. Rio. Med. 36:685-697), the protein component of OMX-CV is uniquely tuned to bind molecular O₂ in a way that reduces NO reactivity 50-fold compared with Hb (LeMoan N. et al. (2017) Neuroprotective Therapy for Stroke and Ischemic Disease 641-664), alleviating the potential risk of vasoconstriction.

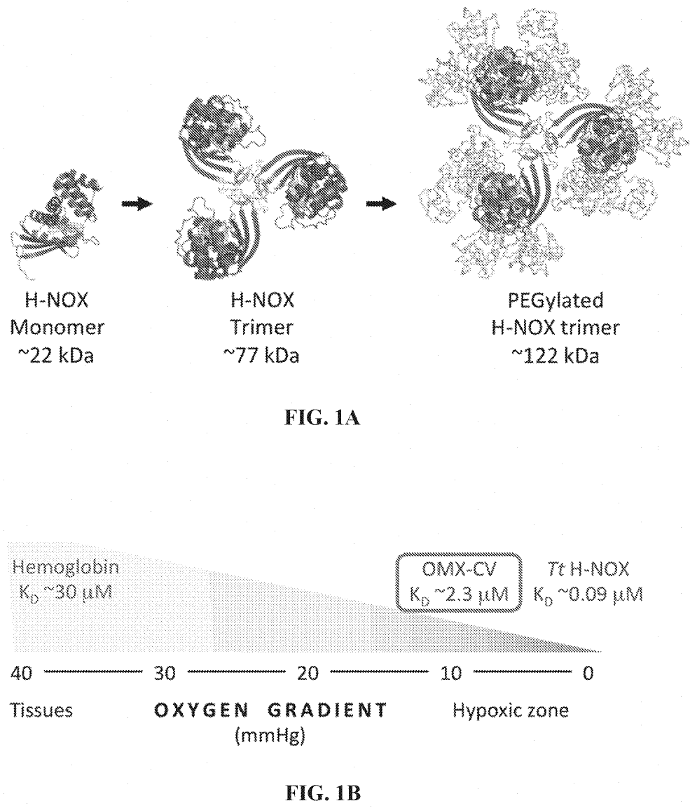

Additionally, relative to Hb, the protein component of OMX-CV binds to O₂ with a very high affinity, exhibiting a dissociation constant (K_{D}) of about 2.4 μM (LeMoan N. et al. (2017) Neuroprotective Therapy for Stroke and Ischemic Disease 641-664). FIG. 1B shows a schematic comparing the O₂ affinities of wild-type Tt H-NOX and OMX-CV with that of Hb, and illustrates how OMX-CV can effectively deliver O₂ only to tissues that are significantly hypoxic while bypassing those at physiologic O₂ tensions. Following O₂ delivery within the hypoxic capillary environment, the unbound OMX-CV molecules enter the systemic venous and pulmonary vascular beds. In this manner, OMX-CV circulates and can be predicted to sustain an ongoing, targeted O₂ delivery to the most hypoxic organs and tissues without unnecessary and potentially harmful (Helmerhorst H. J. et al. (2015) Crit. Care 19:284) oxygenation of tissues at physiologic O₂ tensions.

In order to test the hypothesis that in the setting of severe myocardial hypoxia, OMX-CV administration would increase O₂ delivery to the heart and improve cardiac mechanical function, a juvenile lamb model of severe acute alveolar hypoxia was utilized. The lamb is a robust large animal model that has been extensively utilized because of its close approximation of human cardiovascular function (Rudolph A. M. (2009) Wiley-Blackwell p. 538). This Example presents data regarding the safety and efficacy of OMX-CV administration in the setting of systemic hypoxia supporting the use of OMX-CV as a promising O₂ delivery biotherapeutic. In particular, the utility of OMX-CV as an O₂ delivery biotherapeutic for the hypoxic myocardium was tested.

This Example shows that, in OMX-CV-treated animals, myocardial oxygenation was improved without negatively impacting systemic or PVR, and both right ventricle (RV) and left ventricle (LV) contractile function were maintained at pre-hypoxic baseline levels. These data show that OMX-CV is a promising and safe O₂ delivery biotherapeutic for the preservation of myocardial contractility in the setting of acute hypoxia. In addition, this Example demonstrates that OMX-CV can effectively deliver oxygen to a lamb heart with induced severe hypoxia, without overexposing the animal to oxygen or triggering systemic vascular reactivity

In addition, this Example shows that OMX-CV-treated animals exhibit preserved contractility despite smaller increases in catecholamine levels (relative to vehicle-treated animals). The improved myocardial performance in the presence of lower induction of catecholamines suggests a greater capacity of the H-NOX-treated animals to respond to adrenergic signaling under hypoxic stress.

“OMX-CV” as used in this Example refers to a 1:1 mixture (by weight) of an H-NOX protein covalently bound to polyethylene glycol (PEG) and an H-NOX protein not bound to PEG, wherein the H-NOX protein (both the protein bound to PEG and the protein not bound to PEG) is a trimeric H-NOX protein comprising three monomers, wherein each of the three monomers comprises a T. tengcongensis H-NOX domain covalently linked to a trimerization domain, wherein the trimerization domain is a foldon domain of bacteriophage T4 fibritin (having the amino acid sequence of SEQ ID NO:4 set forth herein, wherein the T. tengcongensis H-NOX domain has an L144F amino acid substitution relative to the amino acid sequence of SEQ ID NO:2 set forth herein, and wherein the trimeric H-NOX protein comprises three PEG molecules per monomer, wherein each of the three PEG molecules is a linear methoxy PEG (m-PEG) having a molecular weight of about 5 kDa, and wherein each of the three monomers has the amino acid sequence of SEQ ID NO:8 set forth herein. As will be understood by a person skilled in the art, the three PEG molecules per monomer is an average number of PEG molecules per monomer.

### 8.2 Materials and Methods

In the study presented in this Example, juvenile lambs were sedated, mechanically ventilated, and instrumented to measure cardiovascular parameters. Biventricular admittance catheters were inserted to perform pressure-volume (PV) analyses. Systemic hypoxia was induced by ventilation with 10% O₂. Following 15 minutes of hypoxia, the lambs were treated with OMX-CV (200 mg/kg IV) or vehicle. Acute hypoxia induced significant increases in heart rate (HR), pulmonary blood flow (PBF), and pulmonary vascular resistance (PVR) (p<0.05). At 1 hour, vehicle-treated lambs exhibited severe hypoxia and a significant decrease in biventricular contractile function. The Materials and Methods used in this Example are described in more detail below:

Surgeries: In this Example, 13 juvenile lambs (4-6 weeks of age) were anesthetized with fentanyl, ketamine, and diazepam and paralyzed with vecuronium to facilitate intubation and mechanical ventilation. Ongoing sedation and neuromuscular blockade were administered as a continuous infusion of ketamine, fentanyl, diazepam, and vecuronium. The sedative mixture was titrated to maintain age-appropriate HR. Femoral venous and arterial access were obtained via cutdown of the hind limbs, and arterial pressure was continuously transduced and recorded. The animals were ventilated with 21% FiO2 initially, with a positive end expiratory pressure of 5 cm H₂O, tidal volumes of 10 mL/kg, and respiratory rate titrated to maintain pCO2 of 35-45 millimeters mercury (mmHg) by arterial blood gas measurements. Thoracotomy was performed and Sorenson Neonatal Transducers (Abbott Critical Care Systems, N Chicago, Ill.) were introduced into the left and right atria and main pulmonary artery (MPA) to continually transduce and record pressures. An ultrasonic flow probe (Transonics Sytems, Ithaca, N.Y.) was placed on the left pulmonary artery (LPA) to continuously monitor and record blood flow. Admittance PV catheters (Transonics Systems, Ithaca. N.Y.) were introduced into the RV and LV via ventriculostomy to perform ventricular pressure volume analysis. These catheters consist of a solid-state sensor that directly measures pressure with high precision and excitation and recording electrodes that measure volume based on electrical admittance. Alternating current applied to the excitation electrodes generates an electrical field within the ventricle and the recording electrodes measure voltage changes, allowing calculation of resistance and conductance. With input of a measured blood resistivity and baseline stroke volume (as assessed by total cardiac output estimate from LPA flow/HR), time varying conductance can be used to solve for ventricular blood volume in real time (Porterfield J. E. et al. (2009) J. Appl. Physiol. 107:1693-1703). Animals with Hb levels of less than 7.5 g/dL following surgical instrumentation were transfused with fresh whole maternal blood in increments of 5 mL/kg to achieve this minimum threshold.

Following instrumentation, the animals were allowed to recover to steady state until they required no further adjustment to sedatives and exhibited stable hemodynamic parameters. This time was designated as the normoxic baseline and blood gas analysis was performed. Baseline ventricular end systolic pressure-volume relationship (ESPVR) was assessed by transient IVC occlusion. Following baseline assessment, the animals were subjected to sustained alveolar hypoxia by ventilation with an admixture of atmospheric gas and nitrogen to achieve a FiO2 of 10%. Arterial blood gas analysis was performed every 15 minutes with blood withdrawn from the femoral artery and analyzed using a Radiometer ABL5 pH/blood gas analyzer (Radiometer, Copenhagen, Denmark). Ventilatory rate was adjusted to maintain PCO₂ 35-45 mmHg and metabolic acidosis was corrected with NaHCO₃boluses to maintain pH>7.30.

Animal care and use: All protocols and procedures for this work were approved by the Institutional Animal Care and Use Committee of the University of California, San Francisco. Animals' vital signs, including core temperature, were monitored throughout the study, and they were given intravenous fluids and prophylactic antibiotics per protocol. At the end of each protocol, all lambs were euthanized with a lethal injection of sodium pentobarbital followed by bilateral thoracotomy, as described in the NIH Guidelines for the Care and Use of Laboratory Animals.

OMX-CV production: The engineered Tt H-NOX L144F protein described in this Example was produced by QuikChange Site-Directed Mutagenesis (Agilent), subcloned into an expression plasmid, transformed into Escherichia coli, and expressed essentially as described in Karow D. S. et al. (2004) Biochemistry 43.10203-10211. Cells were harvested by hollow-fiber tangential-flow filtration and processed immediately. The His-tagged TI H-NOX L144F protein was purified from cell lysate using Ni-affinity chromatography and further polished by passage over an anion-exchange column to remove remaining host cell DNA, host cell proteins, and endotoxins. The purified protein was formulated to produce OMX-CV, and frozen at −80° C. until use. Protein concentrations were determined using UV-Vis spectrophotometry as described in Karow D. S. et al. (2004) Biochemistry 43:10203-10211. Prior to use in animal studies, OMX-CV was subjected to purity testing by SDS-PAGE (Invitrogen) and SEC-HPLC (Agilent) and safety testing by kinetic chromogenic LAL test for endotoxin (Charles River Laboratories). For use in animal studies, proteins lots were required to be greater than 95% pure and have endotoxin levels less than 0.1 EU/mg.

OMX-CV administration: After 15 minutes of alveolar hypoxia, the animals received either 200 mg/kg of OMX-CV (about 4 mL/kg by volume) as a bolus over 10 minutes, followed by continuous infusion at 70 mg/kg/hour (OMX-CV group, n=6), or an equivalent volume of the OMX-CV vehicle solution administered in the same manner (control group, n=7). At 60 minutes of alveolar hypoxia, repeat evaluation of the ESPVR was assessed by IVC occlusion.

Physiologic monitoring: Physiologic data were continuously recorded and analyzed using the Ponemah Physiology Platform (Data Sciences International. New Brighton, Minn.) with Acquisition Interface, ACQ-7700 (Data Sciences International, St. Paul, Minn.). For calculation of total cardiac output, LPA blood flow was assumed to represent 45% of total output, as previously established in juvenile lambs by Rudolph A. M. (2009) Wiley-Blackwell p. 538. This was indexed to animal size by dividing by the animal's body weight in kilograms. PVR was calculated as the difference of mean pulmonary arterial pressure and left atrial pressure divided by the indexed cardiac output. SVR was calculated as the difference of mean systemic arterial pressure and right atrial pressure divided by the indexed cardiac output. Pressure volume loop recording and analysis were performed using Labscribe software (iWorx, Dover, N.H.).

Epinephrine and norepinephrine ELISA: At baseline and again at 60 minutes of hypoxia, plasma and serum samples were collected from all animals (control group, n=7 and OMX-CV group, n=6) for additional analysis, including measurement of circulating catecholamines Determination of epinephrine and norepinephrine levels in plasma was performed using a colorimetric ELISA kit (ABNOVA) according to the manufacturer's instructions.

Pimonidazole ELISA: In a subset of animals (control group, n=3 and OMX-CV group, n=3), following the final physiologic assessment, pimonidazole (85 mg/kg) was administered intravenously over 10-15 minutes, as tolerated. Thirty minutes following the pimonidazole infusion, the animals were euthanized for tissue collection. Myocardial tissues were snap-frozen and proteins were then extracted and processed for competitive pimonidazole ELISA, as described in Kleiter M. M. et al. (2006) Int. J. Radiat. Oncol. Biol. Phys. 64:592-602. Standard curves for the pimonidazole ELISA were fit using a five-parameter logistic equation and used to determine 1Co values. Values were normalized to the protein concentration in each sample and then expressed relative to the vehicle control

Immunohistochemistry of pimonidazole and OMX-CV: Myocardial tissues were frozen in OCT and processed for cryosectioning, followed by immunohistochemical analysis. Sections were fixed with 100% methanol for 20 minutes at −20° C., then blocked and permeabilized with 5% BSA, 5% goat serum, and 0.1% Tween 20 for 1-2 hours at room temperature. Sections were then incubated with anti-pimonidazole (Hypoxyprobe, 1.100), anti-OMX-CV (1:200, Mouse monoclonal) antibodies overnight at 4° C., followed by anti-rabbit or anti-mouse secondary antibodies (1:1,000, Jackson Immunoresearch Laboratories, West Grove, Pa.) for 2 hours at room temperature. The sections were mounted in SlowFade DAPI (Invitrogen) and imaged at the UCSF Laboratory for Cell Analysis Core with an HD AxioImager Zeiss microscope equipped with a CCD digital camera.

Statistical analysis: Comparison of physiologic data comparing pre-hypoxic baseline to the first hypoxic physiologic time point was performed using a paired Student t test. Evaluation of cardiac output over the duration of the study between groups was performed using two-way ANOVA analysis. Evaluation of PVR and SVR before and after treatment between groups was performed using two-way ANOVA analysis. Pimonidazole levels were compared between groups using an unpaired Student t test. For ESPVR data, the slope of the ESPVR at 60 minutes of hypoxia for each ventricle of each animal was normalized to its own baseline ESPVR. These normalized values were then compared between groups using an unpaired Student t test. Epinephrine and norepinephrine levels at 60 minutes of hypoxia were compared between groups using unpaired Student t test. For all statistical tests performed, p≤0.05 was considered to be significant. All analyses were performed using GraphPad Prism version 6.04 for Macintosh, Graph-Pad Software, La Jolla, Calif.

### 8.3 Results

### 8.3.1 Acute Alveolar Hypoxia Induces a Dramatic Physiologic Response.

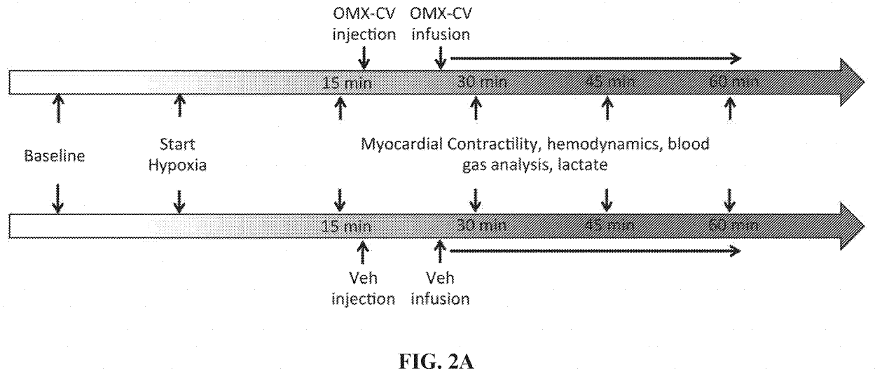

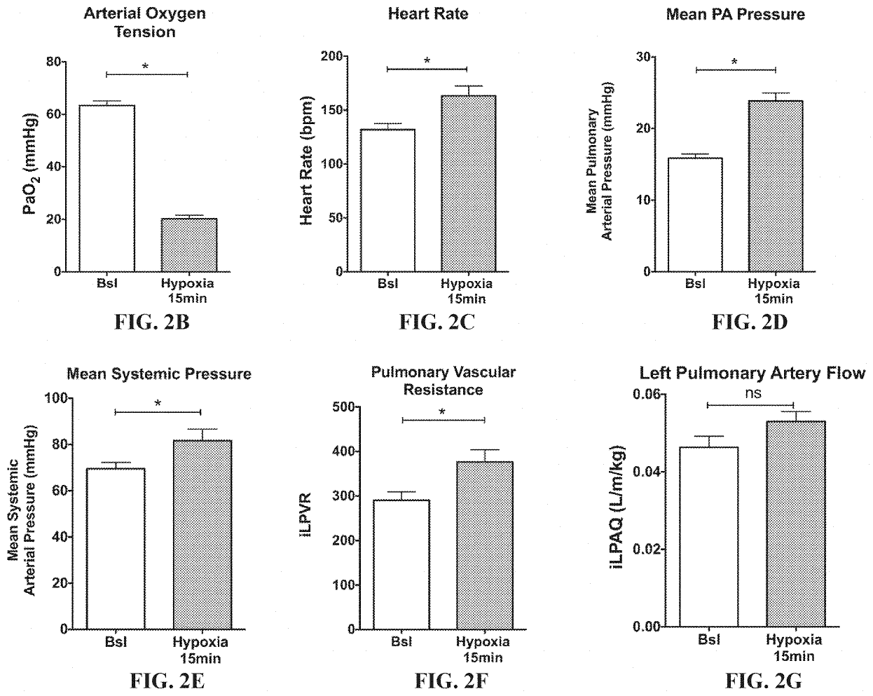

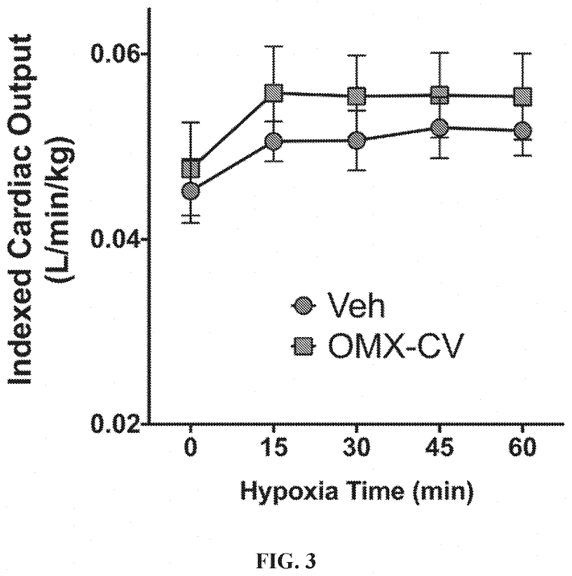

The acute cardiovascular response to progressive alveolar hypoxia in large animal models has been described by others (Kontos H. A et al., (1965) Am. J. Physiol. 209:397-403; Downing S. E. et al. (1969) Am. J. Physiol. 217: 728-735). In this Example, a model of acute alveolar hypoxia in juvenile lambs triggered via inhalation of a gas mixture containing 10% O₂ was established (FIG. 2A). The acute cardiovascular response to progressive alveolar hypoxia in large animal models has been described by others (Kontos H. A. et al., (1965) Am. J. Physiol. 209:397-403; Downing S. E. et al. (1969) Am. J. Physiol. 217: 728-735) In the present a model of acute alveolar hypoxia in juvenile lambs, physiologic data were compared at pre-hypoxic baseline and at 15 minutes following institution of hypoxic ventilation (prior to experimental intervention) for all animals included in the analysis (n=13). A precipitous fall in arterial O₂ tension (PaO₂) was noted with the onset of alveolar hypoxia that was then sustained for the duration of the study (FIG. 2B). FIGS. 2C-2F demonstrate the dramatic changes in physiologic parameters that accompany this severe hypoxemia at 15-minute following institution of hypoxic ventilation. All the animals exhibited acute increases in heart rate (HR) systemic blood pressure (systolic and mean), pulmonary blood pressure (systolic, diastolic, and mean), and left and right atrial pressures. There was also a significant increase in pulmonary vascular resistance (PVR) attributable to hypoxic pulmonary vasoconstriction (see also Moudgil R. et al. (2005) J. App Physiol. (1985) 98:390-403). However, there was no significant alteration in either the systemic diastolic blood pressure or the systemic vascular resistance (SVR). Additionally, there was an overall increase in cardiac output of approximately 150 (FIG. 2G). Although this increase just failed to reach statistical significance when evaluated at the 15-minute following institution of hypoxic ventilation (p=0.063), there was a significant increase in cardiac output amongst all animals (but no between-group difference) when evaluated over the duration of the hypoxic exposure (FIG. 3). Table 2 provides additional cardiovascular physiologic parameters comparing OMX-CV and vehicle groups at their respective hypoxic baselines (before drug) and study conclusion (60 minutes).

TABLE 2
Compilation of cardiovascular physiologic parameters measured
during hypoxic conditions in lambs receiving vehicle or OMX-CV.

| | | Vehicle | OMX-CV | p- |
|---|---|---|---|---|
| Parameters | Time point | Avg ± SD | Avg ± SD | value |
| Hgb | | 9.38 ± 1.2 | 9.32 ± 1.5 | 0.83 |
| PaO₂ | Hypoxia Bsln | 18 ± 2.3 | 21 ± 4.3 | 0.28 |
| | Hypoxia 60 min | 22.7 ± 1.7 | 21.7 ± 2.1 | 0.78 |
| Systolic | Hypoxia Bsln | 127.9 ± 24 | 119.3 ± 28.5 | 0.65 |
| SAP | Hypoxia 60 min | 105.5 ± 23.9 | 102.43 ± 29.7 | 0.77 |
| Diastolic | Hypoxia Bsln | 64.1 ± 19.7 | 60.4 ± 18.7 | 0.98 |
| SAP | Hypoxia 60 min | 42.0 ± 10.5 | 48.9 ± 19.8 | 0.55 |
| Mean SAP | Hypoxia Bsln | 81.9 ± 19.9 | 81.3 ± 17.3 | 0.83 |
| | Hypoxia 60 min | 61.3 ± 14 | 67.7 ± 19.1 | 0.60 |
| HR | Hypoxia Bsln | 158.0 ± 19.5 | 169.6 ± 44.9 | 0.50 |
| | Hypoxia 60 min | 193.4 ± 28.2 | 184.1 ± 24.3 | 0.75 |
| Systolic | Hypoxia Bsln | 35.3 ± 4.0 | 38.0 ± 9.4 | 0.57 |
| PAP | Hypoxia 60 min | 36.0 ± 3.3 | 39.5 ± 8.2 | 0.38 |
| Diastolic | Hypoxia Bsln | 15.4 ± 3.9 | 13.5 ± 3.8 | 0.46 |
| PAP | Hypoxia 60 min | 16.2 ± 2.3 | 15.7 ± 3.8 | 0.92 |
| Mean PAP | Hypoxia Bsln | 24.0 ± 3.5 | 23.7 ± 4.8 | 0.81 |
| | Hypoxia 60 min | 25.0 ± 2.3 | 25.9 ± 4.7 | 0.68 |
| LAP | Hypoxia Bsln | 3.5 ± 1.9 | 5.7 ± 1.9 | 0.07 |
| | Hypoxia 60 min | 4.4 ± 1.7 | 5.7 ± 1.6 | 0.02 |
| RAP | Hypoxia Bsln | 2.8 ± 1.2 | 4.6 ± 1.5 | 0.05 |
| | Hypoxia 60 min | 4 8 ± 2.5 | 4.9 ± 1.8 | 0.94 |
| iLPAQ | Hypoxia Bsln | 0.051 ± .006 | 0.056 ± .012 | 0.36 |
| | Hypoxia 60 min | 0.052 ± .007 | 0.055 ± .011 | 0.56 |
| iLPAVR | Hypoxia Bsln | 414.9 ± 107.1 | 332.2 ± 70.1 | 0.12 |
| | Hypoxia 60 min | 402.3 ± 62.3 | 375.3 ± 97.9 | 0.46 |

Abbreviations: Avg, average; Bsln, baseline; Hgb, hemoglobin; HR, heart rate; iLPAQ, indexed left pulmonary artery flow; iLPAVR, indexed left pulmonary artery vascular resistance; LAP, left atrial pressure; PaO₂, arterial oxygen tension; PAP, pulmonary artery pressure; RAP, right atrial pressure; SAP, systemic arterial pressure.

8.3.2 OMX-CV Administration does not Cause Systemic or Pulmonary Vasoconstriction

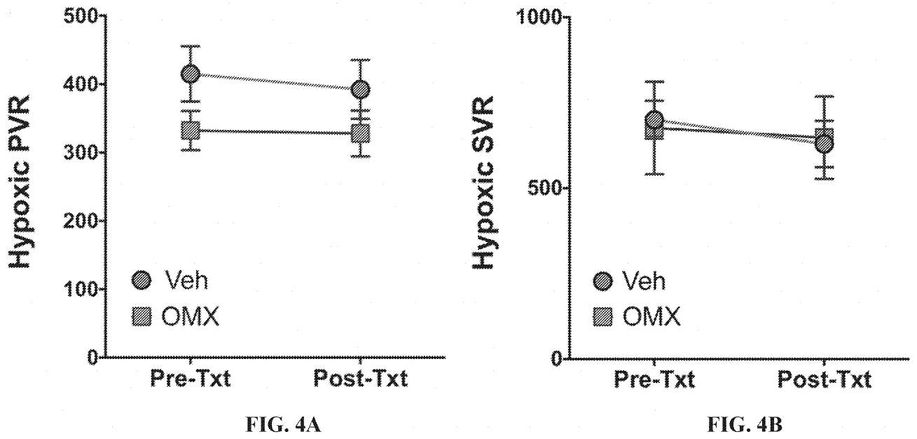

Taking into consideration the historical challenges related to NO scavenging encountered in the use of hemoglobin-based oxygen carriers (HBOCs), the physiologic impact of OMX-CV administration was evaluated on systemic and pulmonary vascular reactivity. Importantly, the total amount of OMX-CV administered relative to circulating Hb is quite low. In an average 10 kg lamb with a serum Hb concentration of 10 g/dL and a circulating blood volume of 70 mL/kg, the Hb O₂ carrying capacity is approximately 4.8 mM. In this Example, approximately 54 mL in total of OMX-CV infusion were provided, which corresponds to an infused OMX-CV O₂binding capacity of approximately 0.1 mM, or 2% binding capacity of circulating Hb. As noted in Table 2, this does not result in appreciable differences in circulating PaO₂ values but is readily available for oxygenating severely hypoxic tissues. Given the substantial physiologic changes induced by the hypoxic stimulus, the effects on SVR and PVR in the setting of systemic hypoxia prior to and immediately following drug or vehicle administration (control group, n=7 and OMX-CV group, n=6) were evaluated. As seen in FIG. 4, no significant increase was observed in either the indexed PVR (FIG. 4A) or indexed SVR (FIG. 4B) with administration of OMX-CV when compared with vehicle control under hypoxic conditions. Furthermore, there was no difference in the absolute value or percent change between the OMX-CV-treated and vehicle-treated groups. While hypoxia clearly results in a pre-constricted pulmonary vasculature, this occurs through a NO independent mechanism, and PVR would be expected to remain quite sensitive to abrupt changes in NO signaling (Blitzer M. L. et al. (1996) J. Am. Coll. Cardiol. 28:591-596; Arteel G. E. et al. (1998) Eur. J. Biochem. 253.743-750). Additionally, SVR is also increased during hypoxia, as evidenced by increased mean systemic pressure, and was similarly unaffected by OMX-CV administration (FIG. 4B), affirming a lack of direct vasoreactivity.

### 8.3.3 OMX-CV Decreases Myocardial Hypaxia.

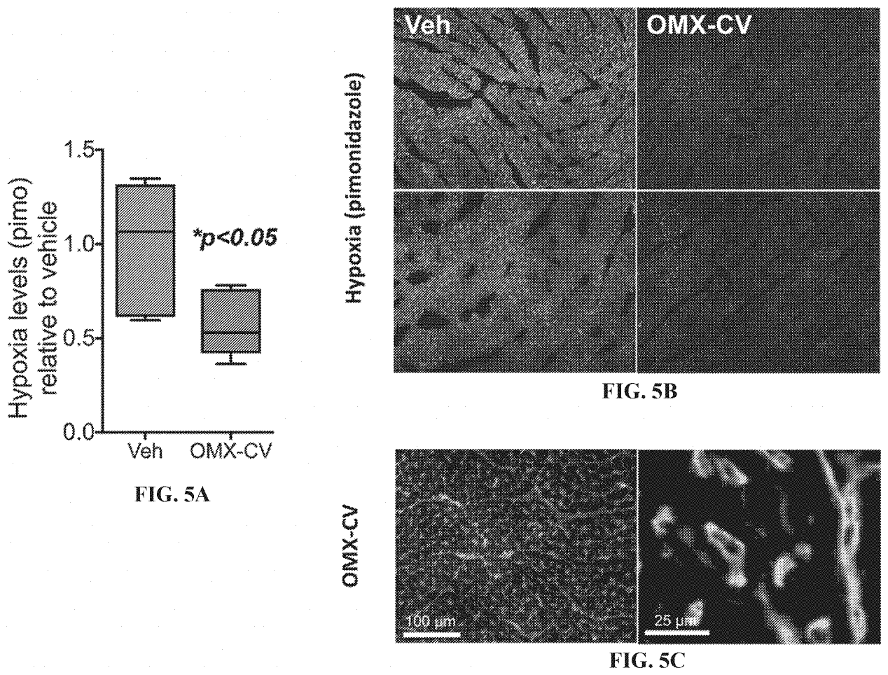

To directly assess the effect of OMX-CV on myocardial tissue oxygenation, following the final assessment of physiologic parameters, pimonidazole (Hypoxyprobe, 85 mg/kg), a well-established marker of tissue hypoxia (Suga H. et al. (1973) Circ. Res. 32:314-322), was administered intravenously to a subset of animals (n=3 per treatment group). Thirty minutes after administration of pimonidazole, the animals were humanely killed and tissues collected for processing and measurement of pimonidazole adduct levels in the ventricular myocardium. Pimonidazole freely diffuses into cells and is competitively metabolized via oxidative or reductive chemical reactions, depending on the tissue O₂ content. In severely hypoxic environments (below 10 mm Hg), reductive metabolism is favored and in its reduced state, pimonidazole forms covalent adducts with sulfhydryl groups of proteins and glutathione, leading to accumulation of pimonidazole adducts inside the cell (Suga H. et al. (1973) Circ. Res. 32:314-322). Pimonidazole adducts can be recognized using pimonidazole-targeted primary antibodies and quantified using standard ELISA and immunofluorescent (IF) methods. As seen in FIGS. 5A and 5B, the OMX-CV-treated animals exhibited a significant reduction in myocardial hypoxia compared with controls, as evidenced by lower levels of bound pimonidazole observed via IF microscopy and quantified by ELISA. To verify that the improved myocardial tissue oxygenation in the OMX-CV group was mediated by transcapillary O₂ diffusion, rather than vascular extravasation, IF microcopy was performed with antibodies directed against OMX-CV. As seen in FIG. 5C, OMX-CV was localized within the capillary vascular spaces throughout the heart and not the extracellular spaces surrounding the cardiomyocytes. Thus, at tested doses, a high-affinity O₂ delivery biotherapeutic can relieve tissue hypoxia in the heart.

### 8.3.4 OMX-CV Preserves Myocardial Contractility During Systemic Hypoxia.

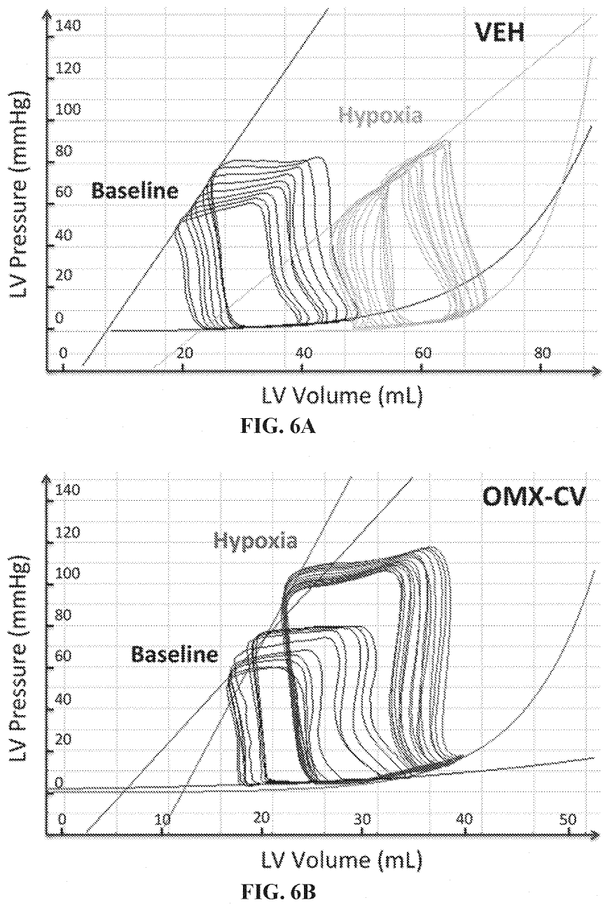

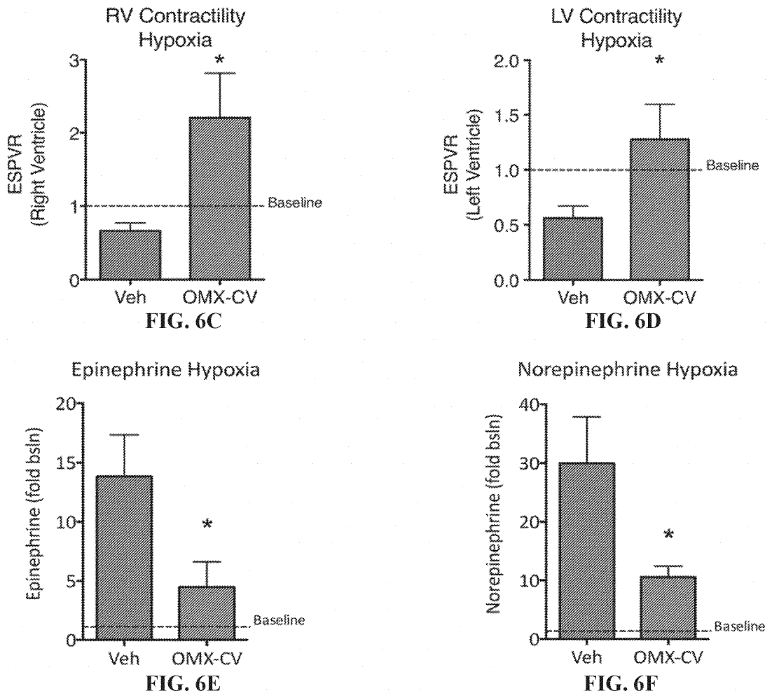

To determine whether this improvement in myocardial O₂ delivery translates into a physiologic benefit, cardiac pressure volume loop analysis was utilized to evaluate contractile function of the bilateral ventricles. As noted previously by others groups, evaluation of cardiac function in intact animal studies is often obscured by compensatory physiologic alterations to ventricular loading conditions and sympathetic tone (Walley K. R. et al. (1988) Circ. Res. 63:849-859; Moudgil R. et al. (2005) J. Appl. Physiol. (1985) 98.390-403). Here, it was observed that from the onset of hypoxia, both the OMX-CV and control groups exhibited similar elevations in cardiac output (about 15%) above the normoxic baseline, and that this was sustained throughout the study in this Example (FIG. 3). This suggests a full mobilization of compensatory mechanisms that may account for the lack of a significant difference in cardiac output between the OMX-CV and control groups at early time points Initially advanced by Suga and Sagawa in the 1970s (Suga H. et al. (1973) Circ. Res. 32:314-322), evaluation of two-dimensional ventricular pressure-volume (PV) loops with a focus on the end systolic pressure-volume relationship (ESPVR) is now the widely adopted standard used to assess the load-independent contractile state of the ventricles (Baan J. et al. (1992) Eur. Heart J. 13 Suppl. E:2-6). This method has previously been used to validate the hypoxic depression of myocardial contractile function in dogs and shown to correlate closely with myocardial O₂ deficiency and the onset of anaerobic metabolism (Walley K. R. et al. (1997) Am. J. Resptr. Crit. Care Med. 155: 222-228; Walley K. R. et al. (1988) Circ. Res. 63:849-859). In order to delineate the ESPVR, a family of loops was generated (as seen in FIGS. 6A and 6B) through transient preload suppression induced by graduated occlusion of the inferior vena cava (IVC). The slope of the tangent connecting the end systolic points of these loops gives the most precise representation of intrinsic contractility of the ventricle. As seen in FIG. 6A, which shows a representative set of loops and their ESPVR from the LV of a control animal, the decline in slope from baseline (left side of FIG. 6A, closer to x-axis) to hypoxia (right side of FIG. 6A, further away from x-axis) demonstrates a decrease in contractility. In contrast, the LV loops of an OMX-CV-treated animal (FIG. 6B) exhibit an increasing slope, indicating an improvement in contractile function. By normalizing the slope of the ESPVR at 60 minutes to the baseline for each animal (control group, n=7 and OMX-CV group, n=6), it was observed that OMX-CV-treated animals maintained an average contractility up to 2-fold above their own baseline under hypoxic conditions (FIG. 6C), while RV (FIG. 6C) and LV (FIG. 6D) contractility were both reduced in vehicle controls. These data indicate that OMX-CV treatment was able to reverse the effects of myocardial hypoxia and preserve cardiac contractility.

The role of sympathetic activation in the cardiovascular response to acute alveolar hypoxia was also explored by measuring plasma levels of the sympathetic hormones epinephrine and norepinephrine at baseline and after 60 minutes of hypoxia. Released by the adrenal medulla in response to increased stimulation of the sympathetic nervous system, these hormones exhibit potent cardiovascular effects mediated through binding of alpha- and betaadrenergic receptors in the heart and vasculature. Similar to what has been described (Downing S. E. et al. (1969) Am. J. Physiol. 217: 728-735), a significant increase in the levels of these catecholamines under hypoxic stress was noted, marking an activated sympathetic response. Interestingly, a significant difference in the levels of epinephrine and norepinephrine between the OMX-CV- and vehicle-treated animals was found (control group, n=7 and OMX-CV group, n=6), with hypoxia inducing an approximately 3-fold higher increase in both hormones in the vehicle group compared with OMX-CV (FIGS. 6E and 6F). Thus, increased adrenergic signaling was not responsible for the improved myocardial contractility of OMX-CV-treated animals compared with the control group, although the improved performance in the presence of the lower induction of catecholamines suggests a greater capacity of the OMX-CV-treated myocardium to respond to adrenergic signaling under hypoxic stress. It can therefore be concluded that while cardiac output can be maintained during severe acute alveolar hypoxia through diverse adaptive mechanisms, OMX-CV directly improves the intrinsic contractile function of the heart by virtue of its ability to increase myocardial O₂ content.

### 8.4 Conclusion and Discussion

This Example shows that OMX-CV-treated animals exhibit preserved contractility despite smaller increases in catecholamine levels (relative to vehicle-treated animals). The improved myocardial performance in the presence of lower induction of catecholamines suggests a greater capacity of the H-NOX-treated animals to respond to adrenergic signaling under hypoxic stress.

In addition, this Example provides preclinical data highlighting the therapeutic efficacy of the OMX-CV biotherapeutic in relieving hypoxic myocardial dysfunction in a large animal model. H-NOX-based variants can be suited for O₂ delivery to hypoxic tissues, such as the myocardium, because of their O₂ affinity as well as pharmacokinetic and safety profiles (LeMoan N. et al. (2017) Neuroprotective Therapy for Stroke and Ischemic Disease 641-664). The O₂ affinity of OMX-CV aligns extremely well with the unique O₂ demands and microenvironments encountered within the stressed heart. In addition, the half-life of OMX-CV enables long-term efficacy following single intravenous infusion, and its O₂ specificity minimizes the vasoactive side effects encountered with HBOCs.

The cardiovascular system responds to acute hypoxia by attempting to augment and enhance systemic O₂ delivery. Cardiac output increases with accompanying elevations in both HR and contractile state, which further escalate myocardial O₂ demand. In response to the high and variable demand for O₂ during states of acute stress, as well as the tight interrelationship between myocardial function and O₂ supply, the heart has evolved robust adaptive mechanisms to augment myocardial O₂ delivery and extraction (Duncker D. J. et al. (2015) Prog. Cardiovasc Dis. 57:409-422). For example, during exercise-induced elevations in cardiac output, Oz utilization may increase by greater than 5-fold, supported by substantial increases in coronary blood flow, capillary recruitment, and increased O₂ extraction (von Restorff W et al. (1977) Pflugers Arch. 372-181-185). Even under unstressed conditions, the heart exhibits a high O₂ extraction ratio with a correspondingly low venous saturation. When demand increases, the heart has a unique capacity to increase extraction to a greater extent than other tissues (Walley K. R. et al. (1997) Am. J. Respir. Crit. Care Med. 155: 222-228).

Global hypoxic hypoxia and anemic hypoxia induce global anaerobic metabolism at greatly differing values of mixed venous partial pressure of oxygen (PO₂) (Cain S. M. (1977) J. App. Physiol. Respir. Environ. Exerc. Physiol. 42: 228-234) These differences in tissue responses to the same level of hypoxia in the blood implied that simple diffusion forces are not the limiting factor to tissue O₂ extraction. A constant critical O₂ extraction ratio exists in dogs (Schumacker P. T. et al. (1987) J. Appl. Physiol. (1985) 62:1801-1807). Although the exact mechanisms underlying these differences are unclear, the physiologic consequence is that most tissues will start to experience O₂ deficiency despite a relatively high average O₂ saturation of the blood exiting their capillaries. In contrast to the other tissues and organs, the myocardium can achieve a substantially higher O₂ extraction ratio, only exhibiting signs of anaerobic metabolism at a critically low coronary venous saturation (Walley K. R. et al. (1997) Am. J. Respir. Crit. Care Med. 155: 222-228). This markedly hypoxic venous and end capillary blood reflects a correspondingly hypoxic tissue bed, creating a cellular microenvironment to facilitate O₂ dissociation and delivery by OMX-CV. Here it was shown that in the stressed, hypoxic lamb heart, myocardial oxygenation and contractile function can be preserved with the administration of OMX-CV, even if a small amount of OMX-CV is administered, as shown herein. This is particularly remarkable given that the total amount of OMX-CV used in this Example equates to only approximately 2% of the total O₂ carrying capacity of the circulating Hb, and this small amount of OMX-CV administered relative to total circulating Hb serves to limit any potential negative impact on total O₂ bioavailability.

Furthermore, the high O₂ affinity of OMX-CV precludes O₂ delivery under non-hypoxic conditions. This is in marked contrast to the less avid delivery profile of Hb and most HBOCs, which have been shown to contribute to pathologic hyperoxygenation of tissue and circulatory microenvironments (Winslow R. M. (2008) Biochim. Biophys. Acta 1784(10):1382-6). This excessive O₂ release has been shown to cause oxidative stress to the tissues through the production of toxic reactive oxygen species (ROS) and to induce detrimental microvascular shunting mechanisms that may inappropriately impair tissue perfusion. Delivery of excess O₂ in the setting of shock is a frequent contributor to microcirculatory shunting with significant clinical consequences (Kuiper J. W. et al. (2016) Crit. Care 20:352). While vascular indices can frequently be normalized within the macrocirculation in the setting of shock, tissue perfusion can nevertheless be compromised because of shunting at the microcirculatory level. Importantly, in adult patients with severe sepsis and traumatic hemorrhagic shock, for example, the loss of coherence between the resuscitated macrocirculation and the microcirculation is one of the most sensitive and specific hemodynamic indicators associated with increased multi-organ failure and mortality (Sakr Y. et al. (2004) Crit. Care Med. 32:1825-1831, De Backer D. et al. (2004) Am. Heart J. 147:91-99; De Backer D. et al. (2013) Crit. Care Med. 41:791-799; Tachon G. et al. (2014) Crit. Care Med. 42:1433-1441). Similarly, in critically ill children with sepsis, a persistently altered microcirculation has been associated with increased mortality (Top, 2011). OMX-CV allows a more targeted delivery of O₂ to only the most hypoxic tissue beds and may help alleviate the underappreciated but significant morbidities associated with excessive tissue oxygenation in this setting.

This Example shows that OMX-CV administration was associated with a smaller increase in circulating catecholamine levels in the setting of systemic hypoxia. While it is unclear what exactly underlies this difference in catecholamine production and release, it does suggest potential implications related to cardiac function. Hypoxia is a well-established stimulus for catecholamine secretion both in vitro and in vivo Cheung C. Y. (1989) J. Neurochem. 52:148-153; Donnelly D. F and Doyle T. P (1994)J. Physiol. 475:267-275; Kumar G. K. et al. (1998) Am. J. Physiol. 274:C1592-1600), and adrenergic responses to hypoxic stress are important for the maintenance of cardiorespiratory homeostasis (Gamboa A. et al. (2006) Clin. Auton. Res. 16:40-45; Kanstrup I. L. et al. (1999) J. Appl. Physiol. (1985) 87:2053-2058). In the perinatal period, catecholamine production by adrenomedullary chromaffin cells is directly stimulated by cellular hypoxia (Salman S. et al. (2014) J. Exp. Biol. 217:673-681; Richter S. et al. (2013) Adv Pharmacol. 68:285-317). However, as mammals age, this primary cellular response to O₂ is blunted and cholinergic innervation becomes the predominant regulatory mechanism (Kumar G. K. et al. (2015) Adv. Exp. Med. Biol. 860.195-199). The sympathetic response to hypoxia therefore matures to reflect the integrated input from peripheral and central chemoreceptors. In the juvenile lamb model of systemic hypoxia used in this Example, OMX-CV administration appears to blunt hypoxia-driven catecholamine production. It is not clear if this reflects augmented O₂ delivery to chemoreceptors or the chromaffin cells themselves, or perhaps represents some secondary mechanism related to more favorable hemodynamics associated with improved myocardial oxygenation. Importantly, in the control animals, diminished cardiac contractility is observed despite dramatically elevated levels of circulating catecholamines, while the OMX-CV-treated animals exhibit preserved contractility despite smaller increases in catecholamine levels. Epinephrine and norepinephrine are potent inotropes, vital to the regulation of cardiac contractility and hemodynamic function in response to physiologic stress. In this Example, it was shown that OMX-CV supports preservation of the cardiac response to these key regulators, which are important not only as endogenous hormones but also as exogenous agents heavily utilized for cardiovascular support in critical care medicine.

With respect to its safety profile, OMX-CV exhibits significant advantages over previously developed Hb-based O₂ carriers (HBOCs) Cabrales P. and Intaglietta M. (2013) ASAIO J. 59:337-354). As the protein responsible for storage and transport of O₂ in red blood cells (RBCs) (Lehninger A. L. et al. (2013), Lehninger principles of biochemistry. New York: W H. Freeman), Hb has been the precursor for the synthesis and formulation of HBOCs previously developed as RBC substitutes (Gould S. A. et al. (1998) J. Am. Coll. Surg. 187:113-120; Moore E. E. et al. (2009) J. Am. Coll. Surg. 208:1-13; Greenburg A. G. et al. (2004) J. Am. Coll. Surg. 198:373-383; Jahr J. S. et al. (2008) Expert Opin. Bio. Ther. 8:1425-1433). The first HBOC to be developed in this capacity consisted of partially purified “stroma-free” Hb (Gilligan D. R. et al. (1941)J Clin. Invest. 20:177-187). However, transfusion of acellular Hb led to several major side effects (Bulow L. and Alayash A. I. (2017) Antioxid Redox Signal 10; 26(14):745-747: Bunn H. F. et al. (1969) J. Exp. Med 129:909-923; Chan W. L. et al. (2000) Toxicol. Pathol. 28:635-642; Dunne J. et al. (2006) Biochem. J. 399:513-524; Zhang L. et al. (1991) J. Biol. Chem. 266.24698-24701). Extracellular tetrameric Hb readily dissociates into two pairs of dimers (Bunn, 1969; Chan, 2000), which are extremely prone to oxidation (Zhang L. et al. (1991) Biol. Chem. 266:24698-24701) and enhanced renal excretion (Bunn H. F. et al. (1969) J. Exp. Med 129:909-923; Bunn H. F. and Jandl J. H. (1969) J. Exp. Med. 129:925-934). Hb oxidation to methemoglobin (metHb) promotes unfolding of the globin chains and releases cytotoxic heme into the circulation, leading to kidney tubule damage and eventual renal failure (Bunn H. F. et al. (1969) J. Exp. Med. 129:909-923; Chan W. L. et al. (2000) Toxicol. Pathol. 28:635-642). Furthermore, metHb can no longer carry O₂ and can also contribute to the generation of harmful ROS (Bulow L. and Alayash A. I. (2017) Antioxid Redox Signal 10; 26(14):745-747; Dunne J. et al. (2006) Biochem. J. 399:513-524). Additionally, extracellular Hb can trigger vasoconstriction and systemic hypertension by various mechanisms (Winslow R. M. (2008) Biochim. Biophys. Acta 1784(10):1382-6; Kavdia M. et al. (2002) Am. J. Physiol. Heart Circ. Physio. 282.112245-2253; Gibson Q. H. and Roughton F. J., (1965) Proc. R. Soc. Lond. B. Biol. Sci. 163:197-205). Foremost amongst these is the indiscriminate scavenging of NO, an important intrinsic vasodilator that is locally produced by endothelial cells to relax vascular smooth muscle (Kavdia M. et al. (2002) Am. J. Physiol. Heart Circ. Physiol. 282:H2245-2253; Gibson Q. H. and Roughton F. J. (1957) J. Physiol. 136:507-524). Also, potentially important is the hyperoxygenation of local vasculature that can elicit inappropriate vasoconstriction within the microcirculation, compared to more tempered O₂ delivery into the vessel lumen from physiologic RBC-encapsulated Hb (Winslow R. M. (2008) Biochim. Biophys. Acta 1784(10):1382-6; Cabrales P. and Intaglietta M. (2013) ASAIO J. 59:337-354). Overall, the presence of extracellular Hb in the circulation may lead to direct tissue toxicity via heme release and ROS generation, while simultaneously impairing blood flow because of pathologic alterations in vasomotor tone. With its unique structure and O₂-binding characteristics, OMX-CV averts the potential for many of these deleterious side effects. In this Example, a lack of direct vasoreactivity in both the systemic and pulmonary vascular beds was shown, providing strong evidence for selective O₂ delivery in severely hypoxic microenvironments and lack of vasoactivity.

In summary, this Example presents preclinical data from a large animal model highlighting the therapeutic efficacy of a novel O₂ delivery biotherapeutic agent. OMX-CV, in relieving hypoxic myocardial dysfunction. OMX-CV is ideally suited for myocardial O₂ delivery because of its unique O₂-binding characteristics and safety profile. Its high O₂ affinity complements the unique O₂ demands and microenvironments encountered within the stressed heart, while its low reactivity with NO minimizes the vasoactive side effects encountered with HBOCs. Additionally, while exogenous O₂ administration can increase systemic arterial O₂ content, it can also result in microvascular shunting mechanisms that limit deep tissue oxygenation (Ince C. and Mik E. G. (2016) J. Appl. Physiol. (1985) 120:226-235; Kanoore Edul V. S. and Ince C., Dubin A. (2015) Curr. Opin. Crit. Care 21.245-252).

### 8.5. References

Kutko M. C., Calarco M. P., Flaherty M. B., Helmrich R. F., Ushay H. M., et al., 2003, “Mortality rates in pediatric septic shock with and without multiple organ system failure,” Pediatr. Crit. Care Med. 4:333-337.
Martin G. S., 2012, “Sepsis, severe sepsis and septic shock: changes in incidence, pathogens and outcomes,” Expert Rev. Anti. Infect. Ther. 10:701-706.
Heckbert S. R., Vedder N. B., Hoffman W., Winn R. K., Hudson L. D., et al., 1998, “Outcome after hemorrhagic shock in trauma patients,” J. Trauma 45:545-549.
Reynolds H. R., Hochman J. S., 2008, “Cardiogenic shock: current concepts and improving outcomes,” Circulation 117:686±697.
Taegtmeyer H., 1994, “Energy metabolism of the heart: from basic concepts to clinical applications,” Curr. Probl. Cardiol. 19:59-113.
Neely J R., Rovetto M. J., Oram J. F., 1972. “Myocardial utilization of carbohydrate and lipids,” Prog. Cardiovasc. Dis. 15:289-329.
von Restorff W., Holtz J., Bassenge E., 1977, “Exercise induced augmentation of myocardial oxygen extraction in spite of normal coronary dilatory capacity in dogs,” Pflugers Arch. 372:181-185.
Walley K. R., Collins R. M., Cooper D. J., Warriner C. B., 1997, “Myocardial anaerobic metabolism occurs at a critical coronary venous PO₂ in pigs.” Am. J. Respir. Crit. Care Med. 155:222-228.
Cain S M., 1977, “Oxygen delivery and uptake in dogs during anemic and hypoxic hypoxia,” J. Appl. Physiol. Respir. Environ. Exerc. Physiol. 42: 228-234.
Walley K. R., Becker C. J, Hogan R. A., Teplinsky K., Wood L. D., 1988, “Progressive hypoxemia limits left ventricular oxygen consumption and contractility,” Circ. Res. 63:849-859.
Karow D. S., Pan D., Tran R., Pellicena P., Presley A., et al., 2004, “Spectroscopic characterization of the soluble guanylate cyclase-like heme domains from Vibro cholerae and Thermoanaerobacter tengcongensis.” Biochemistry 43:10203-10211.
Boon E. M., Marietta M. A., 2005, “Ligand discrimination in soluble guanylate cyclase and the H-NOX family of heme sensor proteins,” Curr. Opin. Chem. Biol 9:441-446.
LeMoan N., Leung P. Y., Rost N., Winger J A., Krtolica A et al, 2017, “A New Paradigm in Protecting Ischemic Brain: Preserving the Neurovascular Unit Before Reperfusion,” Neuroprotective Therapy for Stroke and Ischemic Disease 641-664.
Chen J. Y., Scerbo M., Kramer G., 2009, “A review of blood substitutes: examining the history, clinical trial results, and ethics of hemoglobin-based oxygen carriers.” Clinics (Sao Paulo) 64:803-813.
Natanson C., Kern S. J., Lurie P., Banks S. M., Wolfe S. M., 2008, “Cell-free hemoglobin-based blood substitutes and risk of myocardial infarction and death: a meta-analysis,” JAMA 299:2304-2312.
Olson J. S., Foley E. W., Rogge C., Tsai A. L., Doyle M. P., et al., 2004, “No scavenging and the hypertensive effect of hemoglobin-based blood substitutes,” Free Radic. Biol. Med. 36:685-697.
The PyMOL Molecular Graphics System, Version 1.5 Schrödinger, LLC
Helmerhorst H. J., Schultz M. J., van der Voort P. H., de Jonge E., van Westerloo D. J., 2015, “Bench-to-bedside review: the effects of hyperoxia during critical illness,” Crit. Care 19:284.
Rudolph A. M., 2009, “Congenital diseases of the heart: clinical-physiological considerations,” Chichester, UK; Hoboken, N. J.; Wiley-Blackwell p. 538.
Kontos H. A., Mauck H. P. Jr., Richardson D. W., Patterson J L. Jr., 1965, “Mechanism of Circulatory Responses to Systemic Hypoxia in the Anesthetized Dog,” Am. J. Physiol. 209:397-403.
Downing S. E., Gardner T. H., Rocamora J. M., 1969, “Adrenergic support of cardiac function during hypoxia in the newborn lamb,” Am. J. Physiol. 217: 728-735.
Moudgil R., Michelakis E. D., Archer S. L., 2005, “Hypoxic pulmonary vasoconstriction,” J. Appl. Physiol. (1985) 98:390-403.
Aaronson P. I., Robertson T. P., Ward J. P., 2002, “Endothelium-derived mediators and hypoxic pulmonary vasoconstriction,” Respir. Physiol. Neurobiol. 132:107-120.
Blitzer M. L., Loh E., Roddy M. A., Stamler J. S., Creager M. A., 1996, “Endothelium-derived nitric oxide regulates systemic and pulmonary vascular resistance during acute hypoxia in humans,” J. Am. Coll. Cardiol. 28:591-596.
Arteel G. E., Thurman R. G., Raleigh J. A., 1998, “Reductive metabolism of the hypoxia marker pimonidazole is regulated by oxygen tension independent of the pyridine nucleotide redox state,” Eur. J. Biochem. 253:743-750.
Suga H., Sagawa K., Shoukas A. A., 1973, “Load independence of the instantaneous pressure-volume ratio of the canine left ventricle and effects of epinephrine and heart rate on the ratio,” Circ. Res. 32:314-322.
Baan J., van der Velde E. T., Steendijk P., 1992, “Ventricular pressure-volume relations in vivo,” Eur. Heart J. 13 Suppl. E:2-6.
Duncker D. J, Koller A., Merkus D., Canty J. M. Jr., 2015, “Regulation of coronary blood flow in health and ischemic heart disease,” Prog. Cardiovasc. Dis. 57:409-422.
Schumacker P. T., Long G. R., Wood L. D., 1987, “Tissue oxygen extraction during hypovolemia: role of hemoglobin P50,” J. Appl. Physiol. (1985) 62:1801-1807.
Winslow R. M., 2008, “Cell-free oxygen carriers: scientific foundations, clinical development, and new directions,” Biochim. Biophys. Acta 1784(10) 1382-6.
Kuiper J. W., Tibboel D, Ince C., 2016, “The vulnerable microcirculation in the critically ill pediatric patient,” Crit. Care 20:352.
Sakr Y., Dubois M. J., De Backer D., Creteur J., Vincent J. L., 2004, “Persistent microcirculatory alterations are associated with organ failure and death in patients with septic shock,” Crit. Care Med. 32:1825-1831.
De Backer D., Creteur J., Dubois M. J., Sakr Y., Vincent J. L., 2004, “Microvascular alterations in patients with acute severe heart failure and cardiogenic shock,” Am. Heart J. 147:91-99.
De Backer D., Donadello K., Sakr Y., Ospina-Tascon G., Salgado D., et al., 2013, “Microcirculatory alterations in patients with severe sepsis: impact of time of assessment and relationship with outcome,” Crit. Care Med. 41: 791-799.
Tachon G., Harrois A., Tanaka S., Kato H., Huet O., et al., 2014, “Microcirculatory alterations in traumatic hemorrhagic shock,” Crit. Care Med. 42:1433-1441.
Top A. P., Ince C., de Meij N., van Dijk M., Tibboel D., 2011, “Persistent low microcirculatory vessel density in nonsurvivors of sepsis in pediatric intensive care,” Crit. Care Med. 39:8-13.
Cheung C. Y., 1989, “Direct adrenal medullary catecholamine response to hypoxia in fetal sheep,” J. Neurochem. 52:148-153
Donnelly D. F., Doyle T. P., 1994, “Developmental changes in hypoxia-induced catecholamine release from rat carotid body, in vitro,” J. Physiol. 475:267-275.
Kumar G. K, Overholt J. L., Bright G. R., Hui K. Y., Lu H., et al., 1998, ‘Release of dopamine and norepinephrine by hypoxia from PC-12 cells,” Am. J Physiol. 274:C1592-1600.
Gamboa A., Gamboa J. L., Holmes C., Sharabi Y., Leon-Velarde F., et al., 2006, “Plasma catecholamines and blood volume in native Andeans during hypoxia and normoxia,” Clin. Auton. Res. 16:40-45.
Kanstrup I. L., Poulsen T. D., Hansen J. M., Andersen L. J., Bestle M. H., et al., 1999, “Blood pressure and plasma catecholamines in acute and prolonged hypoxia: effects of local hypothermia,” J. Appl. Physiol. (1985) 87:2053-2058.
Salman S., Buttigieg J., Nurse C. A., 2014, “Ontogeny of O₂ and CO2/H+ chemosensitivity in adrenal chromaffin cells: role of innervation,” J. Exp. Biol. 217:673-681.
Richter S., Qin N., Pacak K., Eisenhofer G., 2013, “Role of hypoxia and HITF2alpha in development of the sympathoadrenal cell lineage and chromaffin cell tumors with distinct catecholamine phenotypic features,” Adv. Pharmacol. 68:285-317.
Kumar G. K., Peng Y. J., Nanduri J., Prabhakar N. R., 2015, “Carotid Body Chemoreflex Mediates Intermittent Hypoxia-Induced Oxidative Stress in the Adrenal Medulla,” Adv. Exp. Med. Biol. 860:195-199.
Cabrales P., Intaglietta M., 2013, “Blood substitutes: evolution from noncarrying to oxygen- and gas-carrying fluids,” ASAIO J. 59:337-354.
Lehninger A L., Nelson D L., Cox M. M., 2013, Lehninger principles of biochemistry. New York: W. H Freeman.
Gould S. A., Moore E. E., Hoyt D. B., Burch J M., Haenel J. B., et al., 1998, “The first randomized trial of human polymerized hemoglobin as a blood substitute in acute trauma and emergent surgery,” J. Am. Coll. Surg. 187:113-120; discussion 120-112.
Moore E. E., Moore F. A., Fabian T. C., Bernard A. C., Fulda G. J., et al., 2009, “Human polymerized hemoglobin for the treatment of hemorrhagic shock when blood is unavailable: the USA multicenter trial,” J. Am. Coll. Surg. 208:1-13.
Greenburg A. G., Kim H. W., Hemolink Study Group, 2004, “Use of an oxygen therapeutic as an adjunct to intraoperative autologous donation to reduce transfusion requirements in patients undergoing coronary artery bypass graft surgery,” J. Am. Coll. Surg. 198:373-383; discussion 384-385
Jahr J. S., Moallempour M., Lim J. C., 2008, “HBOC-201, hemoglobin glutamer-250 (bovine), Hemopure (Biopure Corporation),” Expert Opin. Biol. Ther. 8:1425-1433.
Gilligan D. R., Altschule M. D., Katersky E. M., 1941, “Studies of Hemoglobinemia and Hemoglobinuria Produced in Man by Intravenous Injection of Hemoglobin Solutions,” J. Clin. Invest. 20:177-187.
Bulow L., Alayash A. I., 2017, “Redox Chemistry of Hemoglobin-Associated Disorders,” Antioxid. Redox. Signal 10; 26(14):745-747.
Bunn H. F., Esham W. T., Bull R. W., 1969, “The renal handling of hemoglobin I. Glomerular filtration,” J. Exp. Med. 129:909-923.
Chan W. L., Tang N. L., Yim C. C., Lai F. M., Tam M. S., 2000, “New features of renal lesion induced by stroma free hemoglobin,” Toxicol. Pathol. 28:635-642.
Dunne J., Caron A., Menu P., Alayash A. I., Buehler P. W., et al., 2006, “Ascorbate removes key precursors to oxidative damage by cell-free haemoglobin in vitro and in vivo,” Biochem. J. 399:513-524.
Zhang L., Levy A., Rifkind J. M., 1991, “Autoxidation of hemoglobin enhanced by dissociation into dimers,” J. Biol. Chem. 266:24698-24701.
Bunn H. F., Jandl J. H., 1969, “The renal handling of hemoglobin. II. Catabolism,” J. Exp. Med. 129:925-934.
Kavdia M., Tsoukias N. M., Popel A S., 2002, “Model of nitric oxide diffusion in an arteriole: impact of hemoglobin-based blood substitutes,” Am. J. Physiol. Heart Circ. Physiol. 282:H2245-2253.
Gibson Q. H., Roughton F. J., 1965, “Further studies on the kinetics and equilibria of the reaction of nitric oxide with haemoproteins,” Proc. R. Soc. Lond. B. Biol. Sci. 163:197-205.
Gibson Q. H., Roughton F. J., 1957, “The kinetics and equilibria of the reactions of nitric oxide with sheep haemoglobin,” J Physiol. 136:507-524.
Ince C., Mik E. G., 2016, “Microcirculatory and mitochondrial hypoxia in sepsis, shock, and resuscitation,” J. Appl. Physiol. (1985) 120:226-235.
Kanoore Edul V. S., Ince C., Dubin A., 2015, “What is microcirculatory shock?” Curr. Opin. Crit. Care 21:245-252.
Porterfield J. E., Kottam A. T. G., Raghavan K., Escobedo D. Jenkins J. T., et al., 2009, “Dynamic correction for parallel conductance, GP, and gain factor, a, in invasive murine left ventricular volume measurements,” J. Appi Physiol. 107:1693-1703.
Kleiter M. M., Thrall D. E., Malarkey D. E., Ji X., Lee D. Y., et al., 2006, “A comparison of oral and intravenous pimonidazole in canine tumors using intravenous CCI-103F as a control hypoxia marker,” Int. J. Radiat. Oncol. Biol. Phys. 64:592-602.
Krishnamoorthy V., Hiller D. B., Ripper R., Lin B., et al., 2012, “Epinephrine induces rapid deterioration in pulmonary oxygen exchange in intact, anesthetized rats.” Anesthesiology 117(4):745-754.

### 9. SEQUENCES

Nucleic acid sequences are presented 5′ to 3′
Amino acid sequences are presented N-terminus to C-terminus.

Thermoanaerobacter tengcongensis HNOX-wild type
Nucleic acid
(SEQ ID NO: 1)
ATGAAGGGGACAATCGTCGGGACATGGATAAAGACCCTGAGGGACCTTTACGGGAATGA
TGTGGTTGATGAATCTTTAAAAAGTGTGGGTTGGGAACCAGATAGGGTAATTACACCTC
TGGAGGATATTGATGACGATGAGGTTAGGAGAATTTTTGCTAAGGTGAGTGAAAAAACT
GGTAAAAATGTCAACGAAATATGGAGAGAGGTAGGAAGGCAGAACATAAAAACTTTCAG
CGAATGGTTTCCCTCCTATTTTGCAGGGAGAAGGCTAGTGAATTTTTTAATGATGATGG
ATGAGGTACACCTACAGCTTACCAAGATGATAAAAGGAGCCACTCCTCCAAGGCTTATT
GCAAAGCCTGTTGCAAAAGATGCCATTGAAATGGAGTACGTTTCTAAAAGAAAGATGTA
CGATTACTTTTTAGGGCTTATAGAGGGTAGTTCTAAATTTTTCAAGGAAGAAATTTCAG
TGGAAGAGGTCGAAAGAGGCGAAAAAGATGGCTTTTCAAGGCTAAAAGTCAGGATAAAA
TTTAAAAACCCCGTTTTTGAGTGA
Amino acid
(SEQ ID NO: 2)
MKGTIVGTWIKTLRDLYGNDVVDESLKSVGWEPDRVITPLEDIDDDEVRRIFAKVSEKT
GKNVNEIWREVGRQNIKTFSEWFPSYFAGRRLVNFLMMMDEVHLQLTKMIKGATPPRLI
AKPVAKDAIEMEYVSKRKMYDYFLGLIEGSSKFFKEEISVEEVERGEKDGFSRLKVRIK
FKNPVFW
Foldon domain
Nucleic acid
(SEQ ID NO: 3)
ggttatattcctgaagctccaagagatgggcaagcttacgttcgtaaagatggcgaatg
ggtattactttctaccttttta
Amino acid
(SEQ ID NO: 4)
GYIPEAPRDGQAYVRKDGEWVLLSTFPL
Thermoanaerobacter tengcongensis H-NOX-L144F
Nucleic acid
(SEQ ID NO: 5)
ATGAAGGGGACAATCGTCGGGACATGGATAAAGACCCTGAGGGACCTTTACGGGAATGA
TGTGGTTGATGAATCTTTAAAAAGTGTGGGTTGGGAACCAGATAGGGTAATTACACCTC
TGGAGGATATTGATGACGATGAGGTTAGGAGAATTTTTGCTAAGGTGAGTGAAAAAACT
GGTAAAAATGTCAACGAAATATGGAGAGAGGTAGGAAGGCAGAACATAAAAACTTTCAG
CGAATGGTTTCCCTCCTATTTTGCAGGGAGAAGGCTAGTGAATTTTTTAATGATGATGG
ATGAGGTACACCTACAGCTTACCAAGATGATAAAAGGAGCCACTCCTCCAAGGCTTATT
GCAAAGCCTGTTGCAAAAGATGCCATTGAAATGGAGTACGTTTCTAAAAGAAAGATGTA
CGATTACTTTTTAGGGTTTATAGAGGGTAGTTCTAAATTTTTCAAGGAAGAAATTTCAG
TGGAAGAGGTCGAAAGAGGCGAAAAAGATGGCTTTTCAAGGCTAAAAGTCAGGATAAAA
TTTAAAAACCCCGTTTTTGAGTGA
Amino acid
(SEQ ID NO: 6)
MKGTIVGTWIKTLRDLYGNDVVDESLKSVGWEPDRVITPLEDIDDDEVRRIFAKVSEKT
GKNVNEIWREVGRQNIKTFSEWFPSYFAGRRLVNFLMMMDEVHLQLTKMIKGATPPRLI
AKPVAKDAIEMEYVSKRKMYDYFLGFIEGSSKFFKEEISVEEVERGEKDGFSRLKVRIK
FKNPVFE
Thermoanaerobacter tengcongensis H-LOX-L144F-foldon
Nucleic acid
(SEQ ID NO: 7)
atgaaggggacaatcgtcgggacatggataaagaccctgagggacctttacgggaatga
tgtggttgatgaatctttaaaaagtgtgggttgggaaccagatagggtaattacacctc
tggaggatattgatgacgatgaggttaggagaatttttgctaaggtgagtgaaaaaact
ggtaaaaatgtcaacgaaatatggagagaggtaggaaggcagaacataaaaactttcag
cgaatggtttccctcctattttgcagggagaaggctagtgaattttttaatgatgatgg
atgaggtacacctacagcttaccaagatgataaaaggagccactcctccaaggcttatt
gcaaagcctgttgcaaaagatgccattgaaatggagtacgtttctaaaagaaagatgta
cgattactttttagggtttatagagggtagttctaaatttttcaaggaagaaatttcag
tggaagaggtcgaaagaggcgaaaaagatggcttttcaaggctaaaagtcaggataaaa
tttaaaaaccccgtttttgagtataagaaaaatctcgagggcagcggcggttatattcc
tgaagctccaagagatgggcaggcttacgttcgtaaagatggcgaatgggtattacttt
ctacctttttatga
Amino acid
(SEQ ID NO: 8)
MKGTIVGTWIKTLRDLYGNDVVDESLKSVGWEPDRVITPLEDIDDDEVRRIFAKVSEKTGKN
VNEIWREVGRQNIKTFSEWFPSYFAGRRLVNFLMMMDEVHLQLTKMIKGATPPRLIAKPVAK
DAIEMEYVSKRKMYDYFLGFIEGSSKFFKEEISVEEVERGEKDGFSRLKVRIKFKNPVFEYK
KNLEGSGGYIPEAPRDGQAYVRKDGEWVLLSTFPL

### INCORPORATION BY REFERENCE

Various references such as patents, patent applications, and publications are cited herein, the disclosures of which are hereby incorporated by reference herein in their entireties.

1. A method for treating a cardiovascular disorder or pulmonary disorder in a subject in need thereof, said method comprising administering to the subject (a) an H-NOX protein; and (b) a catecholamine.

2. The method of claim 1, wherein the cardiovascular disorder or pulmonary disorder is associated with hypoxia.

3. The method of claim 1, which is for treating a cardiovascular disorder.

4. (canceled)

5. The method of claim 1, which is for treating a pulmonary disorder.

6-8. (canceled)

9. The method of claim 1, wherein the H-NOX protein is a polymeric H-NOX protein comprising (i) an H-NOX domain of T. tengcongensis H-NOX with an L144F amino acid substitution, and (ii) a polymerization domain.

10. The method of claim 1, wherein administering the H-NOX protein comprises administering a mixture comprising (i) an H-NOX protein covalently bound to polyethylene glycol (PEG), and (ii) an H-NOX protein not bound to PEG.

11. The method of claim 10, wherein the mixture has a weight ratio of the H-NOX protein covalently bound to PEG to the H-NOX protein not bound to PEG of about 9:1, about 8:2, about 7:3, about 6:4, about 1:1, about 4:6, about 3:7, about 2:8, or about 1:9.

12. (canceled)

13. The method of claim 10, wherein the H-NOX protein covalently bound to PEG and/or the H-NOX protein not bound to PEG is a polymeric H-NOX protein comprising (i) an H-NOX domain of T. tengcongensis H-NOX with an L144F amino acid substitution, and (ii) a polymerization domain.

14. The method of claim 9, wherein the polymeric H-NOX protein comprises monomers, each monomer being a fusion protein comprising the H-NOX domain fused via a peptide linker to the polymerization domain.

15. The method of claim 9, wherein the polymeric H-NOX protein is a trimeric H-NOX protein comprising three monomers, wherein each of the monomers comprises the H-NOX domain and a trimerization domain.

16. The method of claim 15, wherein the trimerization domain is a foldon domain of bacteriophage T4 fibritin.

17. (canceled)

18. The method of claim 14, wherein each monomer has the amino acid sequence of SEQ ID NO:8.

19. The method of claim 15, wherein the trimeric H-NOX comprises three PEG molecules per monomer.

20-21. (canceled)

22. The method of claim 1, wherein administering the H-NOX protein comprises administering OMX-CV.

23. The method of claim 1, wherein the H-NOX protein is administered before, concurrently with, or after the administration of the catecholamine.

24-25. (canceled)

26. A pharmaceutical composition comprising (i) an H-NOX protein or a mixture of H-NOX protein, and (ii) a catecholamine.

27. An infusion bag comprising the composition of claim 26.

28. A method for treating a cardiovascular disorder or pulmonary disorder in a subject in need thereof, said method comprising administering to the subject an H-NOX protein.

29-50. (canceled)

51. The method of claim 1 wherein the H-NOX protein or mixture of H-NOX proteins is present in a therapeutically effective amount within a pharmaceutical composition.

52-71. (canceled)

72. The method of claim 1, wherein the catecholamine is epinephrine, norepinephrine, dopamine, dobutamine, or atropine.

73-77. (canceled)
